(19) United States
(12) Patent Application Publication
(10) Pub. No.: US 2025/0019714 A1
(43) Pub. Date: Jan. 16, 2025

(54) METHODS FOR REDUCING NITROUS OXIDE PRODUCTION

(71) Applicant: AGRESEARCH LIMITED, Lincoln (NZ)

(72) Inventors: Nicholas John ROBERTS, Feilding (NZ); Gregory BRYAN, Ashurst (NZ); Luke James COONEY, Palmerston North (NZ); Michele Lee REID, Palmerston North (NZ); Somrutai WINICHAYAKUL, Palmerston North (NZ); Zachariah BEECHEY-GRADWELL, Ashurst (NZ); Willora Mudiyanselage Saman Deepal BOWATTE, Palmerston North (NZ); Paul Charles Dummere NEWTON, Palmerston North (NZ); Coby Jean HOOGENDOORN, Awapuni (NZ)

(21) Appl. No.: 18/724,423

(22) PCT Filed: Dec. 19, 2022
(86) PCT No.: PCT/IB2022/062440

§ 371 Date: Jun. 26, 2024

(30) Foreign Application Priority Data
Dec. 21, 2021 (AU) 2021904161

(51) Int. Cl. C12N 15/82; C07K 14/415

(52) U.S. Cl. CPC C12N 15/8247; C07K 14/415; C12N 15/8259

(57) ABSTRACT

The invention provides methods for producing plants that reduce nitrous oxide (N₂O) production from the soil in which they are grown. The invention involves expressing modified oleosins with artificially introduced cysteine residues in the plants. The plants optionally also express a triacylglycerol (TAG) synthesising enzyme. The invention also provides methods for reducing nitrous oxide (N₂O) production from the soil in which the plants are grown. The invention also includes methods for the production of seed of the plants, and packages and use of such seed.

### CROSS REFERENCE TO RELATED APPLICATIONS

The contents of Australian provisional patent application number 2021904161, filed 21 Dec. 2021, is incorporated herein by reference in its entirety.

### TECHNICAL FIELD

The invention is in the field of agronomy and relates to reducing nitrous oxide production from soil in which plants are grown.

### BACKGROUND

Nitrous oxide (N₂O) has a global warming potential almost 300 times higher than CO₂ (Signor and Cerri 2013). In addition, N₂O also depletes stratospheric ozone and N₂O emission is currently the single most important ozone depleting emission. As such, it is expected that N₂O will remain the largest ozone depleting emission throughout the 21^{st} century (Velthof and Rietra 2018).

Of the mineral elements that are essential for plants, Nitrogen (N) is required in the greatest quantity and plays a critical role in attaining the high yields required of modern agriculture (Andrews et al., 2013). However, it is estimated that around half of the global anthropogenic N₂O emissions arise from agricultural endeavours, particularly N fertilization and animal excreta (Uchida and von Rein 2018).

There are several types of Enhanced Efficiency Fertilizers (EEFs) which help reduce N₂O emissions from the nitrification and denitrification of fertilizers. Some EFEs contain nitrification or urease inhibitors (stabilized fertilizers), while others slowly release N components (slow-release fertilizers) or release N at more predictable rates (controlled-release fertilizers). In addition, the use of ammonium-based fertilizers with a nitrification inhibitor can further reduce N₂O emission. The most well-known nitrification inhibitors, dicyandiamide (DCD), and 3, 4-dimethypyrazole phosphate (DMPP) can reduce N₂O emission by approximately 30 to 50%, with the highest effects seen in grassland. Although several compounds may have inhibitory effects on denitrification, none are available commercially. Moreover, for EEFs and nitrification inhibitors to have the maximum effect they all require careful control over application rate, timing and placement.

As an example of N sources that contribute to N₂O emissions Ju et al (2015) highlighted that in the last 50 years the world's rice yield has continuously increased, partly because of the increase in fertilizer nutrient input, especially N fertilizer (Cassman et al., 2003; Peng et al., 2009). However, the use of N fertilizer is generally inefficient, and the apparent Recovery Efficiency of N fertilizer (REN, the percentage of fertilizer N recovered in aboveground plant biomass at the end of the cropping season) is only 33%, on average (Raun and Johnson, 1999; Garnett et al., 2009). The remaining N is lost as either surface runoff, leached nitrate in groundwater, volatilization to the atmosphere or by microbial denitrification (Vitousek et al., 1997). High N input, togehther with low Nitorgen Use Efficiency (NUE) not only increases production costs, but also results in severe environmental pollution (Peng et al., 2009; Guo et al., 2010; X. P. Chen et al., 2014).

Once N enters the soil the formation of N₂O occurs mainly through nitrification and denitrification processes, which are influenced by soil moisture, temperature, oxygen concentration, amount of available organic carbon (C) and N as well as the soil C/N ratio. Higher soil C/N ratios are generally associated with reduced N₂O emissions (Cayuela et al 2014; de Klein et al 2020). In general, plant roots control and influence Soil Organic Carbon (SOC) dynamics by providing the majority of organic C to the soil primarily in the forms of root litter and rhizodeposition (Dijkstra et al., 2020). This C input results in SOC gain; particularly when plant roots promote the stabilization of SOC (Rasse et al., 2005).

To this end, there are numerous examples where breeding has focussed on increasing root biomass and overall plant NUE (York et al 2013; Chen et al., 2014; Ju et al., 2015; Pang et al., 2015).

It is an object of the invention to provide methods for producing plants with capacity to reduce N₂O emissions and/or that overcome one or more of the limitations of methods of the prior art and/or at least provide the public with a useful choice.

### SUMMARY OF THE INVENTION

The applicants have surprisingly shown that plants genetically modified to express modified oleosins with artificially introduced cysteine residues, and optionally also to express a triacylglycerol (TAG) synthesising enzyme, when grown in soil, can significantly reduce nitrous oxide (N₂O) production from the soil.

The invention therefore provides methods for producing plants that reduce nitrous oxide (N₂O) production from the soil in which they are grown. The invention involves expressing modified oleosins with artificially introduced cysteine residues in the plants. The plants optionally also express a triacylglycerol (TAG) synthesising enzyme. The invention also provides methods for reducing nitrous oxide (N₂O) production from the soil in which the plants are grown.

Producing Plants that Reduce N₂O Production

In the first aspect the invention provides a method for producing a plant that reduces N₂O production from the soil in which it is grown, the method comprising the step of genetically modifying the plant to express a modified oleosin including at least one artificially introduced cysteine.

In a further embodiment the plant is also genetically modified to express at least one triacylglycerol (TAG) synthesising enzyme.

### Method Includes Step of Measuring N₂O Production

In one embodiment the method comprise the additional step of assessing the capability of the plant produced to reduce N₂O production from the soil in which it is grown, and selecting the plant based on this assessment.

### Reducing N₂O Production

In a further aspect the invention provides a method for reducing N₂O production in soil the method comprising growing a plant produced by a method of the invention in soil.

In a further aspect the invention provides a method for reducing N₂O production in soil the method comprising growing a plant comprising a modified oleosin including at least one artificially introduced cysteine produced in soil.

### Reduction is Relative

In one embodiment the reduction in N₂O production from the soil in which the plant is grown is relative to N₂O production from the same soil in the absence of the plant.

In a further embodiment the reduction in N₂O production from the soil in which the plant is grown is relative to N₂O production in the same soil from which a control plant is grown.

### Level of Reduction in N₂O Production

In one embodiment N₂O production is reduced by at least 1%, preferably at least 2%, more preferably at least 3%, more preferably at least 4%, more preferably at least 5%, more preferably at least 10%, more preferably at least 15%, more preferably at least 20%, more preferably at least 25%, more preferably at least 30%, more preferably at least 35%, more preferably at least 40%, more preferably at least 45%, more preferably at least 50%, more preferably at least 55%, more preferably at least 60%, more preferably at least 65%, more preferably at least 70%, more preferably at least 75%, more preferably at least 80%, more preferably at least 85%, more preferably at least 90%, more preferably at least 95%.

In one embodiment the reduction in N₂O production is assessed in soil in which multiple plants are grown relative to that in the same soil in which the same number of control plants are grown.

### Period of Reduction in N₂O Production

In one embodiment N₂O production is reduced over a period of at least 1 day, preferably at least 2 days, more preferably at least 3 days, more preferably at least 4 days, more preferably at least 5 days, more preferably at least 6 days, more preferably at least 1 week, more preferably at least 2 weeks, more preferably at least 3 weeks, more preferably at least 4 weeks, more preferably at least 1 month, more preferably at least 2 months, more preferably at least 3 months, more preferably at least 4 months, more preferably at least 5 months, more preferably at least 6 months, more preferably at least 7 months, more preferably at least 8 months, more preferably at least 9 months, more preferably at least 10 months, more preferably at least 11 months, more preferably at least 1 year of the period in which the plant is grown in the soil.

### Nitrogen (N) in the Soil

In one embodiment the soil has a Nitrogen (N) load of at least 10 kg N/ha, preferably at least 20 kg N/ha, more preferably at least 30 kg N/ha, more preferably at least 40 kg N/ha, more preferably at least 50 kg N/ha, more preferably at least 100 kg N/ha, more preferably at least 200 kg N/ha, more preferably at least 250 kg N/ha, more preferably at least 300 kg N/ha, more preferably at least 350 kg N/ha, more preferably at least 400 kg N/ha, more preferably at least 450 kg N/ha, more preferably at least 500 kg N/ha, more preferably at least 550 kg N/ha, more preferably at least 600 kg N/ha, more preferably at least 650 kg N/ha, more preferably at least 700 kg N/ha, more preferably at least 750 kg N/ha, more preferably at least 800 kg N/ha, more preferably at least 850 kg N/ha, more preferably at least 900 kg N/ha, more preferably at least 1000 kg N/ha.

In one embodiment the soil has a Nitrogen (N) load in the range 200-1000 kg N/ha, preferably in the range 300-900 kg N/ha, more preferably in the range 400-800 kg N/ha.

In one embodiment the soil has a Nitrogen (N) load in the range 100-700 kg N/ha, preferably in the range 200-600 kg N/ha, more preferably in the range 300-500 kg N/ha, more preferably in the range 350-450 kg N/ha.

In a further embodiment the soil has a Nitrogen (N) load of about 400 kg N/ha.

In a further embodiment the soil has a Nitrogen (N) load of 400 kg N/ha.

In one embodiment the soil has a Nitrogen (N) load in the range 600-1000 kg N/ha, preferably in the range 700-900 kg N/ha, more preferably in the range 750-850 kg N/ha.

In a further embodiment the soil has a Nitrogen (N) load of about 800 kg N/ha.

In a further embodiment the soil has a Nitrogen (N) load of 800 kg N/ha.

In one embodiment the Nitrogen (N) loads above, are in addition to Nitrogen (N) present in the soil prior to loading.

The additional N may be a component of a fertiliser or soil additive, or may be in the urine or faecal matter of animals either grazing on the plants or supplied as effluent. The additional N may also be from artificial urine as described herein.

### Genetic Modification of Plants to Express a Modified Oleosin Including at Least One Artificially Introduced Cysteine

In one embodiment the method includes the step of modifying an endogenous oleosin-encoding polynucleotide in the plant to produce a polynucleotide encoding the modified oleosin. Methods for modifying endogenous polynucleotides are well known to those skilled in the art, and are described further herein.

In one embodiment the method includes the step of introducing into the plant, a polynucleotide encoding a modified oleosin including at least one artificially introduced cysteine.

In one embodiment the method includes the step of transforming the plant with a polynucleotide encoding the modified oleosin including at least one artificially introduced cysteine.

### Genetic Modification of Plants to Express at Least One Triacylglycerol (TAG) Synthesising Enzyme.

In one embodiment the method includes the step of modifying an endogenous TAG sythesising gene in the plant to bring about increased expression of the TAG synthesising enzyme. For example, modification of regulatory sequences in the gene can be modified to increase expression of the TAG synthesising enzyme. Methods for modifying endogenous polynucleotides are well known to those skilled in the art, and are described further herein.

In one embodiment the method includes the step of introducing into the plant, a polynucleotide encoding the TAG synthesising enzyme.

In one embodiment the method includes the step of transforming the plant with a polynucleotide encoding the TAG synthesising enzyme.

### Polynucleotide is Part of a Genetic Construct

In one embodiment the polynucleotide encoding the modified oleosin, or TAG synthesising enzyme, is transformed as part of a genetic construct. Preferably the genetic construct is an expression construct. Preferably the expression construct includes the polynucleotide operably linked to a promoter. In a further embodiment the polynucleotide is operably linked to a terminator sequence.

### Promoters for Plants

In one embodiment the promoter operably linked to the polynucleotide is capable of driving expression of the polynucleotide in a photosynthetic tissue of a plant. In one embodiment the promoter is a photosynthetic cell preferred promoter. In a further embodiment the promoter is a photosynthetic cell specific promoter. In a further embodiment the promoter is capable of driving expression of the polynucleotide in a green tissue of a plant. In one embodiment the promoter is a green tissue preferred promoter. In a further embodiment the promoter is a green tissue specific promoter. In a further embodiment the promoter is capable of driving expression of the polynucleotide in a vegetative photosynthetic tissue of a plant. In a further embodiment the promoter is capable of driving expression of the polynucleotide in a leaf of a plant.

It will be understood by those skilled in the art that the polynucleotide encoding the modified oleosin and the nucleic acid sequence encoding a triacylglycerol (TAG) synthesising enzyme can be placed on the same construct or on separate constructs to be transformed into the plant. Expression of each can be driven by the same or different promoters, which may be included in the construct to be transformed. It will also be understood by those skilled in the art that alternatively the polynucleotide and nucleic acid can be transformed into the plant without a promoter, but expression of either or both of the polynucleotide and nucleic acid could be driven by a promoter or promoters endogenous to the plant transformed.

### Modified Oleosin

In one embodiment, the modified oleosin includes at least two cysteines, at least one of which is artificially introduced. In a further embodiment, the modified oleosin includes at least two to at least thirteen (i.e., 2, 3, 4, 5, 6, 7, 8, 9, 10, 11, 12, 13, 14 or more) artificially introduced cysteines. In one embodiment the cysteines are artificially introduced into the N-terminal hydrophilic region of the oleosin, or into the C-terminal hydrophilic region of the oleosin. In a further embodiment the modified oleosin includes at least one cysteine in the N-terminal hydrophilic region, and at least one cysteine in the C-terminal hydrophilic region. In a further embodiment the cysteines are distributed substantially evenly over the N-terminal and C-terminal hydrophilic regions of the oleosin. In a further embodiment the cysteines are distributed evenly over the N-terminal and C-terminal hydrophilic regions of the oleosin. In a further embodiment the introduced cysteine replaces an existing charged amino acid.

Preferably the modified oleosin includes at least one artificially introduced cysteine, wherein the cysteine is introduced into at least one of:
a) in the N-terminal hydrophilic region of the oleosin, and
b) in the C-terminal hydrophilic region of the oleosin.

### Source of Oleosins, TAG Synthesising Enzymes, and Plants

The modified oleosins may be modified naturally occurring oleosins. The TAG synthesising enzymes may be naturally occurring, or modified from naturally occurring TAG synthesising enzymes. The plants from which the un-modified oleosin and TAG synthesising enzyme sequences are derived may be from any plant species that contains oleosins and TAG synthesising enzymes and polynucleotide sequences encoding oleosins and TAG synthesising enzymes.

The plant cells, in which the modified oleosins oleosins and TAG synthesising enzymes are expressed, may be from any plant species. The plants, in which the modified oleosins oleosins and TAG synthesising enzymes are expressed, may be from any plant species.

In one embodiment the plant cell or plant, is derived from a gymnosperm plant species. In a further embodiment the plant cell or plant, is derived from an angiosperm plant species. In a further embodiment the plant cell or plant, is derived from a from dicotyledonous plant species. In a further embodiment the plant cell or plant, is derived from a monocotyledonous plant species.

Preferred plant species are those that produce tubers (modified stems) such as but not limited to Solanum species. Other preferred plant species are those that produce bulbs (below ground storage leaves) such as but not limited to Lilaceae, Amaryllis, Hippeastrum, Narcissus, Iridaceae, and Oxalis species. Other preferred plant species are those that produce corms (swollen underground stems) such as but not limited to Musa, Elocharis, Gladiolus and Colocasia species. Other preferred plant species are those that produce rhizomes (underground storage stem) such as but not limited to Asparagus, Zingiber and Bambuseae species. Other preferred are those that produce substantial endosperm in their seeds, such as but not limited to maize and sorghum.

Preferred plants incude those from the following genera: Brassica, Solanum, Raphanus, Allium, Foeniculum, Lilaceae, Amaryllis, Hippeastrum, Narcissus, Iridaceae, Oxalis, Musa, Eleocharis, Gladiolus, Colocasia, Asparagus, Zingiber, and Bambuseae.

A preferred Brassica species is Brassica rapa var. rapa (turnip)

Preferred Solanum species are those which produce tubers. A preferred Solanum species is Solanum tuberosum (potato)

Preferred Raphanus species include Raphanus raphanistrum, Raphanus caudatu, and Raphanus sativus. A preferred Raphanus species is Raphanus sativus (radish)

Preferred Allium species include: Allium cepa (onion, shallot), Allium fistulosum (bunching onion), Allium schoenoprasum (chives), Allium tuberosum (Chinese chives), Allium ampeloprasum (leek, kurrat, great-headed garlic, pearl onion), Allium sativum (garlic) and Allium chinense (rakkyo). A preferred Allium species is Allium cepa (onion)

Preferred Musa species include: Musa acuminata and Musa balbisiana. A preferred Musa species is Musa acuminata (banana, plantains)

A preferred Zingiber species is Zingiber officinale (ginger)

A preferred Oxalis species is Oxalis tuberosa (yam)

A preferred Colocasia species is Colocasia esculenta (taro).

Another preferred genera is Zea. A preferred Zea species is Zea mays.

Another preferred genera is Sorghum. A preferred Sorghum species is Sorghum bicolor.

Other preferred plants are forage plant species from a group comprising but not limited to the following genera: Zea, Lolium, Hordium, Miscanthus, Saccharum, Festuca, Dactylis, Bromus, Thinopyrum, Trifolium, Medicago, Pheleum, Phalaris, Holcus, Glycine, Lotus, Plantago and Cichorium.

Other preferred plants are leguminous plants. The leguminous plant or part thereof may encompass any plant in the plant family Leguminosae or Fabaceae. For example, the plants may be selected from forage legumes including, alfalfa, clover; leucaena; grain legumes including, beans, lentils, lupins, peas, peanuts, soy bean; bloom legumes including lupin, pharmaceutical or industrial legumes; and fallow or green manure legume species.

A particularly preferred genus is Trifolium. Preferred Trifolium species include Trifolium repens; Trifolium arvense; Trifolium affine; and Trifolium occidentale. A particularly preferred Trifolium species is Trifolium repens.

Another preferred genus is Medicago. Preferred Medicago species include Medicago sativa and Medicago truncatula. A particularly preferred Medicago species is Medicago sativa, commonly known as alfalfa.

Another preferred genus is Glycine. Preferred Glycine species include Glycine max and Glycine wightii (also known as Neonotonia wightii). A particularly preferred Glycine species is Glycine max, commonly known as soy bean. A particularly preferred Glycine species is Glycine wightii, commonly known as perennial soybean.

Another preferred genus is Vigna. A particularly preferred Vigna species is Vigna unguiculata commonly known as cowpea.

Another preferred genus is Mucana. Preferred Mucana species include Mucana pruniens. A particularly preferred Mucana species is Mucana pruniens commonly known as velvetbean.

Another preferred genus is Arachis. A particularly preferred Arachis species is Arachis glabrata commonly known as perennial peanut.

Another preferred genus is Pisum. A preferred Pisum species is Pisum sativum commonly known as pea.

Another preferred genus is Lotus. Preferred Lotus species include Lotus corniculatus, Lotus pedunculatus, Lotus glabar, Lotus tenuis and Lotus uliginosus. A preferred Lotus species is Lotus corniculatus commonly known as Birdsfoot Trefoil. Another preferred Lotus species is Lotus glabar commonly known as Narrow-leaf Birdsfoot Trefoil. Another preferred Lotus species is Lotus pedunculatus commonly known as Big trefoil. Another preferred Lotus species is Lotus tenuis commonly known as Slender trefoil.

Another preferred genus is Brassica. A preferred Brassica species is Brassica oleracea, commonly known as forage kale and cabbage.

Other preferred species are oil seed crops including but not limited to the following genera: Brassica, Carthumus, Helianthus, Zea and Sesamum.

A preferred oil seed genera is Brassica. A preferred oil seed species is Brassica napus.

A preferred oil seed genera is Brassica. A preferred oil seed species is Brassica oleraceae.

A preferred oil seed genera is Carthamus. A preferred oil seed species is Carthamus tinctorius.

A preferred oil seed genera is Helianthus. A preferred oil seed species is Helianthus annuus.

A preferred oil seed genera is Zea. A preferred oil seed species is Zea mays.

A preferred oil seed genera is Sesamum. A preferred oil seed species is Sesamum indicum.

A preferred silage genera is Zea. A preferred silage species is Zea mays.

A preferred grain producing genera is Hordeum. A preferred grain producing species is Hordeum vulgare.

A preferred grazing genera is Lolium. A preferred grazing species is Lolium perenne.

A preferred grazing genera is Lolium. A preferred grazing species is Lolium arundinaceum.

A preferred grazing genera is Trifolium. A preferred grazing species is Trifolium repens.

A preferred grazing genera is Hordeum. A preferred grazing species is Hordeum vulgare.

Preferred plants also include forage, or animal feedstock plants. Such plants include but are not limited to the following genera: Miscanthus, Saccharum, Panicum.

A preferred biofuel genera is Miscanthus. A preferred biofuel species is Miscanthus giganteus.

A preferred biofuel genera is Arundo. A preferred biofuel species is Arundo donax.

A preferred biofuel genera is Saccharum. A preferred biofuel species is Saccharum officinarum.

A preferred biofuel genera is Panicum. A preferred biofuel species is Panicum virgatum.

In one embodiment the plant is a C3 plant.

In one embodiment the plant is selected from: rice, soybean, wheat, rye, oats, millet, barley, potato, canola, sunflower and safflower.

Preferred plants include those from the following genera: Oryza, Glycine, Hordeum, Secale, Avena, Pennisetum, Setaria, Panicum, Eleusine, Solanum, Brassica, Helianthus and Carthamus.

Preferred Oryza species include Oryza sativa and Oryza minuta.

Preferred Glycine species include Glycine max and Glycine wightii (also known as Neonotonia wightii). A particularly preferred Glycine species is Glycine max, commonly known as soybean. A particularly preferred Glycine species is Glycine wightii, commonly known as perennial soybean.

A preferred Hordeum species is Hordeum vulgare.

Preferred Triticum species include Triticum aestivum, Triticum durum and Triticum monococcum.

A preferred Secale species is Secale cereal.

A preferred Avena species is Avena sativa.

Preferred millet species include Pennisetum glaucum, Setaria italica, Panicum miliaceum and Eleusine coracana.

Preferred Solanum species include Solanum habrochaites, Solanum lycopersicum, Solanum nigrum, and Solanum tuberosum.

Preferred Brassica species include Brassica napus, Brassica campestris and Brassica Rapa.

Preferred Helianthus species include Helianthus annuus and Helianthus argophyllus.

A preferred Carthamus species is Carthamus tinctorius

In one embodiment the plant is a C4 plant.

Preferred C4 plants include those selected from the genera: Sorghum, Zea, Saccharum (sugarcane), Miscanthus and Arundo.

Preferred Sorghum species include Sorghum bicolor and Sorghum propinquum

A preferred Zea species is Zea mays (maize)

A preferred Saccharum species is Saccharum officinarum.

A preferred Arundo is Arundo donax.

Suitable control plants include non-transformed or wild-type versions of plant of the same variety and/or species as the transformed plant used in the method of the invention. Suitable control plants also include plants of the same variety and or species as the transformed plant that are transformed with a control construct. Suitable control plants also include plants that have not been transformed with a polynucleotide encoding a modified oleosin including at least one artificially introduced cysteine. Suitable control plants also include plants that do not express a modified oleosin including at least one artificially introduced cysteine.

### Seed Production, Seed, and Seed Packages

In a one embodiment the method of invention includes the step of production of seed from the plant, or plants produced.

In a further embodiment the seed is to be, or is promoted, marketed or labelled, as any one of:
a) reducing green house gas (GHG) production in soil in which the seeds, and/or resulting plants, are grown,
b) reducing N₂O production in soil in which the seeds, and/or resulting plants, are grown,
c) having environmental, social, and governance (ESG) benefits as a result of a) or b).

In a further embodiment the invention provides a plant or seed produced by the method of the invention that is promoted or marketed as any one of:
a) reducing green house gas (GHG) production in soil in which the plant or seed is grown,
b) reducing N₂O production in soil in which the plant or seed is grown,
c) having environmental, social, and governance (ESG) benefits as a result of a) or b).

In a further embodiment the invention provides a package containing seed produced by the method of the invention promoted, marketed or labelled, as any one of:
a) reducing green house gas (GHG) production in soil in which the seeds, and/or resulting plants, are grown,
b) reducing N₂O production in soil in which the seeds, and/or resulting plants, are grown,
c) having environmental, social, and governance (ESG) benefits as a result of a) or b).

In a further embodiment the invention provides a plant or seed produced by the method of the invention for use in reducing N₂O production from the soil in which it is grown.

In a further embodiment the invention provides a plant or seed produced by the method of of the invention when used to reduce N₂O production from the soil in which it is grown.

### DETAILED DESCRIPTION OF THE INVENTION

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

### Methods for Measuring Nitrous Oxide

Methods for measuring nitrous oxide production from soil, including soil in which plants are grown, are well-known to those skilled in the art (see for example: de Klein et al., 2003; Saggar et al., 2007; van Der Weerden et al., 20011).

### TAG Biosynthesis, Oil Bodies and Oleosins

On a weight for weight basis lipids have approximately double the energy content of either proteins or carbohydrates. The bulk of the world's lipids are produced by plants and the densest form of lipid is as a triacylglycerol (TAG). Dicotyledonous plants can accumulate up to approximately 60% of their seed weight as TAG which is subsequently used as an energy source for germination.

The TAG produced in developing seeds is typically contained within discreet structures called oil bodies (OBs) which are highly stable and remain as discrete tightly packed organelles without coalescing even when the cells desiccate or undergo freezing conditions (Siloto et al., 2006; Shimada et al., 2008). OBs consist of a TAG core surrounded by a phospholipid monolayer embedded with proteinaceous emulsifiers. The latter make up 0.5-3.5% of the OB; of this, 80-90% is oleosin with the remainder predominantly consisting of the calcium binding (caloleosin) and sterol binding (steroleosin) proteins (Lin and Tzen, 2004). The emulsification properties of oleosins derives from their three functional domains which consist of an amphipathic N-terminal arm, a highly conserved central hydrophobic core (˜72 residues) and a C-terminal amphipathic arm. Similarly, both caloleosin and steroleosin possess hydrophilic N and C-terminal arms and their own conserved hydrophobic core.

### Oil Bodies

OBs generally range from 0.5-2.5 μm in diameter and consist of a TAG core surrounded by a phospholipid monolayer embedded with proteinaceous emulsifiers-predominantly oleosins (Tzen, et al 1997). OBs consist of only 0.5-3.5% protein; of this 80-90% is oleosin with the remainder predominantly consisting of the calcium binding (caleosin) and sterol binding (steroleosin) proteins (Lin and Tzen, 2004). The ratio of oleosin to TAG within the plant cell influences the size and number of oil bodies within the cell (Sarmiento et al., 1997; Siloto et al., 2006).

While OBs are naturally produced predominantly in the seeds and pollen of many plants they are also found in some other organs (e.g., specific tubers).

### Oleosins

Oleosins are comparatively small (15 to 24 kDa) proteins which allow the OBs to become tightly packed discrete organelles without coalescing as the cells desiccate or undergo freezing conditions (Leprince et al., 1998; Siloto et al., 2006; Slack et al., 1980; Shimada et al.2008).

Oleosins have three functional domains consisting of an amphipathic N-terminal arm, a highly conserved central hydrophobic core (˜72 residues) and a C-terminal amphipathic arm. The accepted topological model is one in which the N- and C-terminal amphipathic arms are located on the outside of the OBs and the central hydrophobic core is located inside the OB (Huang, 1992; Loer and Herman, 1993; Murphy 1993). The negatively charged residues of the N- and C-terminal amphipathic arms are exposed to the aqueous exterior whereas the positively charged residues are exposed to the OB interior and face the negatively charged lipids. Thus, the amphipathic arms with their outward facing negative charge are responsible for maintaining the OBs as individual entities via steric hinderance and electrostatic repulsion both in vivo and in isolated preparation (Tzen et al, 1992). The N-terminal amphipathic arm is highly variable and as such no specific secondary structure can describe all examples. In comparison the C-terminal arm contains a a-helical domain of 30-40 residues (Tzen et al, 2003). The central core is highly conserved and thought to be the longest hydrophobic region known to occur in nature; at the centre is a conserved 12 residue proline knot motif which includes three spaced proline residues (for reviews see Frandsen et al, 2001; Tzen et al, 2003). The secondary, tertiary and quaternary structure of the central domain is still unclear. Modelling, Fourier Transformation-Infra Red (FT-IR) and Circular Dichromism (CD) evidence exists for a number of different arrangements (for review see Roberts et al., 2008).

The properties of the major oleosins is relatively conserved between plants and is characterised by the following:
15-25 kDa protein corresponding to approximately 140-230 amino acid residues.
The protein sequence can be divided almost equally along its length into 4 parts which correspond to a N-terminal hydrophilic region, two centre hydrophobic regions (joined by a proline knot or knob) and a C-terminal hydrophilic region.
The topology of oleosin is attributed to its physical properties which includes a folded hydrophobic core flanked by hydrophilic domains. This arrangement confers an amphipathic nature to oleosin resulting in the hydrophobic domain being embedded in the phospholipid monolayer (Tzen et al., 1992) while the flanking hydrophilic domains are exposed to the aqueous environment of the cytoplasm.
Typically oleosins do not contain cysteines

Preferred oleosins for use in the invention are those which contain a central domain of approximately 70 non-polar amino acid residues (including a proline knot) uninterrupted by any charged residues, flanked by two hydrophilic arms.

Examples of oleosin sequences suitable to be modified for use in the invention, by the addition of at least one artificially introduced cysteine, are shown in Table 1 below. The sequences (both polynucleotide and polypeptide are provided in the Sequence Listing)

TABLE 1

| | | Protein accession | SEQ | | SEQ |
|---|---|---|---|---|---|
| Sequences | Species | no. | ID NO: | cDNA accession no. | ID NO: |
| Oleosin | S. indicum | AAG23840 | 1 | AF302907 | 13 |
| Oleosin | S. indicum | AAB58402 | 2 | U97700 | 14 |
| Oleosin | A. thaliana | CAA44225 | 3 | X62353 | 15 |
| Oleosin | A. thaliana | AAZ23930 | 4 | BT023738 | 16 |
| Oleosin | H. annuus | CAA44224.1 | 5 | X62352.1 | 17 |
| Oleosin | B. napus | CAA57545.1 | 6 | X82020.1 | 18 |
| Oleosin | Z. mays | NP_001147032.1 | 7 | NM_001153560.1 | 19 |
| Oleosin | O. sativa | AAL40177.1 | 8 | AAL40177.1 | 20 |
| Oleosin | B. oleracea | AAD24547.1 | 9 | AF117126.1 | 21 |
| Oleosin | C. arabica | AAY14574.1 | 10 | AY928084.1 | 22 |
| Oleosin | B. oleraceae | CAA65272.1 | 11 | X96409 | 23 |
| Oleosin | S. indicum | AAD42942 | 12 | AF091840 | 24 |
| Oleosin | G. max | NP_001358853 | 51 | NM_001371924 | 52 |
| Oleosin | G. max | NP_001347234 | 53 | NM_001360305 | 54 |
| Oleosin | G. max | NM_001236098 | 55 | NM_001249169 | 56 |
| Oleosin | G. max | NP_001237482 | 57 | NM_001250553 | 58 |
| Oleosin | Z. mays | NP_001105338 | 59 | NM_001111868 | 60 |

Oleosin are well known to those skilled in the art. Further sequences from many different species can be readily identified by methods well-known to those skilled in the art. For example, further sequences can be easily identified by an NCBI Entrez Cross-Database Search (available at http://www.ncbi.nlm.nih.gov/sites/gquery) using oleosin as a search term.

### Plant Lipids Biosynthesis

All plant cells produce fatty acids from actetyl-CoA by a common pathway localized in plastids. Although a portion of the newly synthesized acyl chains is then used for lipid biosynthesis within the plastid (the prokaryotic pathway), a major portion is exported into the cytosol for glycerolipid assembly at the endoplasmic reticulum (ER) or other sites (the eukaryotic pathway). In addition, some of the extraplastidial glycerolipids return to the plastid, which results in considerable intermixing between the plastid and ER lipid pools (Ohlrogge and Jaworski 1997).

The simplest description of the plastidial pathway of fatty acid biosynthesis consists of two enzyme systems: acetyl-CoA carboxylase (ACCase) and fatty acid synthase (FAS). ACCase catalyzes the formation of malonyl-CoA from acetyl-CoA, and FAS transfers the malonyl moiety to acyl carrier protein (ACP) and catalyzes the extension of the growing acyl chain with malonyl-ACP.

The initial fatty acid synthesis reaction is catalyzed by 3-ketoacyl-ACP III (KAS III) which results in the condensation of acetyl-CoA and malonyl-ACP. Subsequent condensations are catalyzed by KAS I and KAS II. Before a subsequent cycle of fatty acid synthesis begins, the 3-ketoacyl-ACP intermediate is reduced to the saturated acyl-ACP in the remaining FAS reactions, catalyzed sequentially by the 3-ketoacyl-ACP reductase, 3 hydroxyacyl-ACP dehydrase, and the enoyl-ACP reductase.

The final products of FAS are usually 16:0 and 18:0-ACP, and the final fatty acid composition of a plant cell is in large part determined by activities of several enzymes that use these acyl-ACPs at the termination phase of fatty acid synthesis. Stearoyl-ACP desatruase modifies the final product of FAS by insertion of a cis double bond at the 9 position of the C18: 0-ACP. Reactions of fatty acid synthesis are terminated by hydrolysis or transfer of the acyl chain from the ACP. Hydrolysis is catalyzed by acyl-ACP thioesterases, of which there are two main types: one thioesterase relatively specific for 18:1-ACP and a second more specific for saturated acyl-ACPs. Fatty acids that have been released from ACPs by thioesterases leave the plastid and enter into the eukaryotic lipid pathway, where they are primarily esterified to glycerolipids on the ER. Acyl transferases in the plastid, in contrast to thioesterases, terminate fatty acid synthesis by transesterifying acyl moieties from ACP to glycerol, and they are an essential part of the prokaryotic lipid pathway leading to plastid glycerolipid assembly.

### Triacylglycerol Biosynthesis

The only committed step in TAG biosynthesis is the last one, i.e. the addition of a third fatty acid to an existing diacylglycerol, thus generating TAG. In plants this step is predominantly (but not exclusively) performed by one of five (predominantly ER localised) TAG synthesising enzymes including: acyl CoA: diacylglycerol acyltransferase (DGAT1); an unrelated acyl CoA: diacylglycerol acyl transferase (DGAT2); a soluble DGAT (DGAT3) which has less than 10% identity with DGAT1 or DGAT2 (Saha et al., 2006); phosphatidylcholine-sterol O-acyltransferase (PDAT); and a wax synthase (WSD1, Li et al., 2008). The DGAT1 and DGAT2 proteins are encoded by two distinct gene families, with DGAT1 containing approximately 500 amino acids and 10 predicted transmembrane domains and DGAT2 has only 320 amino acids and two transmembrane domains (Shockey et al., 2006).

The term “triacylglycerol synthesising enzyme” or “TAG synthesising enzyme” as used herein means an enzyme capable of catalysing the addition of a third fatty acid to an existing diacylglycerol, thus generating TAG. Preferred TAG synthesising enzymes include but are not limited to: acyl CoA: diacylglycerol acyltransferase1 (DGAT1); diacylglycerol acyl transferase2 (DGAT2); phosphatidylcholine-sterol O-acyltransferase (PDAT) and cytosolic soluble form of DGAT (soluble DGAT or DGAT3).

Examples of these TAG synthesising enzymes, suitable for use in the methods and compositions of the invention, from members of several plant species are provided in Table 2 below. The sequences (both polynucleotide and polypeptide are provided in the Sequence Listing)

TABLE 2

| TAG | | | | | |
|---|---|---|---|---|---|
| synthesising | | Protein | SEQ | cDNA accession | SEQ |
| enzyme | Species | accession no. | ID NO: | no. | ID NO: |
| DGAT1 | A. thaliana | NP_179535 | 25 | NM_127503 | 33 |
| DGAT1 | T. majus | AAM03340 | 26 | AY084052 | 34 |
| DGAT1 | Z. mays | ABV91586 | 27 | EU039830 | 35 |
| DGAT2 | A. thaliana | NP_566952 | 28 | NM_115011 | 36 |
| DGAT2 | B. napus | AC090187 | 29 | FJ858270 | 37 |
| DGAT3 | A. hypogaea | AAX62735 | 30 | AY875644 | 38 |
| (soluble | | | | | |
| DGAT) | | | | | |
| PDAT | A. thaliana | NP_196868 | 31 | NM_121367 | 39 |
| PDAT | R. communis | XP_002521350 | 32 | XM_002521304 | 40 |
| DGAT1 | G. max | NP_001237684 | 61 | NM_001250755 | 62 |

The inventions also contemplates use of modified TAG synthesizing enzymes, that are modified (for example in their sequence by substitutions, insertions or additions and the like) to alter their specificity and or activity.

### Modified Oleosins Engineered to Include Artificially Introduced Cysteines

The modified oleosins for use in the methods of the invention are modified to contain at least one artificially introduced cysteine residue. Preferably the engineered oleosins contain at least two cysteines.

Various methods well-known to those skilled in the art may be used in production of the modified oleosins with artificially introduced cysteines.

Such methods include site directed mutagenesis (U.S. Pat. No. 6,448,048) in which the polynucleotide encoding an oleosin is modified to introduce a cysteine into the encoded oleosin protein.

Alternatively, the polynucleotide encoding the modified oleosins, may be synthesised in its entirety.

Further methodology for producing modified oleosins and for use in the methods of the invention are described in WO/2011/053169, U.S. Pat. No. 8,987,551, and WO/2013/022353, and are provided in the Examples section of the present application.

The introduced cysteine may be an additional amino acid (i.e. an insertion) or may replace an existing amino acid (i.e. a replacement). Preferably the introduced cysteine replaces an existing amino acid. In a preferred embodiment the replaced amino acid is a charged residue. Preferably the charged residue is predicted to be in the hydrophilic domains and therefore likely to be located on the surface of the oil body.

The hydrophilic, and hydrophobic regions/arms of the oleosin can be easily identified by those skilled in the art using standard methodology (for example: Kyte and Doolitle (1982).

The modified oleosins for use in the methods of the invention are preferably range in molecular weight from 5 to 50 kDa, more preferably, 10 to 40 kDa, more preferably 15 to 25 kDa.

The modified oleosins for use in the methods of the invention are preferably in the size range 100 to 300 amino acids, more preferably 110 to 260 amino acids, more preferably 120 to 250 amino acids, more preferably 130 to 240 amino acids, more preferably 140 to 230 amino acids.

Preferably the modified oleosins comprise an N-terminal hydrophilic region, two centre hydrophobic regions (joined by a proline knot or knob) and a C-terminal hydrophilic region.

Preferably the modified oleosins can be divided almost equally their length into four parts which correspond to the N-terminal hydrophilic region (or arm), the two centre hydrophobic regions (joined by a proline knot or knob) and a C-terminal hydrophilic region (or arm).

Preferably the topology of modified oleosin is attributed to its physical properties which include a folded hydrophobic core flanked by hydrophilic domains.

Preferably the modified oleosins can be formed into oil bodies when combined with triacylglycerol (TAG) and phospholipid.

Preferably topology confers an amphipathic nature to modified oleosin resulting in the hydrophobic domain being embedded in the phospholipid monolayer of the oil body while the flanking hydrophilic domains are exposed to the aqueous environment outside the oil body, such as in the cytoplasm.

Preferably the modified oleosin includes at least one artificially introduced cysteine, wherein the cysteine is introduced into at least one of:
a) in the N-terminal hydrophilic region of the oleosin, and
b) in the C-terminal hydrophilic region of the oleosin.

In one embodiment the modified oleosin for use in the method of the invention, comprises a sequence with at least 70% identity to the hydrophobic domain of any of the oleosin protein sequences referred to in Table 1 above.

In one embodiment the modified oleosin for use in the method of the invention, comprises a sequence with at least 70% identity to the hydrophobic domain of any of the protein sequences of SEQ ID NO: 1-12.

In one embodiment the modified oleosin for use in the method of the invention, comprises a sequence with at least 70% identity to any of the oleosin protein sequences referred to in Table 1 above.

In one embodiment the modified oleosin for use in the method of the invention, comprises a sequence with at least 70% identity to any of the protein sequences of SEQ ID NO: 1-12.

In further embodiment the modified oleosin is essentially the same as any of the oleosins referred to in Table 1 above, apart from the additional artificially introduced cysteine or cysteines.

In a further embodiment the modified oleosin of the invention or used in the method of the invention, comprises a sequence with at least 70% identity to the hydrophobic domain of the oleosin sequence of SEQ ID NO: 12.

In a further embodiment the modified oleosin of the invention or used in the method of the invention, comprises a sequence with at least 70% identity to the oleosin sequence of SEQ ID NO: 12.

In further embodiment the modified oleosin has the same amino acid sequence as that of SEQ ID NO: 12, apart from the additional artificially introduced cysteine or cysteines.

In a further embodiment the modified oleosin of the invention or used in the method of the invention, comprises a sequence with at least 70% identity to the hydrophobic domain of the sequence of SEQ ID NO: 49.

In a further embodiment the modified oleosin of the invention or used in the method of the invention, comprises a sequence with at least 70% identity to the sequence of SEQ ID NO: 49.

In further embodiment the modified oleosin is has the amino acid sequence of SEQ ID NO: 49.

### Tissue/Organ Specific and Preferred Promoters

A tissue/organ preferred promoter is a promoter that drives expression of an operably linked polynucleotide in a particular tissue/organ at a higher level than in other tissues/organs. A tissue specific promoter is a promoter that drives expression of an operably linked polynucleotide specifically in a particular tissue/organ. Even with tissue/organ specific promoters, there is usually a small amount of expression in at least one other tissue. A tissue specific promoter is by definition also a tissue preffered promoter.

### Vegetative Tissues

Vegetative tissue include, shoots, leaves, roots, stems. A preferred vegetative tissue is a leaf.

### Vegetative Tissue Specific Promoters

An example of a vegetative specific promoter is found in U.S. Pat. Nos. 6,229,067; and 7,629,454; and 7,153,953; and 6,228,643.

### Pollen Specific Promoters

An example of a pollen specific promoter is found in U.S. Pat. Nos. 7,141,424; and 5,545,546; and 5,412,085; and 5,086,169; and 7,667,097.

### Seed Specific Promoters

An example of a seed specific promoter is found in U.S. Pat. Nos. 6,342,657; and 7,081,565; and 7,405,345; and 7,642,346; and 7,371,928.

### Fruit Specific Promoters

An example of a fruit specific promoter is found in U.S. Pat. Nos. 5,536,653; and 6,127,179; and 5,608,150; and 4,943,674.

### Non-Photosynthetic Tissue Preferred Promoters

Non-photosynthetic tissue preferred promoters include those preferentially expressed in non-photosynthetic tissues/organs of the plant.

Non-photosynthetic tissue preferred promoters may also include light repressed promoters.

### Light Repressed Promoters

An example of a light repressed promoter is found in U.S. Pat. No. 5,639,952 and in U.S. Pat. No. 5,656,496.

### Root Specific Promoters

An example of a root specific promoter is found in U.S. Pat. No. 5,837,848; and US 2004/0067506 and US 2001/0047525.

### Tuber Specific Promoters

An example of a tuber specific promoter is found in U.S. Pat. No. 6,184,443.

### Bulb Specific Promoters

An example of a bulb specific promoter is found in Smeets et al., (1997) Plant Physiol. 113:765-771.

### Rhizome Preferred Promoters

An example of a rhizome preferred promoter is found Seong Jang et al., (2006) Plant Physiol. 142:1148-1159.

### Endosperm Specific Promoters

An example of an endosperm specific promoter is found in U.S. Pat. No. 7,745,697.

### Corm Promoters

An example of a promoter capable of driving expression in a corm is found in Schenk et al., (2001) Plant Molecular Biology, 47:399-412.

### Photosythetic Tissue Preferred Promoters

Photosythetic tissue preferred promoters include those that are preferrentially expressed in photosynthetic tissues of the plants. Photosynthetic tissues of the plant include leaves, stems, shoots and above ground parts of the plant. Photosythetic tissue preferred promoters include light regulated promoters.

### Light Regulated Promoters

Numerous light regulated promoters are known to those skilled in the art and include for example chlorophyll a/b (Cab) binding protein promoters and Rubisco Small Subunit (SSU) promoters. An example of a light regulated promoter is found in U.S. Pat. No. 5,750,385. Light regulated in this context means light inducible or light induced.

### Relative Terms

The relative terms, such as increased and reduced as used herein with respect to plants, are relative to a control plant. Suitable control plants include non-transformed or wild-type versions of plant of the same variety and/or species as the transformed plant used in the method of the invention. Suitable control plants also include plants of the same variety and/or species as the transformed plant that are transformed with a control construct. Suitable control constructs include emptry vector constructs, known to those skilled in the art. Suitable control plants also include plants that have not been transformed with a polynucleotide encoding a modified oleosin including at least one artificially introduced cysteine. Suitable control plants also include plants that do not express a modified oleosin including at least one artificially introduced cysteine.

### Polynucleotides and Fragments

The term “polynucleotide(s),” as used herein, means a single or double-stranded deoxyribonucleotide or ribonucleotide polymer of any length but preferably at least 15 nucleotides, and include as non-limiting examples, coding and non-coding sequences of a gene, sense and antisense sequences complements, exons, introns, genomic DNA, cDNA, pre-mRNA, mRNA, rRNA, siRNA, miRNA, tRNA, ribozymes, recombinant polypeptides, isolated and purified naturally occurring DNA or RNA sequences, synthetic RNA and DNA sequences, nucleic acid probes, primers and fragments.

A “fragment” of a polynucleotide sequence provided herein is a subsequence of contiguous nucleotides that is capable of specific hybridization to a target of interest, e.g., a sequence that is at least 15 nucleotides in length. The fragments of the invention comprise 15 nucleotides, preferably at least 16 nucleotides, more preferably at least 17 nucleotides, more preferably at least 18 nucleotides, more preferably at least 19 nucleotides, more preferably at least 20 nucleotides, more preferably at least 21 nucleotides, more preferably at least 22 nucleotides, more preferably at least 23 nucleotides, more preferably at least 24 nucleotides, more preferably at least 25 nucleotides, more preferably at least 26 nucleotides, more preferably at least 27 nucleotides, more preferably at least 28 nucleotides, more preferably at least 29 nucleotides, more preferably at least 30 nucleotides, more preferably at least 31 nucleotides, more preferably at least 32 nucleotides, more preferably at least 33 nucleotides, more preferably at least 34 nucleotides, more preferably at least 35 nucleotides, more preferably at least 36 nucleotides, more preferably at least 37 nucleotides, more preferably at least 38 nucleotides, more preferably at least 39 nucleotides, more preferably at least 40 nucleotides, more preferably at least 41 nucleotides, more preferably at least 42 nucleotides, more preferably at least 43 nucleotides, more preferably at least 44 nucleotides, more preferably at least 45 nucleotides, more preferably at least 46 nucleotides, more preferably at least 47 nucleotides, more preferably at least 48 nucleotides, more preferably at least 49 nucleotides, more preferably at least 50 nucleotides, more preferably at least 51 nucleotides, more preferably at least 52 nucleotides, more preferably at least 53 nucleotides, more preferably at least 54 nucleotides, more preferably at least 55 nucleotides, more preferably at least 56 nucleotides, more preferably at least 57 nucleotides, more preferably at least 58 nucleotides, more preferably at least 59 nucleotides, more preferably at least 60 nucleotides, more preferably at least 61 nucleotides, more preferably at least 62 nucleotides, more preferably at least 63 nucleotides, more preferably at least 64 nucleotides, more preferably at least 65 nucleotides, more preferably at least 66 nucleotides, more preferably at least 67 nucleotides, more preferably at least 68 nucleotides, more preferably at least 69 nucleotides, more preferably at least 70 nucleotides, more preferably at least 71 nucleotides, more preferably at least 72 nucleotides, more preferably at least 73 nucleotides, more preferably at least 74 nucleotides, more preferably at least 75 nucleotides, more preferably at least 76 nucleotides, more preferably at least 77 nucleotides, more preferably at least 78 nucleotides, more preferably at least 79 nucleotides, more preferably at least 80 nucleotides, more preferably at least 81 nucleotides, more preferably at least 82 nucleotides, more preferably at least 83 nucleotides, more preferably at least 84 nucleotides, more preferably at least 85 nucleotides, more preferably at least 86 nucleotides, more preferably at least 87 nucleotides, more preferably at least 88 nucleotides, more preferably at least 89 nucleotides, more preferably at least 90 nucleotides, more preferably at least 91 nucleotides, more preferably at least 92 nucleotides, more preferably at least 93 nucleotides, more preferably at least 94 nucleotides, more preferably at least 95 nucleotides, more preferably at least 96 nucleotides, more preferably at least 97 nucleotides, more preferably at least 98 nucleotides, more preferably at least 99 nucleotides, more preferably at least 100 nucleotides, more preferably at least 150 nucleotides, more preferably at least 200 nucleotides, more preferably at least 250 nucleotides, more preferably at least 300 nucleotides, more preferably at least 350 nucleotides, more preferably at least 400 nucleotides, more preferably at least 450 nucleotides and most preferably at least 500 nucleotides of contiguous nucleotides of a polynucleotide disclosed. A fragment of a polynucleotide sequence can be used in antisense, RNA interference (RNAi), gene silencing, triple helix or ribozyme technology, or as a primer, a probe, included in a microarray, or used in polynucleotide-based selection methods of the invention.

The term “primer” refers to a short polynucleotide, usually having a free 3′OH group, that is hybridized to a template and used for priming polymerization of a polynucleotide complementary to the target.

The term “probe” refers to a short polynucleotide that is used to detect a polynucleotide sequence that is complementary to the probe, in a hybridization-based assay. The probe may consist of a “fragment” of a polynucleotide as defined herein.

### Polypeptides and Fragments

The term “polypeptide”, as used herein, encompasses amino acid chains of any length but preferably at least 5 amino acids, including full-length proteins, in which amino acid residues are linked by covalent peptide bonds. Polypeptides of the present invention, or used in the methods of the invention, may be purified natural products, or may be produced partially or wholly using recombinant or synthetic techniques. The term may refer to a polypeptide, an aggregate of a polypeptide such as a dimer or other multimer, a fusion polypeptide, a polypeptide fragment, a polypeptide variant, or derivative thereof.

A “fragment” of a polypeptide is a subsequence of the polypeptide that performs a function that is required for the biological activity and/or provides three dimensional structure of the polypeptide. The term may refer to a polypeptide, an aggregate of a polypeptide such as a dimer or other multimer, a fusion polypeptide, a polypeptide fragment, a polypeptide variant, or derivative thereof capable of performing the above enzymatic activity.

The term “isolated” as applied to the polynucleotide or polypeptide sequences disclosed herein is used to refer to sequences that are removed from their natural cellular environment. An isolated molecule may be obtained by any method or combination of methods including biochemical, recombinant, and synthetic techniques.

The term “recombinant” refers to a polynucleotide sequence that is removed from sequences that surround it in its natural context and/or is recombined with sequences that are not present in its natural context.

A “recombinant” polypeptide sequence is produced by translation from a “recombinant” polynucleotide sequence.

The term “derived from” with respect to polynucleotides or polypeptides of the invention being derived from a particular genera or species, means that the polynucleotide or polypeptide has the same sequence as a polynucleotide or polypeptide found naturally in that genera or species. The polynucleotide or polypeptide, derived from a particular genera or species, may therefore be produced synthetically or recombinantly.

### Variants

As used herein, the term “variant” refers to polynucleotide or polypeptide sequences different from the specifically identified sequences, wherein one or more nucleotides or amino acid residues is deleted, substituted, or added. Variants may be naturally occurring allelic variants, or non-naturally occurring variants. Variants may be from the same or from other species and may encompass homologues, paralogues and orthologues. In certain embodiments, variants of the inventive polypeptides and polypeptides possess biological activities that are the same or similar to those of the inventive polypeptides or polypeptides. The term “variant” with reference to polypeptides and polypeptides encompasses all forms of polypeptides and polypeptides as defined herein.

### Polynucleotide Variants

Variant polynucleotide sequences preferably exhibit at least 50%, more preferably at least 51%, more preferably at least 52%, more preferably at least 53%, more preferably at least 54%, more preferably at least 55%, more preferably at least 56%, more preferably at least 57%, more preferably at least 58%, more preferably at least 59%, more preferably at least 60%, more preferably at least 61%, more preferably at least 62%, more preferably at least 63%, more preferably at least 64%, more preferably at least 65%, more preferably at least 66%, more preferably at least 67%, more preferably at least 68%, more preferably at least 69%, more preferably at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a sequence of the present invention. Identity is found over a comparison window of at least 20 nucleotide positions, preferably at least 50 nucleotide positions, more preferably at least 100 nucleotide positions, and most preferably over the entire length of a polynucleotide of the invention.

Polynucleotide sequence identity can be determined in the following manner. The subject polynucleotide sequence is compared to a candidate polynucleotide sequence using BLASTN (from the BLAST suite of programs, version 2.2.5 [Nov. 2002]) in b12seq (Tatiana A. Tatusova, Thomas L. Madden (1999), “Blast 2 sequences-a new tool for comparing protein and nucleotide sequences”, FEMS Microbiol which Lett. 174:247-250), is publicly available from NCBI (ftp://ftp.ncbi.nih.gov/blast/). The default parameters of b12seq are utilized except that filtering of low complexity parts should be turned off.

The identity of polynucleotide sequences may be examined using the following unix command line parameters:
b12seq-i nucleotideseq1-j nucleotideseq2-F F-p blastn

The parameter-F F turns off filtering of low complexity sections. The parameter-p selects the appropriate algorithm for the pair of sequences. The b12seq program reports sequence identity as both the number and percentage of identical nucleotides in a line “Identities=”.

Polynucleotide sequence identity may also be calculated over the entire length of the overlap between a candidate and subject polynucleotide sequences using global sequence alignment programs (e.g. Needleman, S. B. and Wunsch, C. D. (1970) J. Mol. Biol. 48, 443-453). A full implementation of the Needleman-Wunsch global alignment algorithm is found in the needle program in the EMBOSS package (Rice, P. Longden, I. and Bleasby, A. EMBOSS: The European Molecular Biology Open Software Suite, Trends in Genetics June 2000, vol 16, No 6. pp. 276-277) which can be obtained from http://www.hgmp.mrc.ac.uk/Software/EMBOSS/. The European Bioinformatics Institute server also provides the facility to perform EMBOSS-needle global alignments between two sequences on line at http:/www.ebi.ac.uk/emboss/align/.

Alternatively the GAP program may be used which computes an optimal global alignment of two sequences without penalizing terminal gaps. GAP is described in the following paper: Huang, X. (1994) On Global Sequence Alignment. Computer Applications in the Biosciences 10, 227-235.

A preferred method for calculating polynucleotide % sequence identity is based on aligning sequences to be compared using Clustal X (Jeanmougin et al., 1998, Trends Biochem. Sci. 23, 403-5.)

Polynucleotide variants of the present invention also encompass those which exhibit a similarity to one or more of the specifically identified sequences that is likely to preserve the functional equivalence of those sequences and which could not reasonably be expected to have occurred by random chance. Such sequence similarity with respect to polypeptides may be determined using the publicly available b12seq program from the BLAST suite of programs (version 2.2.5 [Nov. 2002]) from NCBI (ftp://ftp.ncbi.nih.gov/blast/).

The similarity of polynucleotide sequences may be examined using the following unix command line parameters:
b12seq-i nucleotideseq1-j nucleotideseq2-F F-p tblastx

The parameter-F F turns off filtering of low complexity sections. The parameter-p selects the appropriate algorithm for the pair of sequences. This program finds regions of similarity between the sequences and for each such region reports an “E value” which is the expected number of times one could expect to see such a match by chance in a database of a fixed reference size containing random sequences. The size of this database is set by default in the b12seq program. For small E values, much less than one, the E value is approximately the probability of such a random match.

Variant polynucleotide sequences preferably exhibit an E value of less than 1×10−6 more preferably less than 1×10−9, more preferably less than 1×10−12, more preferably less than 1×10−15, more preferably less than 1×10−18, more preferably less than 1×10−21, more preferably less than 1×10−30, more preferably less than 1×10−40, more preferably less than 1×10−50, more preferably less than 1×10−60, more preferably less than 1×10−70, more preferably less than 1×10−80, more preferably less than 1×10−90 and most preferably less than 1×10−100 when compared with any one of the specifically identified sequences.

Alternatively, variant polynucleotides of the present invention, or used in the methods of the invention, hybridize to the specified polynucleotide sequences, or complements thereof under stringent conditions.

The term “hybridize under stringent conditions”, and grammatical equivalents thereof, refers to the ability of a polynucleotide molecule to hybridize to a target polynucleotide molecule (such as a target polynucleotide molecule immobilized on a DNA or RNA blot, such as a Southern blot or Northern blot) under defined conditions of temperature and salt concentration. The ability to hybridize under stringent hybridization conditions can be determined by initially hybridizing under less stringent conditions then increasing the stringency to the desired stringency.

With respect to polynucleotide molecules greater than about 100 bases in length, typical stringent hybridization conditions are no more than 25 to 30° C. (for example, 10° C.) below the melting temperature (Tm) of the native duplex (see generally, Sambrook et al., Eds, 1987, Molecular Cloning, A Laboratory Manual, 2nd Ed. Cold Spring Harbor Press; Ausubel et al., 1987, Current Protocols in Molecular Biology, Greene Publishing.). Tm for polynucleotide molecules greater than about 100 bases can be calculated by the formula Tm=81.5+0.41% (G+C-log (Na+). (Sambrook et al., Eds, 1987, Molecular Cloning, A Laboratory Manual, 2nd Ed. Cold Spring Harbor Press; Bolton and McCarthy, 1962, PNAS 84:1390). Typical stringent conditions for polynucleotide of greater than 100 bases in length would be hybridization conditions such as prewashing in a solution of 6×SSC. 0.2% SDS; hybridizing at 65° C., 6×SSC, 0.2% SDS overnight; followed by two washes of 30 minutes each in 1×SSC. 0.1% SDS at 65° C. and two washes of 30 minutes each in 0.2×SSC, 0.1% SDS at 65° C.

With respect to polynucleotide molecules having a length less than 100 bases, exemplary stringent hybridization conditions are 5 to 10° C. below Tm. On average, the Tm of a polynucleotide molecule of length less than 100 bp is reduced by approximately (500/oligonucleotide length® C.)

With respect to the DNA mimics known as peptide nucleic acids (PNAs) (Nielsen et al., Science. 1991 Dec. 6;254 (5037): 1497-500) Tm values are higher than those for DNA-DNA or DNA-RNA hybrids, and can be calculated using the formula described in Giesen et al., Nucleic Acids Res. 1998 November 1;26 (21): 5004-6. Exemplary stringent hybridization conditions for a DNA-PNA hybrid having a length less than 100 bases are 5 to 10° C. below the Tm.

Variant polynucleotides of the present invention, or used in the methods of the invention, also encompasses polynucleotides that differ from the sequences of the invention but that, as a consequence of the degeneracy of the genetic code, encode a polypeptide having similar activity to a polypeptide encoded by a polynucleotide of the present invention. A sequence alteration that does not change the amino acid sequence of the polypeptide is a “silent variation”. Except for ATG (methionine) and TGG (tryptophan), other codons for the same amino acid may be changed by art recognized techniques, e.g., to optimize codon expression in a particular host organism.

Polynucleotide sequence alterations resulting in conservative substitutions of one or several amino acids in the encoded polypeptide sequence without significantly altering its biological activity are also included in the invention. A skilled artisan will be aware of methods for making phenotypically silent amino acid substitutions (see, e.g., Bowie et al., 1990, Science 247, 1306).

Variant polynucleotides due to silent variations and conservative substitutions in the encoded polypeptide sequence may be determined using the publicly available b12seq program from the BLAST suite of programs (version 2.2.5 [Nov. 2002]) from NCBI (ftp://ftp.ncbi.nih.gov/blast/) via the tblastx algorithm as previously described.

### Polypeptide Variants

The term “variant” with reference to polypeptides encompasses naturally occurring, recombinantly and synthetically produced polypeptides. Variant polypeptide sequences preferably exhibit at least 50%, more preferably at least 51%, more preferably at least 52%, more preferably at least 53%, more preferably at least 54%, more preferably at least 55%, more preferably at least 56%, more preferably at least 57%, more preferably at least 58%, more preferably at least 59%, more preferably at least 60%, more preferably at least 61%, more preferably at least 62%, more preferably at least 63%, more preferably at least 64%, more preferably at least 65%, more preferably at least 66%, more preferably at least 67%, more preferably at least 68%, more preferably at least 69%, more preferably at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a sequences of the present invention. Identity is found over a comparison window of at least 20 amino acid positions, preferably at least 50 amino acid positions, more preferably at least 100 amino acid positions, and most preferably over the entire length of a polypeptide of the invention.

Polypeptide sequence identity can be determined in the following manner. The subject polypeptide sequence is compared to a candidate polypeptide sequence using BLASTP (from the BLAST suite of programs, version 2.2.5 [Nov. 2002]) in bl2seq, which is publicly available from NCBI (ftp://ftp.ncbi.nih.gov/blast/). The default parameters of b12seq are utilized except that filtering of low complexity regions should be turned off.

Polypeptide sequence identity may also be calculated over the entire length of the overlap between a candidate and subject polynucleotide sequences using global sequence alignment programs. EMBOSS-needle (available at http:/www.ebi.ac.uk/emboss/align/) and GAP (Huang, X. (1994) On Global Sequence Alignment. Computer Applications in the Biosciences 10, 227-235.) as discussed above are also suitable global sequence alignment programs for calculating polypeptide sequence identity.

A preferred method for calculating polypeptide % sequence identity is based on aligning sequences to be compared using Clustal X (Jeanmougin et al., 1998, Trends Biochem. Sci. 23, 403-5.)

Polypeptide variants of the present invention, or used in the methods of the invention, also encompass those which exhibit a similarity to one or more of the specifically identified sequences that is likely to preserve the functional equivalence of those sequences and which could not reasonably be expected to have occurred by random chance. Such sequence similarity with respect to polypeptides may be determined using the publicly available b12seq program from the BLAST suite of programs (version 2.2.5 [Nov. 2002]) from NCBI (ftp://ftp.ncbi.nih.gov/blast/). The similarity of polypeptide sequences may be examined using the following unix command line parameters:
b12seq-i peptideseq1-j peptideseq2-F F-p blastp

Variant polypeptide sequences preferably exhibit an E value of less than 1×10−6 more preferably less than 1×10−9, more preferably less than 1×10−12, more preferably less than 1×10−15, more preferably less than 1×10−18, more preferably less than 1×10−21, more preferably less than 1×10 −30, more preferably less than 1×10−40, more preferably less than 1×10−50, more preferably less than 1×10−60, more preferably less than 1×10−70, more preferably less than 1×10−80, more preferably less than 1×10−90 and most preferably 1×10−100 when compared with any one of the specifically identified sequences.

The parameter-F F turns off filtering of low complexity sections. The parameter-p selects the appropriate algorithm for the pair of sequences. This program finds regions of similarity between the sequences and for each such region reports an “E value” which is the expected number of times one could expect to see such a match by chance in a database of a fixed reference size containing random sequences. For small E values, much less than one, this is approximately the probability of such a random match.

Conservative substitutions of one or several amino acids of a described polypeptide sequence without significantly altering its biological activity are also included in the invention. A skilled artisan will be aware of methods for making phenotypically silent amino acid substitutions (see, e.g., Bowie et al., 1990, Science 247, 1306).

### Constructs, Vectors and Components Thereof

The term “genetic construct” refers to a polynucleotide molecule, usually double-stranded DNA, which may have inserted into it another polynucleotide molecule (the insert polynucleotide molecule) such as, but not limited to, a cDNA molecule. A genetic construct may contain the necessary elements that permit transcribing the insert polynucleotide molecule, and, optionally, translating the transcript into a polypeptide. The insert polynucleotide molecule may be derived from the host cell, or may be derived from a different cell or organism and/or may be a recombinant polynucleotide. Once inside the host cell the genetic construct may become integrated in the host chromosomal DNA. The genetic construct may be linked to a vector.

The term “vector” refers to a polynucleotide molecule, usually double stranded DNA, which is used to transport the genetic construct into a host cell. The vector may be capable of replication in at least one additional host system, such as E. coli.

The term “expression construct” refers to a genetic construct that includes the necessary elements that permit transcribing the insert polynucleotide molecule, and, optionally, translating the transcript into a polypeptide. An expression construct typically comprises in a 5′ to 3′ direction:
a) a promoter functional in the host cell into which the construct will be transformed,
b) the polynucleotide to be expressed, and
c) a terminator functional in the host cell into which the construct will be transformed.

The term “coding region” or “open reading frame” (ORF) refers to the sense strand of a genomic DNA sequence or a cDNA sequence that is capable of producing a transcription product and/or a polypeptide under the control of appropriate regulatory sequences. The coding sequence may, in some cases, identified by the presence of a 5′ translation start codon and a 3′ translation stop codon. When inserted into a genetic construct, a “coding sequence” is capable of being expressed when it is operably linked to promoter and terminator sequences.

“Operably-linked” means that the sequenced to be expressed is placed under the control of regulatory elements that include promoters, tissue-specific regulatory elements, temporal regulatory elements, enhancers, repressors and terminators.

The term “noncoding region” refers to untranslated sequences that are upstream of the translational start site and downstream of the translational stop site. These sequences are also referred to respectively as the 5′ UTR and the 3′ UTR. These regions include elements required for transcription initiation and termination, mRNA stability, and for regulation of translation efficiency.

Terminators are sequences, which terminate transcription, and are found in the 3′ untranslated ends of genes downstream of the translated sequence. Terminators are important determinants of mRNA stability and in some cases have been found to have spatial regulatory functions.

The term “promoter” refers to nontranscribed cis-regulatory elements upstream of the coding region that regulate gene transcription. Promoters comprise cis-initiator elements which specify the transcription initiation site and conserved boxes such as the TATA box, and motifs that are bound by transcription factors. Introns within coding sequences can also regulate transcription and influence post-transcriptional processing (including splicing, capping and polyadenylation).

A promoter may be homologous with respect to the polynucleotide to be expressed. This means that the promoter and polynucleotide are found operably linked in nature.

Alternatively the promoter may be heterologous with respect to the polynucleotide to be expressed. This means that the promoter and the polynucleotide are not found operably linked in nature.

A “transgene” is a polynucleotide that is taken from one organism and introduced into a different organism by transformation. The transgene may be derived from the same species or from a different species as the species of the organism into which the transgene is introduced.

An “inverted repeat” is a sequence that is repeated, where the second half of the repeat is in the complementary strand, e.g.,

(5′)GATCTA . . . TAGATC(3′)
(3′)CTAGAT . . . ATCTAG(5′)

Read-through transcription will produce a transcript that undergoes complementary base-pairing to form a hairpin structure provided that there is a 3-5 bp spacer between the repeated regions.

### Host Cells

Host cells may be derived from, for example, bacterial, fungal, yeast, insect, mammalian, algal or plant organisms. Host cells may also be synthetic cells. Preferred host cells are eukaryotic cells. A particularly preferred host cell is a plant cell.

A “transgenic plant” refers to a plant which contains new genetic material as a result of genetic manipulation or transformation. The new genetic material may be derived from a plant of the same species as the resulting transgenic plant or from a different species.

### Methods for Isolating or Producing Polynucleotides

The polynucleotide molecules of the invention can be isolated by using a variety of techniques known to those of ordinary skill in the art. By way of example, such polypeptides can be isolated through use of the polymerase chain reaction (PCR) described in Mullis et al., Eds. 1994 The Polymerase Chain Reaction, Birkhauser, incorporated herein by reference. The polypeptides of the invention can be amplified using primers, as defined herein, derived from the polynucleotide sequences of the invention.

Further methods for isolating polynucleotides of the invention include use of all, or portions of, the polypeptides having the sequence set forth herein as hybridization probes. The technique of hybridizing labelled polynucleotide probes to polynucleotides immobilized on solid supports such as nitrocellulose filters or nylon membranes, can be used to screen the genomic or cDNA libraries. Exemplary hybridization and wash conditions are: hybridization for 20 hours at 65° C. in 5.0×SSC, 0.5% sodium dodecyl sulfate, 1×Denhardt's solution; washing (three washes of twenty minutes each at 55° C.) in 1.0×SSC, 1% (w/v) sodium dodecyl sulfate, and optionally one wash (for twenty minutes) in 0.5×SSC, 1% (w/v) sodium dodecyl sulfate, at 60° C. An optional further wash (for twenty minutes) can be conducted under conditions of 0.1×SSC, 1% (w/v) sodium dodecyl sulfate, at 60° C.

The polynucleotide fragments of the invention may be produced by techniques well-known in the art such as restriction endonuclease digestion, oligonucleotide synthesis and PCR amplification.

A partial polynucleotide sequence may be used, in methods well-known in the art to identify the corresponding full length polynucleotide sequence. Such methods include PCR-based methods, 5′RACE (Frohman MA, 1993, Methods Enzymol. 218:340-56) and hybridization-based method, computer/database-based methods. Further, by way of example, inverse PCR permits acquisition of unknown sequences, flanking the polynucleotide sequences disclosed herein, starting with primers based on a known region (Triglia et al., 1998, Nucleic Acids Res 16, 8186, incorporated herein by reference). The method uses several restriction enzymes to generate a suitable fragment in the known region of a gene. The fragment is then circularized by intramolecular ligation and used as a PCR template. Divergent primers are designed from the known region. In order to physically assemble full-length clones, standard molecular biology approaches can be utilized (Sambrook et al., Molecular Cloning: A Laboratory Manual, 2nd Ed. Cold Spring Harbor Press, 1987).

It may be beneficial, when producing a transgenic plant from a particular species, to transform such a plant with a sequence or sequences derived from that species. The benefit may be to alleviate public concerns regarding cross-species transformation in generating transgenic organisms. Additionally when down-regulation of a gene is the desired result, it may be necessary to utilise a sequence identical (or at least highly similar) to that in the plant, for which reduced expression is desired. For these reasons among others, it is desirable to be able to identify and isolate orthologues of a particular gene in several different plant species.

Variants (including orthologues) may be identified by the methods described.

### Methods for Identifying Variants

### Physical Methods

Variant polypeptides may be identified using PCR-based methods (Mullis et al., Eds. 1994 The Polymerase Chain Reaction, Birkhauser). Typically, the polynucleotide sequence of a primer, useful to amplify variants of polynucleotide molecules of the invention by PCR, may be based on a sequence encoding a conserved region of the corresponding amino acid sequence.

Alternatively library screening methods, well known to those skilled in the art, may be employed (Sambrook et al., Molecular Cloning: A Laboratory Manual, 2nd Ed. Cold Spring Harbor Press, 1987). When identifying variants of the probe sequence, hybridization and/or wash stringency will typically be reduced relatively to when exact sequence matches are sought.

Polypeptide variants may also be identified by physical methods, for example by screening expression libraries using antibodies raised against polypeptides of the invention (Sambrook et al., Molecular Cloning: A Laboratory Manual, 2nd Ed. Cold Spring Harbor Press, 1987) or by identifying polypeptides from natural sources with the aid of such antibodies.

### Computer Based Methods

The variant sequences of the invention, including both polynucleotide and polypeptide variants, may also be identified by computer-based methods well-known to those skilled in the art, using public domain sequence alignment algorithms and sequence similarity search tools to search sequence databases (public domain databases include Genbank, EMBL, Swiss-Prot, PIR and others). See, e.g., Nucleic Acids Res. 29:1-10 and 11-16, 2001 for examples of online resources. Similarity searches retrieve and align target sequences for comparison with a sequence to be analyzed (i.e., a query sequence). Sequence comparison algorithms use scoring matrices to assign an overall score to each of the alignments.

An exemplary family of programs useful for identifying variants in sequence databases is the BLAST suite of programs (version 2.2.5 [Nov. 2002]) including BLASTN, BLASTP, BLASTX, tBLASTN and tBLASTX, which are publicly available from (ftp://ftp.ncbi.nih.gov/blast/) or from the National Center for Biotechnology Information (NCBI), National Library of Medicine, Building 38A, Room 8N₈₀₅, Bethesda, MD 20894 USA. The NCBI server also provides the facility to use the programs to screen a number of publicly available sequence databases. BLASTN compares a nucleotide query sequence against a nucleotide sequence database. BLASTP compares an amino acid query sequence against a protein sequence database. BLASTX compares a nucleotide query sequence translated in all reading frames against a protein sequence database. tBLASTN compares a protein query sequence against a nucleotide sequence database dynamically translated in all reading frames. tBLASTX compares the six-frame translations of a nucleotide query sequence against the six-frame translations of a nucleotide sequence database. The BLAST programs may be used with default parameters or the parameters may be altered as required to refine the screen.

The use of the BLAST family of algorithms, including BLASTN, BLASTP, and BLASTX, is described in the publication of Altschul et al., Nucleic Acids Res. 25:3389-3402, 1997.

The “hits” to one or more database sequences by a queried sequence produced by BLASTN. BLASTP, BLASTX, tBLASTN, tBLASTX, or a similar algorithm, align and identify similar portions of sequences. The hits are arranged in order of the degree of similarity and the length of sequence overlap. Hits to a database sequence generally represent an overlap over only a fraction of the sequence length of the queried sequence.

The BLASTN, BLASTP, BLASTX, tBLASTN and tBLASTX algorithms also produce “Expect” values for alignments. The Expect value (E) indicates the number of hits one can “expect” to see by chance when searching a database of the same size containing random contiguous sequences. The Expect value is used as a significance threshold for determining whether the hit to a database indicates true similarity. For example, an E value of 0.1 assigned to a polynucleotide hit is interpreted as meaning that in a database of the size of the database screened, one might expect to see 0.1 matches over the aligned portion of the sequence with a similar score simply by chance. For sequences having an E value of 0.01 or less over aligned and matched portions, the probability of finding a match by chance in that database is 1% or less using the BLASTN, BLASTP, BLASTX, tBLASTN or tBLASTX algorithm.

Multiple sequence alignments of a group of related sequences can be carried out with CLUSTALW (Thompson, J. D., Higgins, D. G. and Gibson, T. J. (1994) CLUSTALW: improving the sensitivity of progressive multiple sequence alignment through sequence weighting, positions-specific gap penalties and weight matrix choice. Nucleic Acids Research, 22:4673-4680, http://www-igbmc.u-strasbg.fr/BioInfo/ClustalW/Top.html) or T-COFFEE (Cedric Notredame, Desmond G. Higgins, Jaap Heringa, T-Coffee: A novel method for fast and accurate multiple sequence alignment, J. Mol. Biol. (2000) 302:205-217)) or PILEUP, which uses progressive, pairwise alignments. (Feng and Doolittle, 1987, J. Mol. Evol. 25, 351).

Pattern recognition software applications are available for finding motifs or signature sequences. For example, MEME (Multiple Em for Motif Elicitation) finds motifs and signature sequences in a set of sequences, and MAST (Motif Alignment and Search Tool) uses these motifs to identify similar or the same motifs in query sequences. The MAST results are provided as a series of alignments with appropriate statistical data and a visual overview of the motifs found. MEME and MAST were developed at the University of California, San Diego.

PROSITE (Bairoch and Bucher, 1994, Nucleic Acids Res. 22, 3583; Hofmann et al., 1999, Nucleic Acids Res. 27, 215) is a method of identifying the functions of uncharacterized proteins translated from genomic or cDNA sequences. The PROSITE database (www.expasy.org/prosite) contains biologically significant patterns and profiles and is designed so that it can be used with appropriate computational tools to assign a new sequence to a known family of proteins or to determine which known domain(s) are present in the sequence (Falquet et al., 2002, Nucleic Acids Res. 30, 235). Prosearch is a tool that can search SWISS-PROT and EMBL databases with a given sequence pattern or signature.

### Methods for Isolating Polypeptides

The polypeptides of the invention, or used in the methods of the invention, including variant polypeptides, may be prepared using peptide synthesis methods well known in the art such as direct peptide synthesis using solid phase techniques (e.g. Stewart et al., 1969, in Solid-Phase Peptide Synthesis, WH Freeman Co, San Francisco California, or automated synthesis, for example using an Applied Biosystems 431A Peptide Synthesizer (Foster City, California). Mutated forms of the polypeptides may also be produced during such syntheses.

The polypeptides and variant polypeptides of the invention, or used in the methods of the invention, may also be purified from natural sources using a variety of techniques that are well known in the art (e.g. Deutscher, 1990, Ed, Methods in Enzymology, Vol. 182, Guide to Protein Purification,).

Alternatively the polypeptides and variant polypeptides of the invention, or used in the methods of the invention, may be expressed recombinantly in suitable host cells and separated from the cells as discussed below.

### Methods for Producing Constructs and Vectors

The genetic constructs of the present invention comprise one or more polynucleotide sequences of the invention and/or polynucleotides encoding polypeptides of the invention, and may be useful for transforming, for example, bacterial, fungal, insect, mammalian or plant organisms. The genetic constructs of the invention are intended to include expression constructs as herein defined.

Methods for producing and using genetic constructs and vectors are well known in the art and are described generally in Sambrook et al., Molecular Cloning: A Laboratory Manual, 2nd Ed. Cold Spring Harbor Press, 1987; Ausubel et al., Current Protocols in Molecular Biology, Greene Publishing, 1987).

### Methods for Producing Host Cells Comprising Polynucleotides, Constructs or Vectors

The invention provides a host cell which comprises a genetic construct or vector of the invention.

Host cells comprising genetic constructs, such as expression constructs, of the invention are useful in methods well known in the art (e.g. Sambrook et al., Molecular Cloning: A Laboratory Manual, 2nd Ed. Cold Spring Harbor Press, 1987; Ausubel et al., Current Protocols in Molecular Biology, Greene Publishing, 1987) for recombinant production of polypeptides of the invention. Such methods may involve the culture of host cells in an appropriate medium in conditions suitable for or conducive to expression of a polypeptide of the invention. The expressed recombinant polypeptide, which may optionally be secreted into the culture, may then be separated from the medium, host cells or culture medium by methods well known in the art (e.g. Deutscher, Ed, 1990, Methods in Enzymology, Vol 182, Guide to Protein Purification).

### Methods for Producing Plant Cells and Plants Comprising Constructs and Vectors

The invention further provides plant cells which comprise a genetic construct of the invention, and plant cells modified to alter expression of a polynucleotide or polypeptide of the invention, or used in the methods of the invention. Plants comprising such cells also form an aspect of the invention.

Methods for transforming plant cells, plants and portions thereof with polypeptides are described in Draper et al., 1988, Plant Genetic Transformation and Gene Expression. A Laboratory Manual. Blackwell Sci. Pub. Oxford, p. 365; Potrykus and Spangenburg, 1995, Gene Transfer to Plants. Springer-Verlag, Berlin.; and Gelvin et al., 1993, Plant Molecular Biol. Manual. Kluwer Acad. Pub. Dordrecht. A review of transgenic plants, including transformation techniques, is provided in Galun and Breiman, 1997, Transgenic Plants. Imperial College Press, London.

### Methods for Genetic Manipulation of Plants

A number of plant transformation strategies are available (e.g. Birch, 1997, Ann Rev Plant Phys Plant Mol Biol, 48, 297, Hellens R P, et al (2000) Plant Mol Biol 42:819-32, Hellens R et al Plant Meth 1: 13). For example, strategies may be designed to increase expression of a polynucleotide/polypeptide in a plant cell, organ and/or at a particular developmental stage where/when it is normally expressed or to ectopically express a polynucleotide/polypeptide in a cell, tissue, organ and/or at a particular developmental stage which/when it is not normally expressed. The expressed polynucleotide/polypeptide may be derived from the plant species to be transformed or may be derived from a different plant species.

Transformation strategies may be designed to reduce expression of a polynucleotide/polypeptide in a plant cell, tissue, organ or at a particular developmental stage which/when it is normally expressed. Such strategies are known as gene silencing strategies.

Genetic constructs for expression of genes in transgenic plants typically include promoters for driving the expression of one or more cloned polynucleotide, terminators and selectable marker sequences to detect presence of the genetic construct in the transformed plant.

The promoters suitable for use in the constructs of this invention are functional in a cell, tissue or organ of a monocot or dicot plant and include cell-, tissue- and organ-specific promoters, cell cycle specific promoters, temporal promoters, inducible promoters, constitutive promoters that are active in most plant tissues, and recombinant promoters. Choice of promoter will depend upon the temporal and spatial expression of the cloned polynucleotide, so desired. The promoters may be those normally associated with a transgene of interest, or promoters which are derived from genes of other plants, viruses, and plant pathogenic bacteria and fungi. Those skilled in the art will, without undue experimentation, be able to select promoters that are suitable for use in modifying and modulating plant traits using genetic constructs comprising the polynucleotide sequences of the invention. Examples of constitutive plant promoters include the CaMV 35S promoter, the nopaline synthase promoter and the octopine synthase promoter, and the Ubi 1 promoter from maize. Plant promoters which are active in specific tissues, respond to internal developmental signals or external abiotic or biotic stresses are described in the scientific literature. Exemplary promoters are described, e.g., in WO 02/00894, which is herein incorporated by reference.

Exemplary terminators that are commonly used in plant transformation genetic construct include, e.g., the cauliflower mosaic virus (CaMV) 35S terminator, the Agrobacterium tumefaciens nopaline synthase or octopine synthase terminators, the Zea mays zein gene terminator, the Oryza sativa ADP-glucose pyrophosphorylase terminator and the Solanum tuberosum PI-II terminator.

Selectable markers commonly used in plant transformation include the neomycin phophotransferase II gene (NPT II) which confers kanamycin resistance, the aadA gene, which confers spectinomycin and streptomycin resistance, the phosphinothricin acetyl transferase (bar gene) for Ignite (AgrEvo) and Basta (Hoechst) resistance, and the hygromycin phosphotransferase gene (hpt) for hygromycin resistance.

Those skilled in the art will understand that polynucleotides and constructs for expressing polypeptides in cells and plants can include various other modifications including restriction sites, recombination/excision sites, codon optomisiation, tags to facilitate protein purification, etc. Those skilled in the art will understand how to utilise such modifications, some of which may influence transgene expression, stability and translation. However, an art skilled worker would also understand that these modifications are not essential, and do not limit the scope of the invention.

The following are representative publications disclosing genetic transformation protocols that can be used to genetically transform the following plant species: Rice (Alam et al., 1999, Plant Cell Rep. 18, 572); apple (Yao et al., 1995, Plant Cell Reports 14, 407-412); maize (U.S. Pat. Nos. 5,177,010 and 5,981,840); wheat (Ortiz et al., 1996, Plant Cell Rep. 15, 1996, 877); tomato (U.S. Pat. No. 5,159,135); potato (Kumar et al., 1996 Plant J. 9, 821); cassava (Li et al., 1996 Nat. Biotechnology 14, 736); lettuce (Michelmore et al., 1987, Plant Cell Rep. 6, 439); tobacco (Horsch et al., 1985, Science 227, 1229); cotton (U.S. Pat. Nos. 5,846,797 and 5,004,863); grasses (U.S. Pat. Nos. 5,187,073 and 6,020,539); peppermint (Niu et al., 1998, Plant Cell Rep. 17, 165); citrus plants (Pena et al., 1995, Plant Sci.104, 183); caraway (Krens et al., 1997, Plant Cell Rep, 17, 39); banana (U.S. Pat. No. 5,792,935); soybean (U.S. Pat. Nos. 5,416,011; 5,569,834; 5,824,877; 5,563,04455 and 5,968,830); pineapple (U.S. Pat. No. 5,952,543); poplar (U.S. Pat. No. 4,795,855); monocots in general (U.S. Pat. Nos. 5,591,616 and 6,037,522); brassica (U.S. Pat. Nos. 5,188,958; 5,463,174 and 5,750,871); cereals (U.S. Pat. No. 6,074,877); pear (Matsuda et al., 2005, Plant Cell Rep. 24 (1): 45-51); Prunus (Ramesh et al., 2006 Plant Cell Rep. 25 (8): 821-8; Song and Sink 2005 Plant Cell Rep. 2006; 25 (2): 117-23; Gonzalez Padilla et al., 2003 Plant Cell Rep.22 (1): 38-45); strawberry (Oosumi et al., 2006 Planta. 223 (6): 1219-30; Folta et al., 2006 Planta April 14; PMID: 16614818), rose (Li et al., 2003), Rubus (Graham et al., 1995 Methods Mol Biol. 1995; 44:129-33), tomato (Dan et al., 2006, Plant Cell Reports V25: 432-441), apple (Yao et al., 1995, Plant Cell Rep. 14, 407-412), Canola (Brassica napus L.). (Cardoza and Stewart, 2006 Methods Mol Biol. 343:257-66), safflower (Orlikowska et al, 1995, Plant Cell Tissue and Organ Culture 40:85-91), ryegrass (Altpeter et al, 2004 Developments in Plant Breeding 11 (7): 255-250), rice (Christou et al, 1991 Nature Biotech. 9:957-962), maize (Wang et al 2009 In: Handbook of Maize pp. 609-639) and Actinidia eriantha (Wang et al., 2006, Plant Cell Rep. 25, 5:425-31). Transformation of other species is also contemplated by the invention. Suitable methods and protocols are available in the scientific literature.

### Modification of Endogenous Genomes

Targeted genome editing using engineered nucleases such as clustered, regularly interspaced, short palindromic repeat (CRISPR) technology, is an important new approach for generating RNA-guided nucleases, such as Cas9, with customizable specificities. Genome editing mediated by these nucleases has been used to rapidly, easily and efficiently modify endogenous genes in a wide variety of cell types and in organisms that have traditionally been challenging to manipulate genetically. A modified version of the CRISPR-Cas9 system has been developed to recruit heterologous domains that can regulate endogenous gene expression or label specific genomic loci in living cells (Nature Biotechnology 32, 347-355 (2014). The system is applicable to plants, and can be used to regulate expression of target genes. (Bortesi and Fischer, Biotechnology Advances Volume 33, Issue 1, January-February 2015, Pages 41-52). Use of CRISPR technology in plants is also reviewed in Zhang et al., 2019, Nature Plants, Volume 5, pages778-794.

### Plants

The term “plant” is intended to include a whole plant, any part of a plant, a seed, a fruit, propagules and progeny of a plant.

The term ‘propagule’ means any part of a plant that may be used in reproduction or propagation, either sexual or asexual, including seeds and cuttings.

The plants of the invention may be grown and either self-ed or crossed with a different plant strain and the resulting hybrids, with the desired phenotypic characteristics, may be identified. Two or more generations may be grown to ensure that the subject phenotypic characteristics are stably maintained and inherited. Plants resulting from such standard breeding approaches also form an aspect of the present invention.

### BRIEF DESCRIPTION OF THE FIGURES

FIG. 1 shows net leaf dry matter accumulated over the course of the experiment.

FIG. 2 shows leaf biomass from each harvest, 0 kg N/ha treatment.

FIG. 3 shows leaf biomass from each harvest, 400 kg N/ha treatment.

FIG. 4 shows eaf biomass from each harvest, 800 kg N/ha treatment.

FIG. 5 shows nitrous oxide flux, 0 and 400 kg N/ha treatment, ODR6205 and IMP566 clones.

FIG. 6 shows nitrous oxide flux, 0 and 800 kg N/ha treatment, ODR6205 and IMP566 clones.

FIG. 7 shows total N₂O emitted (mg N m⁻²) over 90 days post artificial urine application, ODR6205 and IMP566 clones.

FIG. 8 shows nitrous oxide emission factor (EF) over 90 days post artificial urine application, ODR6205 and IMP566 clones.

FIG. 9 shows nitrous oxide flux, 400 kg N/ha (as urine), ODR6205 and IMP566 clones.

FIG. 10 shows nitrous oxide flux, 400 kg N/ha (as urine), RCR5101 and Null populations.

### EXAMPLES

This invention will now be illustrated with reference to the following non-limiting examples.

### Example 1: Construct Designs

The Garden Nasturtium (Tropaeolum majus) DGAT1 peptide sequence (GenBank AAM03340) with the single point mutation of serine at 197 amino acid sequence to alanine as described by Xu et al. (2008),-SEQ ID NO: 26, linked with V5 epitope tag (GKPIPNPLLGLDST) at the C-terminal (DGAT1-V5), and the 15-kD sesame L-oleosin (accession no. AAD42942) with three engineered cysteine residues on each N- and C-terminal amphipathic arms (Cys-OLE; Winichayakul et al., 2013-SEQ ID NO: 49) were custom synthesized by GeneART™ for expression in L. perenne (sequences 1-4) Both DGAT1-V5 and Cys-OLE coding sequences were optimized for expression in monocot grass. For Agrobacterium-mediated transformation the expression cassette was cloned into the pCAMBIA1300 binary vector while for particle bombardment the cassette was cloned into a pUC-based vector.

The resulting constructs, labelled as LpDlo3-3, contained the DGAT1-V5 gene regulated by the rice ribulose-1, 5-bisphosphate carboxylase small subunit promoter (RuBisCO-Sp, GenBank AY583764) back-to-back with the Cys-OLE gene regulated by the rice chlorophyll a/b binding protein promoter (CABp; GenBank AP014965-region: 10845004-10845835).

Transformed lines were generated from Lolium perenne callus induced from immature inflorescences and transformed by Agrobacterium-mediated transformation or particle bombardment. Plants generated from Agrobacterium-mediated transformation were generated as per Bajaj et al. (2006) while plants from microprojectile bombardment the method described by Altpeter et al. (2000).
Phaseolus vulgaris ribulose 1,5-bisphosphate carboxylase/oxygenase small subunit (rbcS2) promoter, Accession number AF028707
Pisum sativum small subunit ribulose bisphosphate carboxylase (rbcS-3A) promoter, Accession numbers M21356; M27973
Pisum sativum CAB promoter, Accession number M64619
Glycine max Subunit-1 ubiquitin promoter, Accession number D16248
Arabidopsis thaliana polyubiquitin 10 promoter, Accession number L05399
Cauliflower mosaic virus 35s promoter, Accession numbers V00141; J02048

These were subcloned into binary vectors for Agrobacterium tumefaciens assisted transformation.

The same peptide sequences were optimized for expression in Cannabis sativa (sequences 9-12) and were placed under a variety of promoter combinations including but not limited to:
Phaseolus vulgaris ribulose 1,5-bisphosphate carboxylase/oxygenase small subunit (rbcS2) promoter, Accession number AF028707
Pisum sativum small subunit ribulose bisphosphate carboxylase (rbcS-3A) promoter, Accession numbers M21356; M27973.
Pisum sativum CAB promoter, Accession number M64619
Glycine max Subunit-1 ubiquitin promoter, Accession number D16248
Arabidopsis thaliana polyubiquitin 10 promoter, Accession number L05399
Cauliflower mosaic virus 35s promoter, Accession numbers V00141; J02048

These were subcloned into binary vectors for Agrobacterium tumefaciens assisted transformation.

### Example 2: Lolium perenne Transformation, Selection and Growth Conditions

Plants over-expressing the LpD103-3 construct were generated by Agrobacterium-mediated transformation as per Bajaj et al. (2006) and by microprojectile bombardment using a method adapted from Altpeter et al. (2000). Briefly, calli for microprojectile bombardment transformation were induced from immature inflorescences harvested from a single transformation-competent genotype of cvr. Impact (IMP566) by culture on a Murashige Skoog basal medium supplemented with 2,4-dichlorophenoxyacetic acid. Plasmids for transformation were prepared using the Invitrogen Pure Link Hi Pure Plasmid Maxiprep Kit. The plasmid pAcH1, which contains an expression cassette comprising a chimeric hygromycin phosphotransferase (HPH) gene (Bilang et al., 1991) expressed from the rice actin promoter, was used for selection and mixed in a 1:1 molar ratio with LpDlo3-3. Plasmid DNA's were coated onto M17 tungsten particles using the method of Sanford et al. (1993) and co-transformed into target tissues using a DuPont PDS-1000/He Biolistic Particle Delivery System. Multiple independent heterozygous ryegrass transformants were generated, including transgenic plants transformed with pAcH1 as a vector control (VC). Transformed plants were transferred to a contained greenhouse environment (22/17° C. diurnal cycle and 12 hour photoperiod under supplementary LED lighting providing 1000 u M/see/m2 PAR) for further analysis. Transformation by Agrobacterium was performed as per Bajaj et al. (2006) using the same stage immature inforescenses as described above for the microprojectile bombardment except that the cultivar used was Alto (Alto100).

PCR analysis using primer pair's specific to the HPH and DGAT genes was performed to confirm stable integration of the transgenes into the genome of plants recovered from transformation experiments, and Southern blot hybridization was used to estimate the number of transgene copies per line. Leaves from these plants were initially analysed for total fatty acid content and recombinant DGAT1-V5 and Cys-OLE proteins.

The transgenic Lolium perenne line generated by microprojectile bombardment used was ODR6205; the control for this line was Wild Type (WT) IMP566. In both cases (ODR6205 and IMP566) clonal splits were used to propagate sufficient replicate material for the experimental work; as such all ODR6205 material described here was clonal To generation. The transgenic Lolium perenne line generated by Agrobacterium mediated transformation used was homozygous RCR5101; the control for this line was RCR5101 null plants. The generation of homozygous and null plants is described below.

### Example 3: Generation of Homozygous and Null RCR5101 Populations

Agrobacterium generated To HME plants were phenotyped by testing for the presence of cysteine-oleosin (immunoblot) and measuring the leaf FA content (FAMES GC-FID). Genotyping data for single copy lines RCR5101 showed the HME T-DNA was a single locus.

The RCR5101 T₀ plant was pair crossed with three independent founder donor plants to generate families of T₁ seed. Approximately 50 T₁ seeds from each successful cross were germinated in controlled growth rooms, the seedlings were fed with Thrive™ on a weekly basis, watered when necessary and rotated daily. A 3 cm piece of leaf was removed from each plant and analysed by dot blot for the presence of cysteine-oleosin. One tiller was removed from each plant that tested cysteine-oleosin positive and analysed for AR37 endophyte either by immunoblot or by qPCR. Total leaf material was harvested approximately 5 cm above the pots from all plants; this was freeze dried, ground and analysed by FAMES GC-FID.

Four T1 hemizygous plants from each family were chosen to go forward to generate T2 seed. Essentially this included the plants with the highest level of leaf FA as well as a spare line. In each case the plants were visually inspected for growth habit and relative size comparison against the other plants.

Seeds were then generated by crossing 3 full T1 siblings within a single cage. These crosses could be expected to produce T2 null: hemizygous: homozygous progeny at a 1:2:1 ratio respectively. Phenotyping was performed on approximately 200 seeds from each of the full sibling crosses; similarly, each seedling was genotyped using digital drop PCR (ddPCR) which identified null, hemizygous and homozygous plants. An example of the gene dosage effect on the leaf fatty acid in four RCR5101 families generated by full sib crosses is shown in Table 3.

TABLE 3

| | | | | | | INC % FA | GENE |
|---|---|---|---|---|---|---|---|
| | | 18:2/18:3 | SD | % FA | SD | OVER NULL | DOSAGE |
| RCR5101 full sib | NULL | 0.1349 | 0.01 | 4.4321 | 0.18 | | |
| parent 69809-5 | HEMI | 0.2824 | 0.04 | 5.6305 | 0.38 | 1.1984 | |
| clone 2619 | HOMO | 0.3901 | 0.03 | 6.8489 | 0.56 | 2.4169 | 2.02 |
| RCR5101 full sib | NULL | 0.1413 | 0.01 | 4.3951 | 0.12 | | |
| parent 69809-5 | HEMI | 0.2769 | 0.04 | 5.6453 | 0.43 | 1.2503 | |
| clone 2610 | HOMO | 0.4052 | 0.02 | 6.9620 | 0.25 | 2.5669 | 2.05 |
| RCR5101 full sib | NULL | 0.1377 | 0.01 | 4.4114 | 0.20 | | |
| parent 69808-3 | HEMI | 0.2720 | 0.03 | 5.4498 | 0.35 | 1.0384 | |
| clone 2611 | HOMO | 0.3835 | 0.01 | 6.6455 | 0.09 | 2.2341 | 2.15 |
| RCR5101 full sib | NULL | 0.1433 | 0.01 | 4.4358 | 0.21 | | |
| parent 69808-3 | HEMI | 0.2663 | 0.01 | 5.5760 | 0.42 | 1.1402 | |
| clone 2631 | HOMO | 0.3931 | 0.03 | 6.6560 | 0.20 | 2.2202 | 1.95 |

Twelve individual T2 rapid homozygous plants from three separate founder plant families (36 plants in total) were selected based on high % leaf fatty acid, high C18: 2/C18: 3 ratio and the presence of cysteine-oleosin. The plants were cloned into three copies (two for use in crossing and one back up plant). To produce a relatively diverse population of T3 seed 2 copies of 1 founder background, 2 copies of a second founder background and 2 copies of the third founder background were randomised in controlled cage crosses to produce a total of 12 populations of T3 homozygous seed. Similarly, the same crossing procedure was also performed with T2 nulls. Plants germinated from the T3 seeds were used in the determination of N₂O emissions after treatment with bovine urine.

### Example 4: Glycine max Transformation

Glycine max can be transformed and selected essentially as described in Zeng, P. et al 2004, Plant Cell Reports, 22:478-482, and Paz N, M. et al., 2004, Euphytica, 136:167-179.

### Example 5: Cannabis sativa Transformation

Cannabis sativa can be transformed and selected essentially as described in Feeney and Punja (2003).

### Example 6: Reduced N₂O Emissions from the Soil (after Application of Artificial Urine) in which ODR6205 Plants Produced by Methods of the Invention are Grown

### Experimental Design

The N₂O experiment was carried out in mesocosms in a controlled temperature room (12-h day-light, 20° C. day and 15° C. night temperature) with LED lighting (California Lightworks SolarSystem 550 Commercial Series) giving approximately 700 μmol m⁻²s⁻¹ light at the plant height.

Two different perennial ryegrass plant genotypes were used, a T₀ perennial ryegrass plant line (ODR6205) and its wild type parent (Impact, Imp566).

Each plant line was subjected to two different urine N loadings (equivalent to a moderate and high N rate dairy cow urine patch) was tested. A zero-N treatment was also included. The N loads, 400N and 800N (approximately equivalent to 400 kg N/ha and 800 kg N/ha respectively), were delivered as artificial urine applied at a common volume (a standard dairy urination event of 10 L m⁻²) with the N concentration varying to deliver each N load.

As such, the experimental design was a row column design with 36 mesocosms (2 plant genotypes ×3 N loads, with 6 replicates per N load and plant line) placed in 4 rows of 9 mesocosms each.

The response variables measured included: leaf dry matter (DM) harvested; cumulative N₂O emissions; and N₂O emission factor (EF) expressed as the % of applied N emitted as N₂O.

### Mesocosm Set Up

Mesocosms were constructed of 190 mm diameter PVC drainpipe cut into 300 mm lengths. Wire mesh discs covered with a layer of frost cloth were placed 20 mm from the bottom of the mesocosm to provide a base for the soil and allow for drainage.

Eight weeks prior to transplanting, 36 mesocosms were filled with soil collected from a paddock previously grazed by sheep. The top 20-30 mm of sod was removed and soil to 200 mm depth was removed, thoroughly mixed and any roots and stones removed. Mesocosms were filled with approximately 10 kg field moist soil each and packed to a bulk density of ˜0.8 g cm³, leaving a 20 mm gap from the soil surface to the top of the mesocosm, giving a 260 mm soil column.

The soil was classified as a Karapoti silt loam (Dystric Eutrochrept) and at the time of collection the soil characteristics were; pH 5.4 (1:2.1 V/V water slurry: electrode determination); carbon (C) 3.4 g 100 g⁻¹ dry soil (chromium trioxide wet oxidation: colorimetric determination); nitrogen (N) 0.31 g 100 g⁻¹ dry soil (combustion elemental analyser: thermal conductivity detection); phosphorus (P) 10.0 μg mL 1 soil extraction (Olsen extraction: colorimetry) and potassium (K) 6 MAF units (QT=(μg mL⁻¹)/18.2) (ammonium acetate extraction: Flame Emission 15 Spectroscopy).

Mesocosms were stored in a cool area, watered regularly and any emerging weeds removed.

### Synchronization of Plant Material

The T₀ HME line (ODR6205) and wild type parent (Imp566) were propagated 3 rounds of synchronised clonal multiplication (in a controlled temperature room). During each round, five clones of five tillers each were potted in 1.3 L potting mix soil pots and grown for 4 weeks under 600-1000 μmol m⁻²s⁻¹ light, 12-h day-light, 20° C. day and 15° C. night temperature.

At the third round of clonal synchronising, all ramets were cut back after 4-5 weeks of propagation and spitted into 10 tillers/a bunch. 10 bunches were planted into pre-prepared divets in a soil mesocosm. For approximately eight weeks the mesocosms were watered daily and Phostrogen (NPK 16 10 24) (https://www.solabiol.com/en/phostrogen all-purpose plant food) applied at approximately bi-weekly intervals to support optimal plant growth. All mesocosms were weeded and their position on the bench rotated on a weekly basis to minimise any effect of position. Plants were cut to 60 mm above the soil surface at 3-4 weekly intervals as necessary. After approximately 8 weeks all plants were cut to 60 mm and ion exchange resins (Bowatte et al., 2008) were inserted into the centre of each mesocosm. The mesocosms were placed in designated row column position as per the experimental design and watered daily to a target volumetric water content (VWC) of 40% v/v. Soil moisture was monitored in five mesocosms using indwelling Time Domain Reflectometry (TDR) probes (ECH GS1 probes, FF Instrumentation, Christchurch, New Zealand) programmed to log at 10-minute intervals.

### N Treatments

Synthetic urine was made according to Clough et al. (1996) with urea being the main N source (91%) and hippuric acid the minor N source (9%) as recommended by Kool et al. (2006). The N content of the urine was adjusted by altering the amount of urea and hippuric acid added to the mixture with the ratio of urea and hippuric acid as a percentage of total N remaining the same (Table 1). The N treatments were applied at a volume equivalent to a standard dairy cow urine patch at 10 L m⁻² (Haynes & Williams, 1993) and the N concentration adjusted for each N rate. The synthetic urine was made up the day before application to the mesocosms and a sub sample of each of the N rates was analysed for total N content on the day of application. The constituents of the synthetic urine as well as the target N rates and the actual N rates are listed in Table 4.

TABLE 4

| | | Hippuric | | | | | |
|---|---|---|---|---|---|---|---|
| Target N rate | Urea | acid | KHCO₃ | KCl | KBr | K₂SO₄ | Actual N rate |
| (kg N ha⁻¹) | (g L⁻¹) | (g L⁻¹) | (g L⁻¹) | (g L⁻¹) | (g L⁻¹) | (g L⁻¹) | (kg N ha⁻¹) |
| 400 | 7.80 | 4.62 | 9.32 | 1.68 | 2.69 | 0.92 | 394 |
| 800 | 15.60 | 9.24 | 18.64 | 3.36 | 5.36 | 1.84 | 789 |

### N Treatment Application and Gas Sampling

Six days prior to N treatment the plants were cut to 60 mm above the soil level; four days before the N treatments were applied the N₂O emissions were measured; this was considered the base line for pre-N treatment application. The urine treatments were applied slowly and evenly to the mesocosm soil. Nitrous oxide emissions were measured 2 hours after urine application and then daily for 3 days, followed by thrice weekly sampling for a further 5 weeks. Thus, the schedule for measurements was-4 days; and at 2 hours and then at 1; 2; 3; 6; 8; 10 13; 15; 17; 20; 22; 24; 27; 30; 34; 37; and 41 days after urine application. This was followed by a gap until week 11 weeks (after urine application) and continued for an additional 3 weeks (days 78; 83; 90; 97). A static chamber technique was used to measure N₂O emissions. The gas sampling methodology is described in detail by van der Weerden et al. (2011). There were 23 gas sampling occasions in total over the 14-week period.

On each gas sampling day, N₂O measurements were carried out between 10:30 am and noon. On each occasion opaque PVC covers (190 mm diameter and 100 mm height) were placed on top of the mesocosms and secured with 100 mm wide rubber bands to provide an airtight seal. All covers had a rubber septum on the top surface to allow for gas sampling and six of the covers had an additional septum for monitoring chamber air temperature during the cover period. For all 36 mesocosms two headspace gas samples were taken at times 0 and 40 minutes during a 40 minute cover period. At each sampling six mesocosms at random were also sampled at 20 minutes to verify linearity of flux; 91% of the 40 minute cover times had an R2≥0.99 for linearity of flux, 95% an R2>0.98 and fewer than 1% an R2<0.90. In addition, the air temperature inside the gas cover was sampled at 0, 20 and 40 minutes for each of those 6 mesocosms. On each sampling day two background atmosphere samples were taken at the start and end of the 40 minute cover period and 50 mm soil temperatures were measured in six mesocosms at random immediately after each gas measurement.

Headspace gas samples were taken using a 25 mL polypropylene syringe and 20 mL was injected into a 12 mL septum sealed screw capped pre evacuated glass vial. Nitrous oxide analysis of the gas samples was conducted at Manaaki Whenua Landcare Research's Gas Analysis Laboratory using Shimadzu GC 17a and Shimadzu GC2010 gas chromatographs (Shimadzu Oceania Pty Ltd, Nelson, New Zealand); both were equipped with a 63Ni electron capture detector with oxygen free N as a carrier gas (Saggar et al., 2007).

Hourly N₂O emissions were calculated for each mesocosm from the linear increase in head space N₂O concentrations over the sampling time. Hourly N₂O emissions (mg N m⁻²h⁻¹) were calculated as follows (de Klein et al., 2003):

$${N}_{2} O flux = ( δ {N}_{2} O / δ t ) * ( M / Vm ) * ( V / A )$$

where N₂O flux is hourly N₂O emission (mg N₂O N m⁻²h⁻¹), ON₂O is the increase in headspace N₂O during the enclosure period (μL L⁻¹), δt is the enclosure period (h), M is the molar weight of N in N₂O (g mol 1), Vm is the molar volume of gas at the sampling temperature (L mol⁻¹), V is the headspace volume (m³), and A is the area covered (m²). Hourly emissions were integrated over time for each mesocosm using a trapezoidal integration to calculate the total emitted.

### Herbage Measurements

Herbage cuts were taken at approximately weekly to 2-weekly intervals over the gas measurement period. Plants were cut to 60 mm above the soil surface. The harvested herbage was immediately frozen in liquid N, stored in an 80° C. freezer until freeze drying for 24 h. After freeze drying samples were placed in a drying oven at 30° C. for 15-20 min and a dry weight taken.

Total Net Herbage Accumulation (g DM m⁻²)

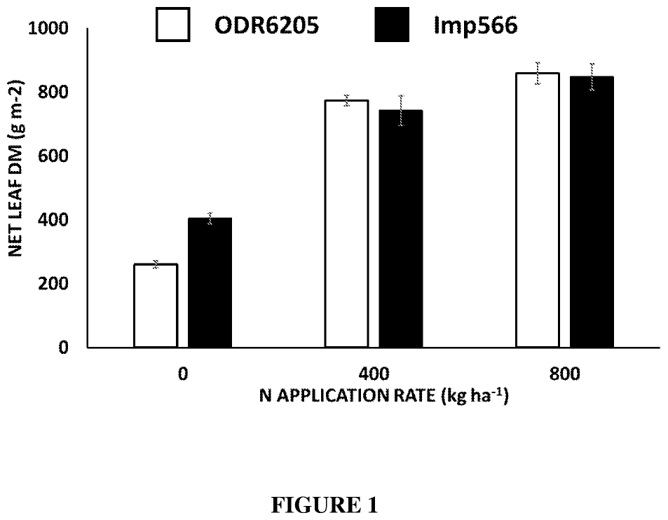

When no nitrogen was supplied to the plants total DM accumulation was greater for the Imp566 plants compared to the =ODR6205 plants. However, the Imp566 and ODR6205 plants accumulated similar amounts of DM to each other at both 400 and 800 N. There was a substantial increase in biomass comparing the zero N application with the 400N and a smaller increase comparing the biomass from the 400 N with the 800 N treatment (FIG. 1).

For both Imp566 and ODR6205 plants the DM accumulation was greater at 400 and 800 N than at 0 N.

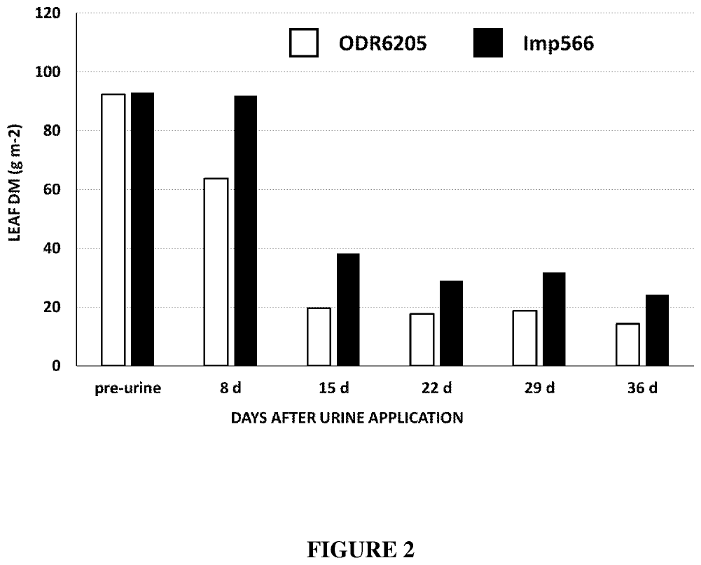

When no N was supplied the Imp566 plants accumulated more DM than the ODR6205 plants at each harvest interval (FIG. 2).

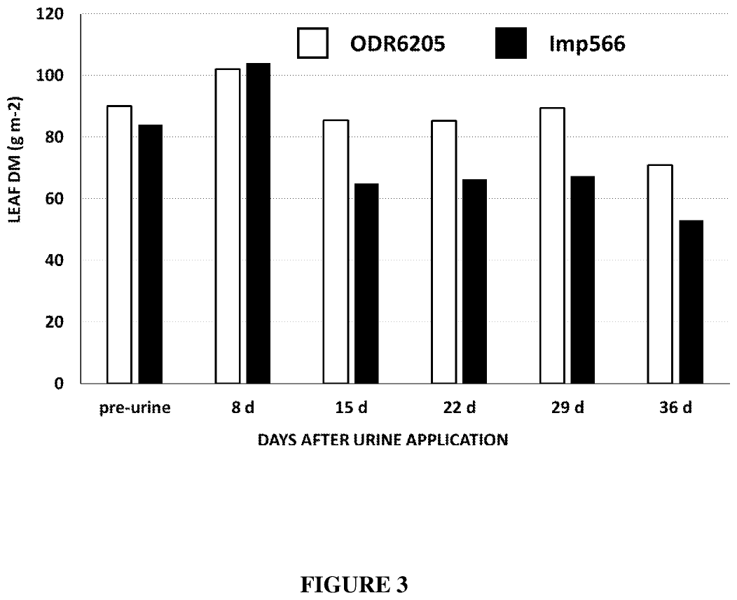

In the 400N treatment the ODR6205 plants line accumulated more DM than the Imp566 plants from days 15-36 (FIG. 3).

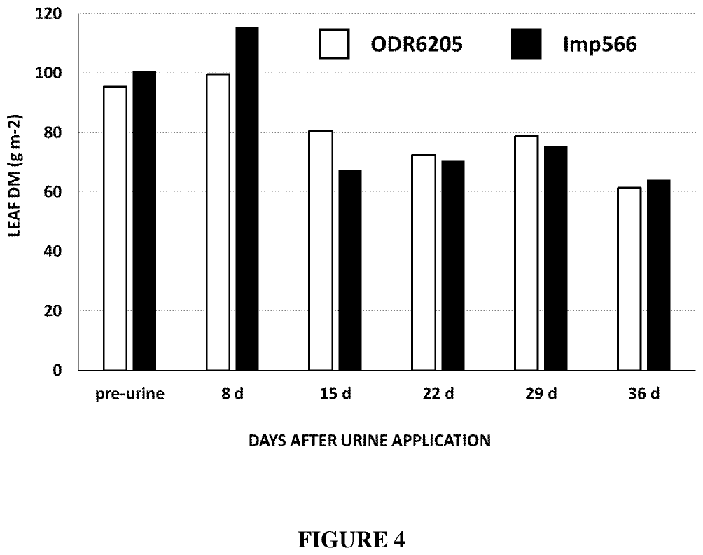

In the 800N treatment the ODR6205 plants line accumulated more DM than the Imp566 plants from days 15-29 (FIG. 4).

### Nitrous Oxide Emissions

N₂O flux over time (mg N₂O—N m⁻² hr⁻¹)

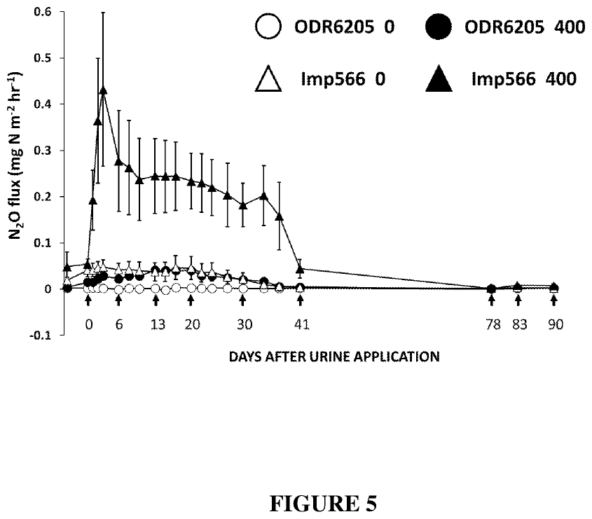

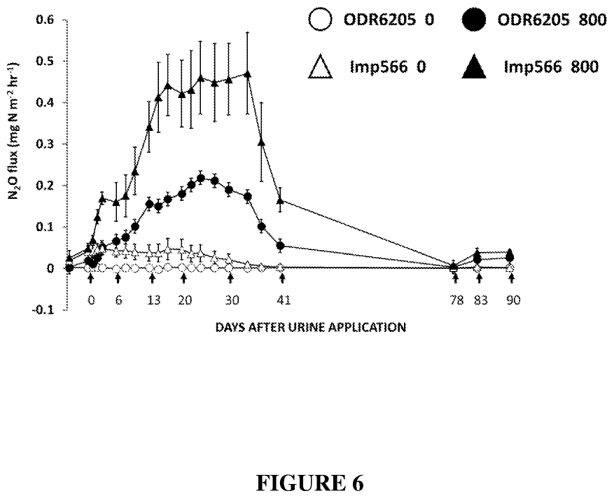

After the application of either 400 or 800 kg N ha⁻¹ equivalents to the mesocosms containing Imp566 plants there was a sharp increase in the N₂O flux; 30-35 days later this subsequently declined to levels approaching 0 N applications (FIG. 5, FIG. 6). A similar pattern was seen from the mesocosms containing ODR6205 plants after receiving 800 kg N ha⁻¹ equivalents; however, the peak amplitude of the increase was greatly reduced (FIG. 6). In contrast, the application of 400 kg N ha⁻¹ equivalents to the ODR6205 plants resulted in N₂O emissions comparable to 0 kg N/ha application to Imp566 plants, while ODR6205 plants that did not receive N (0 kg N ha 1 equivalents) had almost undetectable levels of N₂O emissions (FIGS. 5 and 6).

Cumulative N₂O—N emissions (mg N₂O—N m⁻²)

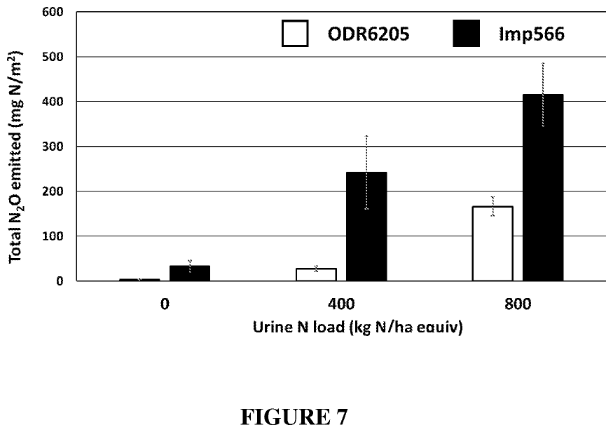

Increasing N application resulted in increased total N₂O emissions for Imp566 and ODR6205 plants; where the ODR6205 plants produced approximately 90.5, 88.6 and 60.1% less N₂O than WT at 0, 400 and 800 kg N/ha urine loads respectively. At the 0 and 400N applications rates the total emissions of N₂O nitrogen (N₂O—N) from mesocosms with ODR6205 plants were significantly less than from mesocosms containing Imp566 plants (FIG. 7). While the 800N application the N₂O—N from the mesocosms with Imp566 plantswas approximately 2.5 more than from the mesocosms with the ODR6205 plants; however, due to the variance this was not significantly greater (FIG. 7).

### Nitrous Oxide Emission Factor (EF)

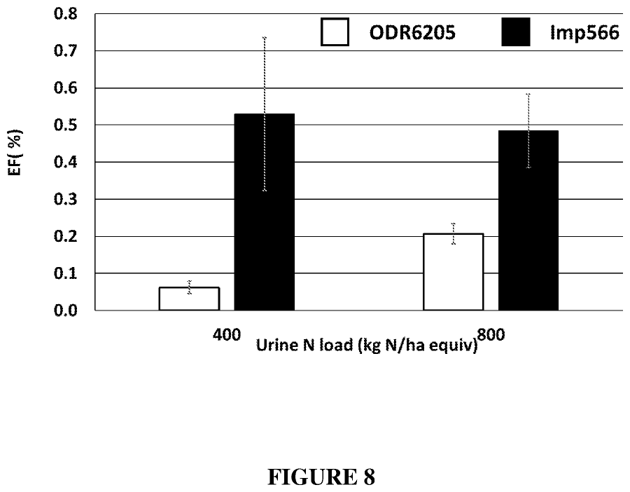

The EF for the HME was consistently lower than the Imp566 plantsand at the 400 kg ha⁻¹ N equivalents it was significantly lower (0.05 vs. 0.38%, respectively); whereas, at the high urine N rate the difference was large but it was not significant (0.21 and 0.44%, respectively) (FIG. 8).

### Example 7: Reduced N₂O Emissions from the Soil (after Application of Bovine Urine) in which ODR6205 and RCR5101 Plants Produced by Methods of the Invention (Biolistic and Agrobacterium Transformation Respectively) are Grown

The overall experimental design, set up and equipment was similar to that described in Example 6; the differences in Example 7 are described below.

### Treatments

Urine: A simulated bovine urine patch was created by using real bovine urine (collected 13 Sep. 2021 from Massey No 4-dairy farm); this was calibrated to provide a standard dairy cow urination event size of 10 L m⁻². The N load was 420 kg N ha⁻¹ (0.42 N %). Only a single N load was tested and a control (no urine) treatment was not included. The base line for zero application can however, be determined from the first two measurements which was prior to the application of urine.

### Plant Lines

1. T₀ ODR6205 vs IMP566 (from Example 2)
2. T₃ RCR5101 vs T₃ Null (from Example 3)

### Experimental Design:

The two comparisons were tested; each one in a separate control temperature room. The same mesocosm design as used in Example 6 were used. There was a total of 48 mesocosms with 24 for each comparison (each in a separate control temperature room). Destructive sampling was carried out at 0, 2, 4 weeks after urine application. At each sampling time, 4 replicates of each treatment were randomly selected for destructive plant, root and soil measurements. Consequently, the replicates for gas and resin N were reduced over time, i.e., 12 samples (Oct. 6-Oct. 15, 2021); 8 samples (Oct. 19-Nov. 1, 2021), 4 samples (Nov. 3-Nov. 15, 2021).

The PVC mesocosms were placed on 6 trolleys in the control temperature rooms. Trolleys were located under LED lighting (California Lightworks SolarSystem 550 Commercial Series) giving approximately 700 μmol m⁻²s⁻¹ light at the plant height. At the beginning 4 mesocosms per trolly. The position of the trolleys within the rooms were rotated clockwise and 180° every 2 days.

### Experimental Pots

The same mesocosm design as used in Example 6 but with an additional hole at 20 cm depth in order to insert a resin to measure N dynamics at depth; also, taller lids (18 cm) were used for gas measurements.

Soil: Soil for the mesocosms was collected from the AgResearch Grasslands campus, Palmerston North, New Zealand. The soil at the site was classified as a Karapoti silt loam (Dystric Eutrochrept). Soil properties at the time of collection 4 May 2021 (pH 5.6, Olsen Phosphorus 25 mg/L, Total C 2.3%, Total N 0.23%).

Gas: The same sampling protocol as described in Example 6 was used-emissions were measured at 2 hours after urine application and then daily for 3 days, followed by thrice weekly sampling for a further 3 weeks.

### Nitrous Oxide Emissions

N₂O Flux Over Time (Mg N₂O—N m⁻² hr⁻¹)

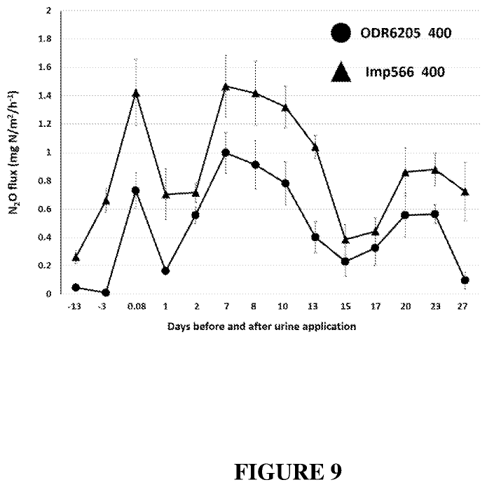

Prior to the application of the bovine urine the mesocosms containing the Imp566 plants had a higher emission of N₂O than those containing ODR6205 (FIG. 9). In the first 2 hours after urine application the mesocosms containing either Imp566 or ODR6205 plants showed substantial increases in N₂O flux followed by a smaller decrease over the next 24 hours. This was followed by a second rise and relatively sustained period of high N₂O flux and subsequent decline 10 days after urine application. In each measurement the level of N₂O flux from the mesocosms containing Imp566 was approximately 50% (or greater) higher than mesocosms containing ODR6205 (FIG. 9).

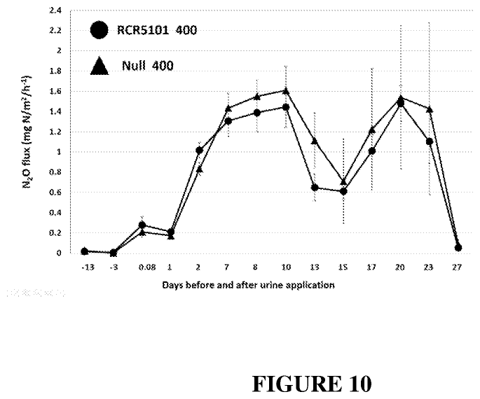

In comparison, prior to the application of the bovine urine, the mesocosms containing the homozygous RCR5101 and null plants had almost no N₂O flux; this was followed by increases up to approximately day 7. The levels dropped after day 10 followed by a subsequent rise to day 20 and then decreased to almost pre urine applications by day 27. From day 7 to day 23 the N₂O flux from the mesocosms containing null plants was typically 10% (or greater) than from mesocosms containing RCR5101 plants (FIG. 10).

### Reference Listing

Altpeter F., Xu J. and Ahmed S. 2000. Generation of largenumbers of independently transformed fertile perennial rye-grass (Lolium perenneL.) plants of forage- and turf typecultivars. Mol. Breeding 6:519-528.

Andrews M. A., Raven, J., & Lea, P. (2013). Do plants need nitrate? The mechanisms by which nitrogen form affects plants. Annals of Applied Biology, 163 (2), 174-199. York LM, Nord EA and Lynch JP (2013). Integration of root phenes for soil resource acquisition. Front Plant Sci. 2013 Sep. 12; 4:355. doi: 10.3389/fpls.2013.00355. eCollection 2013.

Bajaj, S., Ran, Y., Phillips, J., Kularajathevan, G., Pal, S., Cohen, D., Elborough, K., Puthigae, S. (2006). A high throughput Agrobacterium tumefaciens-mediated transformation method for functional genomics of perennial ryegrass (Lolium perenne L.). Plant Cell Reports 25, 651-659.

Bilang, R., Lida, S., Peterhans, A., Potrykus, I., Paszkowski, J., 1991. Gene, 100:247-250.

Bowatte S, Tillman R, Carran A, Gillingham A, Scotter D (2008) In situ ion exchange resin membrane (IEM) technique to measure soil mineral nitrogen dynamics in grazed pastures. Biology and Fertility of Soils, 44, 805-813.

Cassman, K. G., Dobermann, A., Wallers, D. T., Yang, H. S., 2003.Meeting cereal demand while protecting natural resources and improving environmental quality. Annu. Rev. Environ. Resour. 28, 315-358

Cayuela M L, van Zwieten L, Singh B P, Jeffery S. (2014). Biochar's role in mitigating soil nitrous oxide emissions: A review and meta-analysis. Agriculture, Ecosystems and Environment 191:5-16.

Chen, Y., Xiao, C., Chen, X., Li, Q., Zhang, J., Chen, F., Mi, G. (2014). Characterization of the plant traits contributed to high grain yield and high grain nitrogen concentration in maize. Field Crops Research, 159, 1-9.

Clough T J, Sherlock R R, Cameron K C, Ledgard S F (1996) Fate of urine nitrogen on mineral and peat soils in New Zealand. Plant and Soil, 178, 141-152.

de Klein C A M, Barton L, Sherlock R R, Li Z, Littlejohn R P (2003). Estimating a nitrous oxide emission factor for animal urine from some New Zealand pastoral soils. Australian Journal of Soil Research 41 (3) 381-399.

Dijkstra, F A, Zhu B, Cheng W (2020). Root effects on soil organic carbon: a double-edged sword. New Phytologist 230:60-65

Feeney, M., Punja, Z. K. Tissue culture and Agrobacterium-mediated transformation of hemp (Cannabis sativa L.). In Vitro Cell Dev Biol-Plant 39, 578-585 (2003).

Frandsen et al., (2001). Physiologia Plantarum, 112:301-307.

Garnett, T., Conn, V., Kaiser, B. N., 2009. Root based approaches to improving nitrogen use efficiency in plants. Plant Cell Environ. 32, 1272-1283.

Guo, J. H., Liu, X. J., Zhang, Y., Shen, J. L., Han, W. X., Zhang, W. F., Christie, P., Goulding, K. W. T., Vitousek, P. M., Zhang, F. S., 2010. Significant acidification in major Chinese croplands. Science 327, 1008-1010

Haynes R J, Williams P H (1993) Nutrient cycling and soil fertility in the grazed pasture ecosystem. In: Advances in Agronomy. (ed Donald LS) pp Page., Academic Press.

Huang (1992). Ann. Rev. Plant Physiol. Plant Mol. Biol. 43:177-200.

Ju, C., Buresh, R. J., Wang, Z., Zhang, H., Liu, L., Yang, J., & Zhang, J. (2015). Root and shoot traits for rice varieties with higher grain yield and higher nitrogen use efficiency at lower nitrogen rates application. Field Crops Research, 175, 47-55.

Kool D M, Hoffland E, Abrahamse S, Van Groenigen J W (2006) What artificial urine composition is adequate for simulating soil N₂O fluxes and mineral N dynamics? Soil Biology and Biochemistry, 38, 1757-1763.

Kyte and Doolitle (1982) J. Mol. Biol. 157:105-132

Leprince et al., (1998). Planta 204 109-119.

Lin and Tzen. (2004). Plant Physiology and Biochemistry. 42:601-608.

Loer and Herman (1993). Plant Physiol. 101 (3): 993-998.

Murphy (1993). Prog. Lipid Res. 32:247-280.

Ohlrogge and Jaworski (1997). Annu Rev Plant Physiol Plant Mol Biol. 48:109-136.

Pang, J., Milroy, S. P., Rebetzke, G. J., & Palta, J. A. (2015). The influence of shoot and root size on nitrogen uptake in wheat is affected by nitrate affinity in the roots during early growth. Functional Plant Biology, 42 (12), 1179-1189.

Peng, S., Tang, Q., Zou, Y., (2009). Current status and challenges of rice production in China. Plant Prod. Sci. 12, 3-8.

Rasse D P, Rumpel C, Dignac M-F (2005). Is soil carbon mostly root carbon? Mechanisms for a specific stabilisation. Plant and Soil 269:341-256.

Raun, W. R., Johnson, G. V., 1999. Improving nitrogen use efficiency for cereal production. Agron. J. 91, 357-363.

Roberts et al., (2008). The Open Biotechnology Journal 2:13-21.

Saggar S, Hedley C B, Giltrap D L, Lambie S M (2007) Measured and modelled estimates of nitrous oxide emission and methane consumption from a sheep-grazed pasture. Agriculture, Ecosystems and Environment, 122, 357-365.

Sarmiento et al., (1997). Plant J. 11 (4): 783-96.

Shockey et al., (2006). Plant Cell., 18,2294-2313.

Signor D and Cerri CEP (2013). Nitrous oxide emissions in agricultural soils: a review. Pesq. Agropec. Trop., Goiânia, v. 43, n. 3, p. 322-338

Siloto et al., (2006). Plant Cell. 18 (8): 1961-74.

Shimada et al., (2008). Plant J. 55 (5): 798-809.

Slack et al., (1980). Biochem J. 190 (3): 551-561.

Tzen et al., (1997). J Biochem. 121 (4): 762-8.

Tzen et al., (1992). J. Biol. Chem. 267:15626-34

Tzen et al., (2003). Adv Plant Physiol., 6:93-104.

Uchida Y, and von Rein I (2018). Mitigation of nitrous oxide emissions during nitrification and denitrification processes in agricultural soils using enhanced efficiency fertilizers. IntechOpen DOI: http://dx.doi.org/10.5772/intechopen.81548

van Der Weerden T J, Luo J, De Klein CaM, Hoogendoorn C J, Littlejohn R P, Rys G J (2011) Disaggregating nitrous oxide emission factors for ruminant urine and dung deposited onto pastoral soils. Agriculture, Ecosystems & Environment, 141, 426-436.

Velthof G L and Rietra R P J J (2018). Nitrous oxide emission from agricultural soils. Wageningen Environmental Research Report 2921, ISSN 1566-7197. Wageningen University and Research.

Vitousek, P. M., Mooney, H. A., Lubchenco, J., Melillo, J. M., 1997. Human domination of Earth's ecosystems. Science 277, 494-499.

Winichayakul, S., Scott, R. W., Roldan, M., Hatier, J.-H. B., Livingston, S., Cookson, R., Curran, A. C., Roberts, N.J. (2013). In vivo packaging of triacylglycerols enhances Arabidopsis leaf biomass and energy density. Plant Physiology 162, 626-639.

Xu J, Francis T, Mietkiewska E, Giblin E M, Barton D L, Zhang Y, et al. Cloning and characterization of an acyl-CoA-dependent diacylglycerol acyltransferase 1 (DGAT1) gene from Tropaeolum majus, and a study of the functional motifs of the DGAT protein using site-directed mutagenesis to modify enzyme activity and oil content. Plant Biotechnol J. 2008; 6:799-818.

York L M, Nord E A, Lynch J P (2013). Integration of root phenes for soil resource acquisition. Front Plant Sci. 2013 Sep. 12; 4:355. doi: 10.3389/fpls.2013.00355. eCollection 2013. PMID: 24062755

SUMMARY OF SEQUENCE LISTING

| SEQ ID | | | |
|---|---|---|---|
| NO: | Type | SPECIES | COMMENTS |
| 1 | Polypeptide | S. indicum | AAG23840 |
| 2 | Polypeptide | S. indicum | AAB58402 |
| 3 | Polypeptide | A. thaliana | CAA44225 |
| 4 | Polypeptide | A. thaliana | AAZ23930 |
| 5 | Polypeptide | H. annuus | CAA44224.1 |
| 6 | Polypeptide | B. napus | CAA57545.1 |
| 7 | Polypeptide | Z. mays | NP_001147032.1 |
| 8 | Polypeptide | O. sativa | AAL40177.1 |
| 9 | Polypeptide | B. oleracea | AAD24547.1 |
| 10 | Polypeptide | C. arabica | AAY14574.1 |
| 11 | Polypeptide | B. oleraceae | CAA65272.1 |
| 12 | Polypeptide | S. indicum | AAD42942 |
| 13 | Polynucleotide | S. indicum | AF302907 |
| 14 | Polynucleotide | S. indicum | U97700 |
| 15 | Polynucleotide | A. thaliana | X62353 |
| 16 | Polynucleotide | A. thaliana | BT023738 |
| 17 | Polynucleotide | H. annuus | X62352.1 |
| 18 | Polynucleotide | B. napus | X82020.1 |
| 19 | Polynucleotide | Z. mays | NM_001153560.1 |
| 20 | Polynucleotide | O. sativa | AAL40177.1 |
| 21 | Polynucleotide | B. oleracea | AF117126.1 |
| 22 | Polynucleotide | C. arabica | AY928084.1 |
| 23 | Polynucleotide | B. oleraceae | X96409 |
| 24 | Polynucleotide | S. indicum | AF091840 |
| 25 | Polypeptide | A. thaliana | NP_179535 |
| 26 | Polypeptide | T. majus | AAM03340 |
| 27 | Polypeptide | Z. mays | ABV91586 |
| 28 | Polypeptide | A. thaliana | NP_566952 |
| 29 | Polypeptide | B. napus | AC090187 |
| 30 | Polypeptide | A. hypogaea | AAX62735 |
| 31 | Polypeptide | A. thaliana | NP_196868 |
| 32 | Polypeptide | R. communis | XP_002521350 |
| 33 | Polynucleotide | A. thaliana | NM_127503 |
| 34 | Polynucleotide | T. majus | AY084052 |
| 35 | Polynucleotide | Z. mays | EU039830 |
| 36 | Polynucleotide | A. thaliana | NM_115011 |
| 37 | Polynucleotide | B. napus | FJ858270 |
| 38 | Polynucleotide | A. hypogaea | AY875644 |
| 39 | Polynucleotide | A. thaliana | NM_121367 |
| 40 | Polynucleotide | R. communis | XM_002521304 |
| 41 | Polynucleotide | O. sativa | AY583764 |
| 42 | Polynucleotide | O. sativa | AP014965 |
| 43 | Polynucleotide | P. vulgaris | AF028707 |
| 44 | Polynucleotide | P. sativum | M21356; M27973 |
| 45 | Polynucleotide | P. sativum | M64619 |
| 46 | Polynucleotide | G. max | D16248 |
| 47 | Polynucleotide | A. thaliana | L05399 |
| 48 | Polynucleotide | Cauliflower | V00141; J02048 |
| | | mosaic virus | |
| 49 | Polypeptide | S. indicum | Modified oleosin |
| 50 | Polypeptide | T. majus | DGAT1 |
| 51 | Polypeptide | G. max | Oleosin |
| 52 | Polynucleotide | G. max | Oleosin |
| 53 | Polypeptide | G. max | Oleosin |
| 54 | Polynucleotide | G. max | Oleosin |
| 55 | Polypeptide | G. max | Oleosin |
| 56 | Polynucleotide | G. max | Oleosin |
| 57 | Polypeptide | G. max | Oleosin |
| 58 | Polynucleotide | G. max | Oleosin |
| 59 | Polypeptide | Z. mays | Oleosin |
| 60 | Polynucleotide | Z. mays | Oleosin |
| 61 | Polypeptide | G. max | DGAT1 |
| 62 | Polynucleotide | G. max | DGAT1 |

1. A method for producing a plant that reduces N₂O production from the soil in which it is grown, the method comprising the step of genetically modifying the plant to express a modified oleosin including at least one artificially introduced cysteine.

2. The method of claim 1 comprising the additional step of assessing the capability of the plant produced to reduce N₂O production from the soil in which it is grown, and selecting the plant based on this assessment.

3. A method for reducing N₂O production in soil, the method comprising growing in the soil at least one of:
a) a plant produced by the method of claim 1, and
b) a plant genetically modifying the plant to express a modified oleosin including at least one artificially introduced cysteine
in the soil.

4. The method of claim 1 in which the plant is also genetically modified to express at least one triacylglycerol (TAG) synthesizing enzyme.

5. The method of claim 1 in which the reduction in N₂O production from the soil in which the plant is grown is relative to N₂O production from the same soil in the absence of the plant.

6. The method of claim 1 in which the reduction in N₂O production from the soil in which the plant is grown is relative to N₂O production in the same soil from which a control plant is grown.

7. The method of claim 1 in which the reduction in N₂O production is assessed from soil in which multiple plants are grown relative to that in the same soil in which the same number of control plants are grown.

8. The method of claim 1 in which N₂O production is reduced by at least 5%.

9. The method of claim 1 in which N₂O production is reduced over a period of at least 1 day.

10. The method of claim 1 in which the soil has a Nitrogen (N) load of at least 10 kg N/ha

11. The method of claim 1 including the step of production of seed from the plant, or plants produced.

12. The method of claim 11 in which the seed is promoted, marketed or labelled, as any one of:
a) reducing green house gas (GHG) production in soil in which the seeds, and/or resulting plants, are grown,
b) reducing N₂O production in soil in which the seeds, and/or resulting plants, are grown,
c) having environmental, social, and governance (ESG) benefits as a result of a) or b).

13. A plant or seed produced by the method of claim 11 that is promoted or marketed as any one of:
a) reducing green house gas (GHG) production in soil in which the plant or seed is grown,
b) reducing N₂O production in soil in which the plant or seed is grown,
c) having environmental, social, and governance (ESG) benefits as a result of a) or b).

14. A package containing seed produced by the method of claim 11 promoted, marketed or labelled, as any one of:
a) reducing green house gas (GHG) production from soil in which the seeds, and/or resulting plants, are grown,
b) reducing N₂O production from soil in which the seeds, and/or resulting plants, are grown,
c) having environmental, social, and governance (ESG) benefits as a result of a) or b).

15. A plant produced by the method of claim 1 for use in reducing N₂O production from the soil in which it is grown, or when used to reduce N₂O production from the soil in which it is grown.

16. A plant produced by the method of claim 11 for use in reducing N₂O production from the soil in which it is grown, or when used to reduce N₂O production from the soil in which it is grown.


SEQUENCE LISTING (62 sequences)

SEQ ID NO: 1 - protein, 166 aa, Sesamum indicum
MADRDRPHPHQIQVHPQHPHRYEGGVKSLLPQKGPSTTQILAIITLLPISGTLLCLAGITLVGTLIGLAVATPVFVIFSPVLVPAAILIAGAVTAFLTSGAFGLTGLSSLSWVLNSFRRATGQGPLEYAKRGVQEGTLYVGEKTKQAGEAIKSTAKEGGREGTART

SEQ ID NO: 2 - protein, 144 aa, Sesamum indicum
MADEPHDQRPTDVIKSYLPEKGPSTSQVLAVVTLFPLGAVLLCLAGLILTGTIIGLAVATPLFVIFSPILVPAALTIALAVTGFLTSGAFGITALSSISWLLNYVRRMRGSLPEQLDHARRRVQETVGQKTREAGQRSQDVIRP

SEQ ID NO: 3 - protein, 173 aa, Arabidopsis thaliana
MADTARGTHHDIIGRDQYPMMGRDRDQYQMSGRGSDYSKSRQIAKAATAVTAGGSLLVLSSLTLVGTVIALTVATPLLVIFSPILVPALITVALLITGFLSSGGFGIAAITVFSWIYKYATGEHPQGSDKLDSARMKLGSKAQDLKDRAQYYGQQHTGGEHDRDRTRGGQHTT

SEQ ID NO: 4 - protein, 149 aa, Arabidopsis thaliana
MADHQQHQQQQQPIMRSLHESSPSTRQIVRFVTAATIGLSLLVLSGLTLTGTVIGLIVATPLMVLFSPVLVPAVITIGLLTMGFLFSGGCGVAAATALTWIYKYVTGKHPMGADKVDYARMRIAEKAKELGHYTHSQPQQTHQTTTTTH

SEQ ID NO: 5 - protein, 181 aa, Helianthus annuus
TTTTYDRHFTTTQPHYRQDDRSRYDQQTHSQSTSRTLAIIALLPVGGILLGLAALTFIGTLIGLALATPLFVIFSPIIVPAVLTIGLAVTGFLASGTFGLTGLSSLSYLFNMVRQTAGSVPESLDYVKGTLQDAGEYAGQKTKDFGQKIQSTAHEMGDQGQVGVHAQVGGGKEGRKSGDRT

SEQ ID NO: 6 - protein, 165 aa, Brassica napus
MGILRKKKHERKPSFKSVLTAILATHAATFLLLIAGVSLAGTAAAFIATMPLFVVFSPILVPAGITTGLLTTGLAAAGGAGATAVTIILWLYKRATGKAPPKVLEKVLKKIIPGAAAAPAAAPGAAPAAAPAAAPAVAPAAAPAAAPAPKPAAPPAPKPAAAPSI

SEQ ID NO: 7 - protein, 186 aa, Zea mays
MADRDRSGIYGGAHATYGQQQQQGGGGRPMGEQVKGMLHDKGPTASQALTVATLFPLGGLLLVLSGLALTASVVGLAVATPVFLIFSPVLVPAALLIGTAVMGFLTSGALGLGGLSSLTCLANTARQAFQRTPDYVEEAHRRMAEAAAHAGHKTAQAGQAIQGRAQEAGAGGGAGAGAGGGGRASS

SEQ ID NO: 8 - protein, 148 aa, Oryza sativa
MGDQHRGVIGGGGYGDRGGQEQQEKQPFMMTALKTVTAATAGGSILVLSGLILAGTVIALTVATPVLVIFSPVLVPAAIALALMAAGFVTSVGLGVAALSVFSWMYKYLTGKHPPGADHLDHTKARVASKLRDIKEAAHHLIDQAQAS

SEQ ID NO: 9 - protein, 216 aa, Brassica oleracea
RFFRMFSFIFPLLNVIKLIIASVTSLVCLAFSCVTLGGSAVALIVSTPLFIIFSPILVPATIATTLLASGLMAGTTLGLTGIGLITGLVRTAGGVTLAESPIRRIIINRIKARLGGGGGSRLAMLKKILGLIKKLRGMSSGGAAPALKQHQQLRPRMELHPRHLHRPNKERWFMLFQYVAHKNCVIINLRIYDSETKKKIALLLSFIQYSFLCNNV

SEQ ID NO: 10 - protein, 148 aa, Coffea arabica
MAEHYQLQQRPTEAVKSFLPQKGPSTSHVLAVVTLLPVAGVLLGLSGLILVGTVIGLAVTTPLFVIFSPILVPAVFALGLALAGFLTSGAFGITALASLSWMLNYIRLMKASSQEQMDLAKWRVQDTAGQVGQKARDVGQRTQDVARA

SEQ ID NO: 11 - protein, 380 aa, Brassica oleracea
MRNEIQNETAQTDQTQGSMFSFFDLFPFLLPMFEVIKMVVASVASVVYLGFAGVTLSGSAVALAVSTPLFIIFSPILLPAIAATTVLAAGLGSKKVAAAPAASPSLSLLGIPESIKPSNVIPESIKPSNIIPESIKPSNIIPESVKPSNIKDKIKDTIGKVKNKINAKKEEKSKGKSEDSSKGKGKSKGEDTTTDEDKPGSGGKHGKGESKHGKGESTHGKGGKHGSEGSSMDEGKHGGKHGSGGSPMGVGKHGSGGKHESGGSPMGGGKHGSGGKHESGGASMGGGKHGSGGRHEGGGSAMGGGKHGSGGKHGSEGKHGGEGSSMGKNSLSKNKKEFHYRDQAMDASSTSESSDGSSDGSSSDGSSSDGSSHGSGGKHI

SEQ ID NO: 12 - protein, 145 aa, Sesamum indicum
MAEHYGQQQQTRAPHLQLQPRAQRVVKAATAVTAGGSLLVLSGLTLAGTVIALTIATPLLVIFSPVLVPAVITIFLLGAGFLASGGFGVAALSVLSWIYRYLTGKHPPGADQLESAKTKLASKAREMKDRAEQFSQQPVAGSQTS

SEQ ID NO: 13 - DNA, 501 bp, Sesamum indicum
  source 1..501: mol_type = other DNA
atggcggaccgcgaccgtccacacccccaccaaattcaagtccaccctcaacatccgcaccgctatgaaggtggcgtcaagtctctcctccctcaaaagggcccctccaccacccagattctcgccataatcaccctccttcccatcagcggcacgcttctttgcctagctgggatcacgctcgtcgggaccctcatcggacttgcagtcgccaccccagtcttcgtgatcttcagccctgttctggttcccgcagccatactgatagccggcgcggtcacggcgtttttgacgtccggggcttttgggctgacggggctttcgtcgctttcttgggttctgaattcattcagacgggcgacggggcaggggccgttggagtacgcgaagcgaggcgtgcaggaggggactttgtatgtgggagagaagacgaagcaagcgggcgaagcgattaagagcacagccaaggaaggagggcgagaagggactgcacggacttga

SEQ ID NO: 14 - DNA, 559 bp, Sesamum indicum
  source 1..559: mol_type = other DNA
ggcacgagcgccgtccccatggcggacgaaccccacgaccagcgccccaccgacgtcatcaagagctacctccccgaaaagggtccctccacctctcaagtcctcgccgtcgtgaccctcttccccctcggcgccgtcctcctctgcctagccggtctcattcttaccgggaccatcatcggcctcgccgtcgccaccccgctcttcgtcatcttcagccccatcttggtccccgccgccctaaccatcgccctagccgtcaccggtttcttgacctccggagctttcggcatcaccgccctgtcctcgatttcgtggttgctgaactacgttaggcgaatgcgggggagcttgccagagcagctggatcatgcacggcggcgcgtgcaggagacggtgggccagaagacaagggaggcggggcagagaagccaagatgtaataagaccgtgaggtttttggatattagatgttggttaatttgtgtgtttaatgtatatatgaggggttgaataagttaataaaattgcggatttggtacaaaaaaaaaaaaaaaaaa

SEQ ID NO: 15 - DNA, 1800 bp, Arabidopsis thaliana
  source 1..1800: mol_type = other DNA
ccatggctatacccaacctcggtcttggtcacaccaggaactctctggtaagctagctccactccccagaaacaaccggcgccaaattgccggaattgctgacctgaagacggaacatcatcgtcgggtccttgggcgattgcggcggaagatgggtcagcttgggcttgaggacgagacccgaatcgagtctgttgaaaggttgttcattgggatttgtatacggagattggtcgtcgagaggtttgagggaaaggacaaatgggtttggctctggagaaagagagtgcggctttagagagagaattgagaggtttagagagagatgcggcggcgatgacgggaggagagacgacgaggacctgcattatcaaagcagtgacgtggtgaaatttggaacttttaagaggcagatagatttattatttgtatccattttcttcattgttctagaatgtcgcggaacaaattttaaaactaaatcctaaatttttctaattttgttgccaatagtggatatgtgggccgtatagaaggaatctattgaaggcccaaacccatactgacgagcccaaaggttcgttttgcgttttatgtttcggttcgatgccaacgccacattctgagctaggcaaaaaacaaacgtgtctttgaatagactcctctcgttaacacatgcagcggctgcatggtgacgccattaacacgtggcctacaattgcatgatgtctccattgacacgtgacttctcgtctcctttcttaatatatctaacaaacactcctacctcttccaaaatatatacacatctttttgatcaatctctcattcaaaatctcattctctctagtaaacaagaacaaaaaaatggcggatacagctagaggaacccatcacgatatcatcggcagagaccagtacccgatgatgggccgagaccgagaccagtaccagatgtccggacgaggatctgactactccaagtctaggcagattgctaaagctgcaactgctgtcacagctggtggttccctccttgttctctccagccttacccttgttggaactgtcatagctttgactgttgcaacacctctgctcgttatcttcagcccaatccttgtcccggctctcatcacagttgcactcctcatcaccggttttctttcctctggagggtttggcattgccgctataaccgttttctcttggatttacaagtaagcacacatttatcatcttacttcataattttgtgcaatatgtgcatgcatgtgttgagccagtagctttggatcaatttttttggtcgaataacaaatgtaacaataagaaattgcaaattctagggaacatttggttaactaaatacgaaatttgacctagctagcttgaatgtgtctgtgtatatcatctatataggtaaaatgcttggtatgatacctattgattgtgaataggtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggccagcacactacttaagttaccccactgatgtcatcgtcatagtccaataactccaatgtcggggagttagtttatgaggaataaagtgtttagaatttgatcagggggagataataaaagccgagtttgaatctttttgttataagtaatgtttatgtgtgtttctatatgttgtcaaatggtacc

SEQ ID NO: 16 - DNA, 450 bp, Arabidopsis thaliana
  source 1..450: mol_type = other DNA
atggcggatcatcaacaacatcagcaacaacaacaaccaataatgaggagtctccatgaatcatcaccatcgactcggcagatagtgagattcgtaacggcagctacgatcggcctatcactcctcgtgctctcaggactaacactaaccggaacggtgatcggtttgatcgtagcgacgccgttgatggttctgttcagcccggtgttggtaccggcagtgataacgatagggcttctgacgatgggattcctattctccggtggttgtggggtggcagcagctacggcgttaacgtggatttataagtacgttaccggaaaacacccgatgggagcggataaggtggattacgcgaggatgaggatagcggagaaagccaaagagttgggacattatacgcactcgcagccacaacaaacacaccaaaccacaacaactactcattag

SEQ ID NO: 17 - DNA, 625 bp, Helianthus annuus
  source 1..625: mol_type = other DNA
actaccaccacttacgaccgtcatttcaccaccacccaaccccactaccgccaagacgacaggtcccgctacgaccagcaaacccattcccagtccaccagcaggacactcgccatcatcgccctacttcctgtcggcggaatcttactcggcttagccgctctcacattcatcgggacgcttatcgggctcgccctcgccaccccgcttttcgtcatattcagcccgatcatcgtgccggccgttctaacaatcgggcttgctgttacaggctttttggcgtcggggacgttcgggttgacgggtttgagctcattgtcgtatttgttcaatatggttaggcagacggctgggtcggtgcccgagtccttggattatgttaaggggacgttgcaggatgccggtgagtatgccgggcagaagacgaaggatttcgggcagaagattcagagcacggctcatgagatgggtgatcaggggcaggttggtgttcatgctcaagttggtggcgggaaagaagggcgaaaaagcggtgatcggacttgaggattcaaggttgatattgtggaataataatgttgatgtaagtttttagtgttatcaaagctttgtttgtttgtttgta

SEQ ID NO: 18 - DNA, 737 bp, Brassica napus
  source 1..737: mol_type = other DNA
gaattccattcaaactagtaaacaatggggatactcaggaagaaaaaacacgagcgaaagccgtcgtttaagagtgttttaaccgcaatattagctacacatgccgcaacattcctcttgttgatcgccggtgtatccctcgccggcacagccgccgcatttatcgctaccatgccactattcgtagtattcagtccgattctcgtaccagctggtattaccactggtttactgactacgggtttagcagccgccggtggcgccggcgcgactgctgtcaccatcatcctgtggctctacaagcgagcaacgggcaaggcgccgccaaaagtcctagaaaaagtcttgaaaaagataataccaggtgctgcagctgcaccagcagccgctccaggagccgctccagcagcggcgccagcagccgcaccagctgtggcgccagcagccgcaccagctgctgcgccagcacctaagccagcagccccaccagcacctaagccagcagccgcaccgagtatatgaaaagaagtggtgggcatgagtaaaggttgatatggaaaactggatacatagaaaaaagagtaatccaacttttaaaaaataaataacaacttcacgtggggatagaaaaattttcaaatattattttactaatggatgtcgcggtacaaaataataacaaatgtaagcctttttattgtatagtattttaagaacgaagctatgtagcgttgaca

SEQ ID NO: 19 - DNA, 1153 bp, Zea mays
  source 1..1153: mol_type = other DNA
aactactccgtcactttgtttgcaaagctcctcctcgatccatcgatcactgcaccggccggcggcaccgcgctcgcaggggctagccaacgagacggcagcaatggcggaccgtgaccgcagcggcatctacggcggcgcccacgccacctacgggcagcagcagcagcagggaggaggcgggcgcccgatgggtgagcaggtgaagggcatgctccacgacaaggggccgacggcgtcgcaggcgctgacggtggcgacgctgttcccgctgggcgggctgctgctggtgctgtcggggctggcgctgacggcctccgtggtggggctggccgtggccacgccggtgttcctgatcttcagccccgtgctggtccccgccgcgctgctcatcgggacggccgtcatggggttcctcacgtcgggcgcgctggggctcgggggcctgtcctcgctcacgtgcctcgccaacacggcgcggcaggcgttccagcgcaccccggactacgtggaggaggcgcaccgcaggatggcggaggccgcggcgcacgcgggccacaagaccgcgcaggcaggccaggccatccagggcagggcgcaggaggccggcgccgggggaggtgcaggtgccggcgctggcggcggcggcagggcttcctcgtaagcaagtcatccatgcatggattatggatagatgcgcgcgtgcgtgtctatcagtatcagcagccagcagggtcgtcgcggaatgctgtgttcctgtacgtgtgggtgaccgtccttccgtccttcgtctttctccccccgagtgtgtgttacgtatgtcctggtgttcgtcgtgtgtgttcatcgccgctccagttgaattccggtgtctgttcatcgccgctccaggtcgtagatgtgaatatactttgctaggggaataagtgataagtctgtctggaaggtaatgtttgagctttgctagtgtggctgggcactctggtcactggttgtgttgtgcatgcatcagctgtatgatcgtcgtctgttgtggaaaattggtcaatgtattctcttgctgaataatttgtgacatctaattgttatgtatcgtctctttgctgaataatcagtttctgatttatcttgcattaaaaaaaaaaaaaaaaaa

SEQ ID NO: 20 - DNA, 447 bp, Oryza sativa
  source 1..447: mol_type = other DNA
atgggtgatcagcacagaggagtgatcggcggtggcggctacggtgaccgtggtggccaggagcagcaggagaagcagcccttcatgatgacggctctgaagacggtgaccgcggcgacggccgggggctcgattctggtgctgtccgggctgatcctggccgggaccgtcatcgcgctcacggtggccaccccggtgttggtcatcttcagccccgtgctggtaccggcggccatcgcgctggcgctcatggcggccgggttcgtcacctcggttggtctcggtgtagccgcgctctccgttttctcgtggatgtacaagtacctcaccgggaagcacccgccgggcgccgaccatctggaccacaccaaggcgagggtcgcgtccaagctccgcgacatcaaggaggcggcgcatcacctcatcgaccaggcgcaggcgtcttag

SEQ ID NO: 21 - DNA, 709 bp, Brassica oleracea
  source 1..709: mol_type = other DNA
gccgttttttcagaatgttctcttttatcttcccattgctgaacgttataaagcttattatagcttccgtgacctccttagtctgcttagcgttttcttgtgtgacactcggtggttcagccgtggcattaatcgtatccacaccacttttcatcatatttagtccaattctcgtacctgccactattgccactaccctcctagccagtgggctcatggcgggtaccaccctcggactgaccggcataggtctcatcacggggctcgttaggacggcaggaggagttacattggccgaatcaccgataagaagaattataataaatagaattaaagcaagacttgggggtggcggcggttcacgtctggcaatgctcaaaaaaattctgggactcattaaaaagttgcgtggtatgtcttcaggtggagcagcacctgcgctgaagcagcaccagcagctgcgcccgcggatggagctgcacccgcggcacctgcaccgacctaacaaagaacgttggttcatgctgttccaatatgtagcacataaaaattgtgtaataattaacttaagaatttatgattcggaaactaaaaagaaaatagcccttttactatcttttatacaatatagttttctatgtaataatgtttaatttgcttataactataaaagactcatgcatagttgattaggaaaaaaaaaaaaaaa

SEQ ID NO: 22 - DNA, 447 bp, Coffea arabica
  source 1..447: mol_type = other DNA
atggctgagcactaccagctgcagcaacgccccacagaggccgtcaaaagcttccttcctcagaagggtccatcaacttcacatgtgttagcagttgtcacgctcctcccagttgcgggagtcctgctaggcctttccgggctgattctcgtcggaacggtcatcggtctggcggtgacaaccccgcttttcgttatctttagccccattttggtcccagctgtatttgccctagggctggccctggccgggttcttgacctccggtgctttcgggatcactgcacttgcttcattgtcgtggatgctgaactacatccgactcatgaaggcgtcttcccaggagcaaatggacctcgcaaagtggcgcgtgcaggacactgccggccaagttggtcagaaagcgagagacgtgggccagagaactcaagatgtagccagagcatga

SEQ ID NO: 23 - DNA, 1000 bp, Brassica oleracea
  source 1..1000: mol_type = other DNA
atgagaaacgaaattcaaaacgaaacagctcagactgatcagacacagggaagtatgttttcttttttcgatttgttccctttcctcctcccaatgtttgaggttatcaagatggttgttgcttccgttgcgtccgtagtctatttaggcttcgccggtgtaacactcagtggttcagccgtggcattagccgtatctacccctcttttcatcatattcagtccgattctcttacctgctattgcagccactactgtcctagccgcagggctcggcagtaaaaaagttgcggcggctccggcagcttcaccatccctatctctgttgggcataccggagagtattaaaccaagtaatgttataccggagagtattaaaccaagtaatattataccggagagtattaaaccaagtaatattataccggagagtgttaaaccaagtaatattaaggacaaaattaaggatacgataggcaaagttaagaataagatcaatgctaaaaaggaagaaaaatcaaaaggtaaaagtgaagattcttccaagggtaaaggtaaatcaaagggtgaagatacgactacggatgaagataaacccggaagtggaggtaaacacggaaagggtgagagtaaacacggaaagggtgagagtacacacggaaagggaggtaaacatggaagtgaaggttcgtcaatggatgaaggtaaacacggaggtaagcatggaagcggaggttcgcctatgggtgtaggtaaacacggaagcggaggtaaacatgaaagtggaggttcgcctatgggtggaggtaaacacggaagcggaggtaaacatgaaagtggaggtgcgtctatgggtggaggtaaacacggaagcggaggtagacatgaaggtggaggttcggctatgggtggaggtaagcacggaagtggaggcaaacacggaagtgaaggtaaacacgggggtgagggctcttctatgggtaaaaatagtctatccaaga

SEQ ID NO: 24 - DNA, 706 bp, Sesamum indicum
  source 1..706: mol_type = other DNA
tcacctcaaagtttgctccgttcttgacagaaatggctgagcattatggtcaacaacagcagaccagggcgcctcacctgcagctgcagccgcgcgcccagcgggtagtgaaggcggccaccgccgtgacagccggcggctcgcttctcgtcctctctggcctcactttagccggaactgttattgcgctcaccatcgccactccgctgcttgtgatctttagccccgttctggtgccggcggtcataaccattttcttgctgggtgcgggttttctggcatccggaggcttcggcgtggcggcgctgagtgtgctgtcgtggatttacagatatctgacagggaaacacccgccgggggcggatcagctggaatcggcaaagacgaagctggcgagcaaggcgcgagagatgaaggatagggcagagcagttctcgcagcagcctgttgcggggtctcaaacttcttgagctctttttctgttgtatgaaaatcttaaactgtgtggcccttcgctagttggtttgatgtctggttcagagtgctgattatttggcgacattcggagggcgttggagatgataagctttcaatttgtttttcgaagttaatatttgttcttgattttgatgtctttggtatatatggtcttttagtacattatgttatcaataaacaagtcattttcaaaaaaaaaaaaaaaaaa

SEQ ID NO: 25 - protein, 520 aa, Arabidopsis thaliana
MAILDSAGVTTVTENGGGEFVDLDRLRRRKSRSDSSNGLLLSGSDNNSPSDDVGAPADVRDRIDSVVNDDAQGTANLAGDNNGGGDNNGGGRGGGEGRGNADATFTYRPSVPAHRRARESPLSSDAIFKQSHAGLFNLCVVVLIAVNSRLIIENLMKYGWLIRTDFWFSSRSLRDWPLFMCCISLSIFPLAAFTVEKLVLQKYISEPVVIFLHIIITMTEVLYPVYVTLRCDSAFLSGVTLMLLTCIVWLKLVSYAHTSYDIRSLANAADKANPEVSYYVSLKSLAYFMVAPTLCYQPSYPRSACIRKGWVARQFAKLVIFTGFMGFIIEQYINPIVRNSKHPLKGDLLYAIERVLKLSVPNLYVWLCMFYCFFHLWLNILAELLCFGDREFYKDWWNAKSVGDYWRMWNMPVHKWMVRHIYFPCLRSKIPKTLAIIIAFLVSAVFHELCIAVPCRLFKLWAFLGIMFQVPLVFITNYLQERFGSTVGNMIFWFIFCIFGQPMCVLLYYHDLMNRKGSMS

SEQ ID NO: 26 - protein, 518 aa, Tropaeolum majus
MAVAESSQNTTTMSGHGDSDLNNFRRRKPSSSVIEPSSSGFTSTNGVPATGHVAENRDQDRVGAMENATGSVNLIGNGGGVVIGNEEKQVGETDIRFTYRPSFPAHRRVRESPLSSDAIFKQSHAGLFNLCIVVLIAVNSRLIIENLMKYGWLIDTGFWFSSRSLGDWSIFMCCLTLPIFPLAAFIVEKLVQRNHISELVAVLLHVIVSTAAVLYPVIVILTCDSVYMSGVVLMLFGCIMWLKLVSYAHTSSDIRTLAKSGYKGDAHPNSTIVSCSYDVSLKSLAYFMVAPTLCYQPSYPRSSCIRKGWVVRQFVKLIVFIGLMGFIIEQYINPIVRNSKHPLKGDFLYAIERVLKLSVPNLYVWLCMFYSFFHLWLNILAELLRFGDREFYKDWWNAKTVAEYWKMWNMPVHRWMVRHLYFPCLRNGIPKEGAIIIAFLVSGAFHELCIAVPCHVFKLWAFIGIMFQVPLVLITNYLQEKFSNSMVGNMIFWFIFCILGQPMCVLLYYHDLINLKEK

SEQ ID NO: 27 - protein, 494 aa, Zea mays
MAPPPSMPAASDRAGPGRDAGDSSSLRLRRAPSADAGDLAGDSSGGLRENGEPQSPTNPPPQEQQQHEMLYYRASAPAHRRVKESPLSSDAIFRQSHAGLLNLCIVVLIAVNSRLIIENLMKYGLLIRAGFWFSARSLGDWPLLMCCLTLPVFPLVALMAEKLITRKLIGEHVVILLHIIITTSAIVYPVVVTLKCDSAVLSGFVLMFLASIMWMKLVSYAHTNYDIRVLSKSTEKGAAYGNYVDPENMKDPTFKSLVYFMLAPTLCYQPTYPQTTCIRKGWVTQQLIKCVVFTGLMGFIIEQYINPIVKNSKHPLKGNFLNAIERVLKLSVPTLYVWLCMFYCFFHLWLNIVAELLCFGDREFYKDWWNAKTVEEYWRMWNMPVHKWIIRHIYFPCIRKGFSRGVAILISFLVSAVFHEICIAVPCHIFKFWAFSGIMFQIPLVFLTRYLHATFKHVMVGNMIFWFFFSIVGQPMCVLLYYHDVMNRQAQASR

SEQ ID NO: 28 - protein, 314 aa, Arabidopsis thaliana
MGGSREFRAEEHSNQFHSIIAMAIWLGAIHFNVALVLCSLIFLPPSLSLMVLGLLSLFIFIPIDHRSKYGRKLARYICKHACNYFPVSLYVEDYEAFQPNRAYVFGYEPHSVLPIGVVALCDLTGFMPIPNIKVLASSAIFYTPFLRHIWTWLGLTAASRKNFTSLLDSGYSCVLVPGGVQETFHMQHDAENVFLSRRRGFVRIAMEQGSPLVPVFCFGQARVYKWWKPDCDLYLKLSRAIRFTPICFWGVFGSPLPCRQPMHVVVGKPIEVTKTLKPTDEEIAKFHGQYVEALRDLFERHKSRVGYDLELKIL

SEQ ID NO: 29 - protein, 317 aa, Brassica napus
MGKVRDFGAEDHIPSNIFHAVTAISICLSAIYLNLALVLISLFFLPTSLSLLVLGLLSLFIIIPIDDRSKYGLKLARYICKHAASYFPVTLHVEDYEAFKPDRSYVFGYEPHSVWPIGAVALVDLAGFMPLPNIKLLASNAIFYTPFLRHMWAWLGLASASRKSFSSLLESGYSCILVPGGVQETFHLQHDVENVFLSSRRGFVRIAMEQGAPLVPVFCFGQSRAYKWWKPDCDLYFKLARAIRFTPICFWGVFGSPIPYRHPIHVVVGKPIQVAKSLQPTDEEIDELHGQFVEALKDLFERHKAGAGYSDLQLNIL

SEQ ID NO: 30 - protein, 345 aa, Arachis hypogaea
MEVSGAVLRNVTCPSFSVHVSSRRRGGDSCVTVPVRMRKKAVVRCCCGFSDSGHVQYYGDEKKKENGTAMLSTKKKLKMLKKRVLFDDLQGNLTWDAAMVLMKQLEQVRAEEKELKKKRKQEKKEAKLKASKMNTNPDCESSSSSSSSESESESSESECDNEVVDMKKNIKVGVAVAVADSPRKAETMILYTSLVARDVSANHHHHNAVELFSRNNDISVGSINGGLKNENTAVITTEAIPQKRIEVCMGNKCKKSGSIALLQEFERVVGAEGGAAAAVVGCKCMGKCKSAPNVRIQNSTADKIAEGFNDSVKVPANPLCIGVAWRMLKPLWLRFLGENQESTNE

SEQ ID NO: 31 - protein, 671 aa, Arabidopsis thaliana
MPLIHRKKPTEKPSTPPSEEVVHDEDSQKKPHESSKSHHKKSNGGGKWSCIDSCCWFIGCVCVTWWFLLFLYNAMPASFPQYVTERITGPLPDPPGVKLKKEGLKAKHPVVFIPGIVTGGLELWEGKQCADGLFRKRLWGGTFGEVYKRPLCWVEHMSLDNETGLDPAGIRVRAVSGLVAADYFAPGYFVWAVLIANLAHIGYEEKNMYMAAYDWRLSFQNTEVRDQTLSRMKSNIELMVSTNGGKKAVIVPHSMGVLYFLHFMKWVEAPAPLGGGGGPDWCAKYIKAVMNIGGPFLGVPKAVAGLFSAEAKDVAVARAIAPGFLDTDIFRLQTLQHVMRMTRTWDSTMSMLPKGGDTIWGGLDWSPEKGHTCCGKKQKNNETCGEAGENGVSKKSPVNYGRMISFGKEVAEAAPSEINNIDFRGAVKGQSIPNHTCRDVWTEYHDMGIAGIKAIAEYKVYTAGEAIDLLHYVAPKMMARGAAHFSYGIADDLDDTKYQDPKYWSNPLETKLPNAPEMEIYSLYGVGIPTERAYVYKLNQSPDSCIPFQIFTSAHEEDEDSCLKAGVYNVDGDETVPVLSAGYMCAKAWRGKTRFNPSGIKTYIREYNHSPPANLLEGRGTQSGAHVDIMGNFALIEDIMRVAAGGNGSDIGHDQVHSGIFEWSERIDLKL

SEQ ID NO: 32 - protein, 685 aa, Ricinus communis
MPVIRRKKPTSEPNKNSASDSKTPSEEEEHEQEQEQEEDKNNKKKYPKKKSSEINAKKWSCIDSCCWFVGCICVTWWVLLFLYNAVPASLPQYVTEAITGPLPDPPGVKLKKEGLTAKHPVVFVPGIVTAGLELWEGHQCADGLFRKRLWGGTFGEVYKRPLCWVEHMSLDNETGLDPPGIRVRPVSGLVAADYFAPGYFVWAVLIANLARIGYEEKTMFMASYDWRLSFQNTEVRDQTLSRMKSNIELMVSINGGNKAVIVPHSMGVLYFLHFMKWVEAPAPMGGGGGPDWCAKHIKAVMNIGGPFLGVPKAVAGLFSAEARDIAVARAIAPGFLDNDMFRLQTLQHMMRMSRTWDSTMSMIPRGGDTIWGDLDWSPEEGYIPRKKRQRNNATDNVNEGGAESEISQRKIVRYGRMISFGKNIAEAPSYDIERIDFRDAVKGRSVANNTCLDVWTEYHEMGFGGIKAVAEYKVYTAGSTIELLQFVAPKMMERGSAHFSYGIADNLEDPKYEHYKYWSNPLETKLPNAPEMEIFSMYGVGIPTERAYVYEFSPAAECYIPFQIDTSANDGDEDGCLKDGVYTVDGDETVPVLSAGFMCAKAWRGKTRFNPSGSRTYIREYDHSPPANLLEGRGTQSGAHVDIMGNFALIEDIMRVAAGATGEDLGGDQVYSDIFKWSQKIKLPL

SEQ ID NO: 33 - DNA, 2074 bp, Arabidopsis thaliana
  source 1..2074: mol_type = other DNA
tgaatcctttttcctttcttcttcttcttctcttcagagaaaactttgcttctctttctataaggaaccagacacgaatcccattcccaccgatttcttagcttcttccttcaatccgctctttccctctccattagattctgtttcctctttcaatttcttctgcatgcttctcgattctctctgacgcctcttttctcccgacgctgtttcgtcaaacgcttttcgaaatggcgattttggattctgctggcgttactacggtgacggagaacggtggcggagagttcgtcgatcttgataggcttcgtcgacggaaatcgagatcggattcttctaacggacttcttctctctggttccgataataattctccttcggatgatgttggagctcccgccgacgttagggatcggattgattccgttgttaacgatgacgctcagggaacagccaatttggccggagataataacggtggtggcgataataacggtggtggaagaggcggcggagaaggaagaggaaacgccgatgctacgtttacgtatcgaccgtcggttccagctcatcggagggcgagagagagtccacttagctccgacgcaatcttcaaacagagccatgccggattattcaacctctgtgtagtagttcttattgctgtaaacagtagactcatcatcgaaaatcttatgaagtatggttggttgatcagaacggatttctggtttagttcaagatcgctgcgagattggccgcttttcatgtgttgtatatccctttcgatctttcctttggctgcctttacggttgagaaattggtacttcagaaatacatatcagaacctgttgtcatctttcttcatattattatcaccatgacagaggttttgtatccagtttacgtcaccctaaggtgtgattctgcttttttatcaggtgtcactttgatgctcctcacttgcattgtgtggctaaagttggtttcttatgctcatactagctatgacataagatccctagccaatgcagctgataaggccaatcctgaagtctcctactacgttagcttgaagagcttggcatatttcatggtcgctcccacattgtgttatcagccaagttatccacgttctgcatgtatacggaagggttgggtggctcgtcaatttgcaaaactggtcatattcaccggattcatgggatttataatagaacaatatataaatcctattgtcaggaactcaaagcatcctttgaaaggcgatcttctatatgctattgaaagagtgttgaagctttcagttccaaatttatatgtgtggctctgcatgttctactgcttcttccacctttggttaaacatattggcagagcttctctgcttcggggatcgtgaattctacaaagattggtggaatgcaaaaagtgtgggagattactggagaatgtggaatatgcctgttcataaatggatggttcgacatatatacttcccgtgcttgcgcagcaagataccaaagacactcgccattatcattgctttcctagtctctgcagtctttcatgagctatgcatcgcagttccttgtcgtctcttcaagctatgggcttttcttgggattatgtttcaggtgcctttggtcttcatcacaaactatctacaggaaaggtttggctcaacggtggggaacatgatcttctggttcatcttctgcattttcggacaaccgatgtgtgtgcttctttattaccacgacctgatgaaccgaaaaggatcgatgtcatgaaacaactgttcaaaaaatgactttcttcaaacatctatggcctcgttggatctccgttgatgttgtggtggttctgatgctaaaacgacaaatagtgttataaccattgaagaagaaaagaaaattagagttgttgtatctgcaaaaattttggtagagacacgcgaacccgtttggattttgttatggtgtaaagaaatttcaatcaaaaaactgttgtaataattgttaccaaaaagaaatgcttttctggaaacgaggggaaaaatagtagttttgtt

SEQ ID NO: 34 - DNA, 2090 bp, Tropaeolum majus
  source 1..2090: mol_type = other DNA
acgcggggagttttcaaaatcatattatgctttttcttcactactgcatgaactttctttctacttcttgcaactgatttgtaatccttacacatgtttctagttttctccatataaaaaaaatattctctgagcttctcgattctctagagagagaaggccaaaaaaaaatggcggtggcagagtcgtcacagaacacgacaaccatgagtggtcacggcgactcggatctcaacaatttccgtagaaggaaaccgagttcctccgtgattgaaccttcgtcgtccggttttacatccaccaatggcgtaccggcgactggccacgtggctgagaatcgtgaccaggatcgggtaggggctatggagaacgcaacaggatcggtcaacttaattggaaatggtggaggcgtggttatcgggaatgaagagaaacaggtaggggagactgatatacgattcacttaccggccttcgtttccggctcatcggagggtgagggagagtcctcttagctctgatgcaatcttcaaacagagccatgcgggtttattcaacttgtgtatagtagtgctcattgcagtaaacagtaggcttatcatcgaaaatcttatgaagtatggttggttgatcgatactggtttctggtttagctcaagatcactgggtgattggtccatctttatgtgctgtcttacactcccaattttcccacttgctgcttttattgttgaaaagctggtgcagcgaaatcatatatctgaacttgttgctgttctccttcatgtaatcgtttctaccgctgcagttttatatccagttattgtgatcttaacgtgtgattcggtgtatatgtctggtgtggtattgatgctctttggttgcattatgtggttgaagctggtgtcatatgcacatactagttctgatattagaacactggccaaatctggctataagggggatgcgcaccccaattcaaccattgtgagttgctcatatgatgttagcttgaagagtttggcatacttcatggttgctccgacattatgttaccagcctagctatcctcgttcgtcgtgtatccgcaagggttgggttgttcgtcaatttgtcaaactaatagttttcataggactcatggggttcattatagaacaatatattaatcctatcgttcgaaattccaaacacccattgaaaggagattttttatatgcaatagaaagagttttgaagctttcagttccaaatctatatgtttggctttgcatgttctactcttttttccacctctggttgaacatactggctgagcttcttcgctttggtgatcgtgaattctacaaagattggtggaatgcaaaaactgttgcggagtattggaaaatgtggaatatgcctgttcatagatggatggttcgtcatctatattttccctgtttgaggaatgggatacccaaggaaggtgccattattatcgcgttcttagtttctggtgctttccatgagctctgcattgcagttccttgccacgtattcaagttatgggcctttataggcattatgtttcaggttcccttggtattgattacgaattatctacaagaaaagttcagtaattctatggtgggcaatatgatcttctggttcatcttctgcatacttggccaacctatgtgtgtccttctatattaccatgacctgataaatctaaaggaaaagtgaaaaaatggaagttgcctatgctcagagtattcctatcccaatgcacacattatatggttctgtacaatctgtgcccccttcatcctttacacgtacccatgctggttcctgcacgatgatttgccttttgtttgtaagcaatatttggagagagtccaatttaggaagtgactagtgtggcttatatcttgtatactacctttagtcatgggggggtttttatattactagtaccaaaagtcaagttgtatatgatttacggtttagtttctttcatgttttttgtttttgtgtaaatatacgtttcatatatcactgttttttcaaagtaaaatcaataataccccatagatgttgaaactg

SEQ ID NO: 35 - DNA, 1485 bp, Zea mays
  source 1..1485: mol_type = other DNA
atggccccgcccccctccatgcctgccgcctccgatcgcgccggccctggccgcgacgcgggcgactcgtcctcccttcgcctccgccgcgccccctcagccgacgccggcgaccttgccggcgattcctcgggaggcttgcgggagaacggcgagccgcaatcgccgacgaatccgccgccgcaggagcagcagcagcacgagatgctatactaccgcgcgtcggcgcccgcccaccgccgcgtcaaggagagccccctcagctctgacgccatcttccggcagagccatgctggtcttctgaatctatgcattgttgttctgatcgcagtgaacagcagactcattattgagaatttaatgaagtatggcctgttgataagagctggattttggtttagtgcaagatcgctgggtgactggccccttctaatgtgctgcctcactctaccagttttcccactagttgcactcatggctgagaagctgatcacaagaaagctcattggtgaacatgtggttattctactccatatcattattacaacatctgccattgtctatccagttgttgtgactcttaagtgtgactcagcagtactatctggatttgtgctaatgtttcttgcgagcatcatgtggatgaagcttgtctcttatgcacatacaaattatgatataagggtattgtccaaaagtactgaaaagggtgctgcatatggaaattatgtcgatcctgagaatatgaaagatccaacctttaaaagtctagtgtactttatgttggccccaacactttgttaccagccaacttatcctcaaactacatgtattagaaagggttgggtgacccagcaactcataaagtgcgtggtttttacaggcttgatgggcttcataattgagcaatatataaacccaattgtgaagaattccaaacatccactgaaagggaattttttgaatgctatagaaagagtcttaaaactctcagtgccaacattatatgtatggctttgcatgttctattgcttttttcatttatggctgaacattgtagctgaactcctctgtttcggtgaccgtgaattctataaggactggtggaatgccaaaactgttgaagagtactggaggatgtggaacatgcctgttcataagtggatcatcagacacatatattttccatgtataaggaaaggcttttccaggggtgtagctattctaatctcgtttctggtttcagctgtatttcatgagatatgtattgcggtgccttgccacattttcaaattctgggcattttctgggatcatgtttcagatacccttggtattcttgacaagatatctccatgctacgttcaagcatgtaatggtgggcaacatgatattttggttcttcttcagtatagtcggacagccgatgtgtgtccttctatactaccatgacgtcatgaacaggcaggcccaggcaagtagatag

SEQ ID NO: 36 - DNA, 1330 bp, Arabidopsis thaliana
  source 1..1330: mol_type = other DNA
agttaaaagattggttatttgggctctgcactcaagtgagagagaagatagatagatctgagtagaatcttcgattcattattcgttgtcgtcgttcatctgtgagaagcggacaaaccaaagaatccaccggagctagtgatatgggtggttccagagagttccgagctgaggaacattcaaatcaattccactctatcatcgccatggccatctggcttggcgccattcacttcaacgtcgctcttgttctctgttctctcattttccttcctccttctctatctctcatggtcttgggcttgctctctctgtttatctttatcccaatcgatcatcgtagcaaatatggtcgtaagctcgctaggtacatatgcaagcacgcgtgtaattatttccccgtctctctgtacgtcgaggattacgaagctttccagcctaatcgtgcctatgtctttggttatgaaccacattcggtgctaccgattggagttgttgctctttgtgatctcacagggtttatgcctattcctaacattaaagttcttgcaagtagtgctatattctacactccctttctaaggcatatatggacatggttagggctcaccgctgcttctaggaagaatttcacttcccttttggattctggctacagttgtgttcttgtacctggtggtgtgcaggagacttttcatatgcaacatgatgctgagaatgtcttcctttcaaggagaagaggatttgtgcgcatagccatggaacaggggagccctctggttccagtattctgctttggtcaggcacgcgtgtacaaatggtggaagccggattgtgatctctatcttaaactatctagagcaatcagattcaccccgatctgcttctggggagtttttggatcaccattaccgtgtcgacagcctatgcatgtggtcgttggtaaaccaatagaagtcacaaaaactctgaagccaactgacgaagagattgctaagtttcatggccagtatgtggaagcgcttagggatctgtttgagaggcacaagtcccgagtcggctatgatcttgagctgaaaattctttgaacaaaatctccaatggaaataattacttgtgtgtatccttcattaattgttaccttggagctggatttggacttaatataaatgactacatcatgtagtctacatgtattgcatgtctttagcatcgactgttgaagtaatggaatacgtttataaagcctgtaaattacatgtcgtcttgcacaagagtatgtggtaataataacatttgacccaaaaataatactagttaaatttttcct

SEQ ID NO: 37 - DNA, 954 bp, Brassica napus
  source 1..954: mol_type = other DNA
atgggcaaagtcagagactttggagctgaggatcatatcccatcaaacatattccatgcagtgaccgccatatccatctgcctcagcgccatttacttgaacctcgctcttgttctcatctccctcttcttcctcccaacttctctctccctcctggtcttgggcctgctctctctgtttatcatcatccctatagatgatcgtagcaagtacggtcttaagctggctaggtacatatgcaagcacgcggctagttacttccccgttactctgcatgtcgaagactacgaagctttcaagcctgatcgctcctatgtatttggttatgaaccacactcggtgtggcccattggagctgttgcacttgttgatctggcagggtttatgcctcttcctaacatcaaacttcttgcaagcaatgctatattctacacgccgtttctaaggcacatgtgggcatggttagggctcgcctctgcttctaggaagagtttctcttctcttctggagtctggctatagttgtatccttgtacctggtggtgtgcaggaaacatttcacttgcaacatgatgttgagaacgtcttcctttcatcgagaagaggatttgtgcgcatcgccatggaacaaggggcacctcttgttccagttttctgctttggtcagtcccgtgcgtacaagtggtggaagccggattgtgacctttattttaaactagcaagagcgatcaggtttactcctatctgtttctggggagttttcggatccccaataccatatagacaccctattcatgtggtggttggtaaaccaatacaagttgcaaagtctctgcagccaactgatgaagagattgatgagttgcatggccagtttgtggaagcgcttaaggatctgtttgagaggcacaaggccggagcaggctactctgatctgcagttgaacattctttga

SEQ ID NO: 38 - DNA, 1637 bp, Arachis hypogaea
  source 1..1637: mol_type = other DNA
aatgaacttgacataaagtggttgtttgtaacaccccatttagtgttttgcttagatgttgagagttctataaacttttgtactatttggtaccccgtaattaatagaaatagaaatgtgataatggttctatgtttcattccagaaaaaaattgtcattttaaaaagttttcttaaattctgaatgggaatgatgatcattcagatcaataaggttaacacttttttatatgatattttatgtaatctgattaatttttttttggtgacaaaaaactcgtgccgaattcggcacgaggtcaaaacctcagaagagagaaaaggagaatttggttcctaattaattctcaccatcaacgatggaggtttcaggcgccgttctaaggaatgtcacgtgcccttccttttctgtgcacgtgagttcccgtcgtcgtggtggtgatagttgtgttacagtgccggtgaggatgagaaaaaaggcggtggtgcgttgttgctgcgggttcagtgattcggggcatgtgcagtattacggggacgagaagaagaaggagaatggaaccgctatgttgagcaccaagaagaagctcaagatgctgaagaaacgtgtccttttcgatgatcttcaaggaaacctgacttgggatgctgctatggttttgatgaagcagctagagcaagtaagggcagaggagaaggaattgaagaaaaaaaggaagcaagagaagaaggaggcaaaactcaaagcctctaagatgaacaccaatcctgattgcgaatcgtcatcgtcatcgtcatcatctgaatctgaatctgaatcaagtgagagtgaatgtgacaatgaggtggttgacatgaagaagaacattaaggttggtgttgccgttgctgttgccgattccccacgaaaggcggaaaccatgattctatacacctcccttgttgcccgagatgttagtgctaatcatcatcatcataatgccgtggaattattctctagaaacaatgacatatcagttggaagcattaatggtggccttaagaatgagaatactgcggttattaccactgaagctattcctcagaagaggattgaggtatgcatgggaaacaagtgcaagaaatccggatctattgcattgttgcaagaatttgagagagtggttggtgctgaaggaggtgctgctgctgcagttgttggatgcaagtgcatggggaagtgcaagagtgcacctaatgtgaggattcagaactctactgcagataaaatagctgaggggttcaatgattcagttaaggttccagctaaccctctttgcattggggttgcatggaggatgttgaaaccattgtggcttagattcttgggcgagaatcaggaaagtactaatgaataatttgctggtatgctgtttggaaaattgtatatacgtagtgccagaacctatcagattgttgttttattttatataaacatagactgcatattgttgtgagattcgatttcctcatttattggaacttccagagcctgatttgtgtccattcgagctcgactcaaagatttacatggcctgctcaatctatgaattcaaatttgagggccctgtttggcattaatattaatatattaatat

SEQ ID NO: 39 - DNA, 2811 bp, Arabidopsis thaliana
  source 1..2811: mol_type = other DNA
cccaagtgtaatattgttattaatacatgggctatactaaaagccccacgaaaagtttactgaactatttgaggcccaacaagagcctatcggattaacgcctactgcagaagaaaatctgtctgcactccacccaagaaaacgcagactaattaatgaaatcaacgaaacggataggtcgggtctaaggttgaccatgaaccgcaacctgaaccaggagcaaagtggtcaagttttgccatccggtccgagtcccttggaggaataataccagaacagaaaaaaacagaaaagtcgacaataaacaaaagagacaaatttgatttgattggttccagaaattcgcagagaaacagctctttgtctctctcgactgatctaacaatccctaatctgtgttctaaattcctggacgagatttgacaaagtccgtatagcttaacctggtttaatttcaagtgacagatatgccccttattcatcggaaaaagccgacggagaaaccatcgacgccgccatctgaagaggtggtgcacgatgaggattcgcaaaagaaaccacacgaatcttccaaatcccaccataagaaatcgaacggaggagggaagtggtcgtgcatcgattcttgttgttggttcattgggtgtgtgtgtgtaacctggtggtttcttctcttcctttacaacgcaatgcctgcgagcttccctcagtatgtaacggagcgaatcacgggtcctttgcctgacccgcccggtgttaagctcaaaaaagaaggtcttaaggcgaaacatcctgttgtcttcattcctgggattgtcaccggtgggctcgagctttgggaaggcaaacaatgcgctgatggtttatttagaaaacgtttgtggggtggaacttttggtgaagtctacaaaaggcctctatgttgggtggaacacatgtcacttgacaatgaaactgggttggatccagctggtattagagttcgagctgtatcaggactcgtggctgctgactactttgctcctggctactttgtctgggcagtgctgattgctaaccttgcacatattggatatgaagagaaaaatatgtacatggctgcatatgactggcggctttcgtttcagaacacagaggtacgtgatcagactcttagccgtatgaaaagtaatatagagttgatggtttctaccaacggtggaaaaaaagcagttatagttccgcattccatgggggtcttgtattttctacattttatgaagtgggttgaggcaccagctcctctgggtggcgggggtgggccagattggtgtgcaaagtatattaaggcggtgatgaacattggtggaccatttcttggtgttccaaaagctgttgcagggcttttctctgctgaagcaaaggatgttgcagttgccagagcgattgccccaggattcttagacaccgatatatttagacttcagaccttgcagcatgtaatgagaatgacacgcacatgggactcaacaatgtctatgttaccgaagggaggtgacacgatatggggcgggcttgattggtcaccggagaaaggccacacctgttgtgggaaaaagcaaaagaacaacgaaacttgtggtgaagcaggtgaaaacggagtttccaagaaaagtcctgttaactatggaaggatgatatcttttgggaaagaagtagcagaggctgcgccatctgagattaataatattgattttcgaggtgctgtcaaaggtcagagtatcccaaatcacacctgtcgtgacgtgtggacagagtaccatgacatgggaattgctgggatcaaagctatcgctgagtataaggtctacactgctggtgaagctatagatctactacattatgttgctcctaagatgatggcgcgtggtgccgctcatttctcttatggaattgctgatgatttggatgacaccaagtatcaagatcccaaatactggtcaaatccgttagagacaaaattaccgaatgctcctgagatggaaatctactcattatacggagtggggataccaacggaacgagcatacgtatacaagcttaaccagtctcccgacagttgcatcccctttcagatattcacttctgctcacgaggaggacgaagatagctgtctgaaagcaggagtttacaatgtggatggggatgaaacagtacccgtcctaagtgccgggtacatgtgtgcaaaagcgtggcgtggcaagacaagattcaacccttccggaatcaagacttatataagagaatacaatcactctccgccggctaacctgttggaagggcgcgggacgcagagtggtgcccatgttgatatcatgggaaactttgctttgatcgaagatatcatgagggttgccgccggaggtaacgggtctgatataggacatgaccaggtccactctggcatatttgaatggtcggagcgtattgacctgaagctgtgaatatcatgatctctttaagctgtcctgtcagcttatgtgaatccaatactttgaaagagagatcatcatcaattcatcatcatcgtcatcatcatgatgctcaactcacaaagaagcctgagaatgatactttggtgcgaaattctcaatacctctttaatattcttattgaatgtaaattatacaatcctatctaatgtttgaacgataacacaaaacttgctgcgccatgtttgtttgtcttgtcaaaagcatcaatttgtgggttatacgtagtgtagaggatgattcaaatttgtgataaatttggtaatcaaagttaattctg

SEQ ID NO: 40 - DNA, 2473 bp, Ricinus communis
  source 1..2473: mol_type = other DNA
cttgaattttgtttcgattactttaaaaaaaattgcctttttctgaaagcgcttctagtttgatcttaatggattatttacttttcagaaattagtagctcaaacaaaattaagagagaaaaagagatcgtgggttttttcaaagaaaaagtttcaaacagaaagcacaaactttccgatgtggcttgcgaggaagttggggatgacataaattcttctgccagtccgaagttttggatataaagcagtggtttttgtattttcctttttccttttctgtatcgtttagtaaagtcacatttggcttattgggtttgttttatttcctctgtatttgctttctgtacaaagactatcaataattagttaataagcttaacaaatttaaaaatcttatttttctttaaacccagaaatgcctgtaattcggaggaaaaaacccacttctgaacccaacaaaaattcagcatcagactcaaaaacgccaagcgaggaagaggaacatgaacaagaacaggaacaagaagaagataaaaataacaaaaagaaatacccaaagaagaagagcagtgaaatcaatgcaaaaaaatggtcatgcatagacagctgttgttggtttgttggttgcatctgcgtgacgtggtgggttttactatttctttacaatgcagtgcctgcgtctttgcctcaatacgtaactgaggcaatcacgggtcctttacccgatccacctggtgttaagctgaaaaaagagggattaacagcaaagcatccagtggtttttgtacctgggattgttaccgcggggcttgaattgtgggaaggccatcagtgtgctgatgggctgtttaggaaacggctctggggtggaacttttggagaagtttataagaggcctctctgctgggtagagcatatgtctctagacaatgaaactggattggatcctcctggtataagggtcaggccagtctctggacttgtggctgctgattactttgctccaggctattttgtgtgggctgttctgattgctaatttggcacgcattggatatgaggagaaaacaatgttcatggcctcatacgattggagactttcatttcagaacactgaggtccgtgaccaaacattaagccggatgaagagtaatatagaacttatggtttctatcaatggtggaaataaggcagttattgttccacattccatgggtgttttgtactttctgcattttatgaagtgggttgaggcaccagctccaatgggaggaggtggtggaccagattggtgtgctaagcatatcaaggcagtcatgaacattggtggtccatttttaggtgttcccaaagctgttgctgggcttttctcggctgaagcaagagatattgcagttgccagggccatagcaccaggtttcttagataatgatatgttccgcctacaaacattgcaacacatgatgaggatgtctcgcacatgggattcgaccatgtcaatgataccaagaggtggggacactatctggggcgatcttgattggtcacctgaagaaggttacattcctagaaagaaaaggcagagaaataatgcaactgataatgtaaacgaaggtggggccgaaagtgagatttctcaaagaaagattgttagatatggaagaatgatatcatttgggaaaaatatagcagaggcaccttcatatgatattgaaaggattgactttagggatgctgttaaaggtcgtagtgtggcaaataatacctgccttgatgtgtggactgaataccatgaaatgggattcggaggtattaaagccgttgcagagtataaggtctacactgctggatctactatagagctgcttcagtttgtcgccccaaaaatgatggagcgtggtagtgctcatttttcttatggaattgctgacaatttggaggacccaaaatatgagcactacaaatactggtcaaatcccctggagacaaagttacctaatgctccagaaatggaaatattttccatgtatggagttggcataccaacagaaagagcttatgtttatgagttttctcctgctgctgagtgctacattccatttcagattgatacatcagctaatgatggcgatgaagatggctgtctgaaagatggagtctatactgttgatggggatgagactgttcctgttttaagtgcaggcttcatgtgtgctaaagcttggcgtgggaaaaccagatttaatccttcaggaagtcgaacatacattagagagtacgatcattctcctccagctaatttgctagagggccgaggcacccaaagtggtgcccatgttgatataatgggtaattttgctttaatcgaggatattatgagggtggcagccggggctacaggagaagatttgggaggcgatcaagtgtattcagatatctttaagtggtctcagaagatcaaattaccactgtaa

SEQ ID NO: 41 - DNA, 2753 bp, Oryza sativa
  source 1..2753: mol_type = other DNA
aagcttttggtggtaggaatgtagttttctgacaaagtcaattactgaatataaaaaaaatctgcacagctctgcgtcaacagttgtccaagggatgcctcaaaaatctgtgcagattatcagtcgtcacgcagaagcagaacatcatggtgtgctaggtcagcttcttgcattgggccatgaatccggttggttgttaatctctcctctcttattctcttatattaagatgcataactcttttatgtagtctaaaaaaaaatccagtggatcggatagtagtacgtcatggtgccattaggtaccgttgaacctaacagatatttatgcatgtgtatatatatagctatatagacaaaattggtgccgattatagacccaaaagcaataggtatatataatataatacagaccacaccaccaaactaagaatcgatcaaatagacaaggcatgtctccaaattgtcttaaactatttccgtaggttcagccgttcaggagtcgaatcagcctctgccggcgttttctttgcacgtacgacggacacacatgggcataccatatagctggtccatgacattaggagagagaacgtacgtgttgacctgtagctgagatataacaaggttgattataatatcaccaaacatgaaatcatccaaggatgacccataactatcactactatagtactgcatctggtaaaagaaattgtatagactctatttcgagcactaccacataacgcctgcaatgtgacaccctacctattcactaatgtgcctcttcccacacgctttccacccgtactgctcacagctttaagaaccagaacaaatgagtaatattagtgtcggttcatggctaaaaccagcactgatgtacatgaccacatatgtcaaatgctgcttctaggcatgacccgctcttactaatacctactcatcgctagaagaattttcggctgataaattttcaatttaagcaagagttatccgcgttggttcataactcaaactgatggccccaaccatattagtgcaaatttcacatatgatcataaccttttcatatgaaatcggatcgtgatgaactttatataaacattgtagctgtcgatgatacctacaattttatagttcacaacctttttatttcaagtcatttaaatgcccaaataggtgtttcaaatctcagatagaaatgttcaaaagtaaaaaaggtccctatcataacataattgatatgtaagtgagttggaaaatgataagtacgtgtgagagagatcggagatcaaattctggtgtaataatgtatgtatttcagtcataaaaattggtagcagtagttggggctctgtatatataccggtaaggatgggatggtagtagaataattctttttttgtttttagttttttctggtccaaaatttcaaatttggatcccttacttgtaccaactaatattaatgagtgttgagggcagtagaggtgcaactttaccataatccctctgtttcaggttataagacgttttgactttaaatttgactaagtttatgcgcaaatatagtaatatttataatactaaattagtttcattaaataaataattgaatatattttcataataaatttgtgttgagttcaaaatattattaattttttctacaaacttggtcaaacttaaagcagtttgactttgaccaaagtcaaaacgtcttataacttgaaacggatggattactttttttgtggggacaagtttacaatgtttaataaaagcacaatccatcttaatgttttcaagctgaatattgtaaaattcatggataaaccagcttctaaatgtttaaccgggaaaatgtcgaacgacaaattaatatttttaagtgatggggagtattaattaaggagtgacaactcaactttcaatatcgtactaaactgtgggatttattttctaaaattttataccctgccaattcacgtgttgtagatctttttttttcactaaccgacaccaggtatatcaattttattgaatatagcagcaaaaagaatgtgttgtacttgtaaacaaaaagcaaactgtacataaaaaaaaatgcactcctatataattaagctcataaagatgctttgcttcgtgagggcccaagttttgatgaccttttgcttgatctcgaaattaaaatttaagtactgttaagggagttcacaccaccatcaattttcagcctgaagaaacagttaaacaacgaccccgatgaccagtctactgctctccacatactagctgcattattgatcacaaaacaaaacaaaacgaaataaaaatcagcagcgagagtgtgcagagagagacaaaggtgatctggcgtggatatctccccatccatcctcacccgcgctgcccatcactcgccgccgcatactccatcatgtggagagaggaagacgaggaccacagccagagcccgggtcgagatgccaccacggccacaacccacgagcccggcgcgacaccaccgcgcgcgcgtgagccagccacaaacgcccgcggataggcgcgcgcacgccggccaatcctaccacatccccggcctccgcggctcgcgagcgccgctgccatccgatccgctgagttttggctatttatacgtaccgcgggagcctgtgtgcagagcagtgcatctcaagaagtactcgagcaaagaaggagagagcttggtgagctgcagagtctaga

SEQ ID NO: 42 - DNA, 1664 bp, Oryza sativa
  source 1..1664: mol_type = other DNA
ttatataaaagaaaggatgacttcttatccaaacaaatcctatagtaatgtctttttaactttcagtgactaacatataaaccatcaaacgagtccatattaaaggataatactacgaagaattgtcatcccacatttttacactgccacatatcagttaaaatgaaaaccagctcaccccaagctcaccaagaatcttcgagaaacttataaactccgccgaaaaatctcggacaaacccgcggctcacacgcctcctcgcaccaaaacccacctagaatatcctctccttggccacgcgcgcacatcagctcccaatctcccgccccaggaggcaatcccccctcgcttcccgcgctatttaaactcccgcgccatctccaactcccaactcacactcgctcgctcatcgccatctctctcagctctcacagctcactgcatcaatggccgcggccaccatggcgctctcctccccggtgatggcccgcgcggcgccgtcgacctcctccgcgctcttcggcgaggcgcggatcaccatgcgcaagaccgccgcgaagcccaagccggcggcgtcgtcggggagcccgtggtacggcgccgaccgcgtcctctacctcggcccgctctcgggccgcgagccgccgagctacctcaccggcgagttccccggcgactacgggtgggacaccgcggggctctccgccgacccggagacgttcgccaagaaccgggagctggaggtgatccactcccggtgggcgatgctgggcgcgctcggctgcgtcttcccggagctcctcgcccggaacggcgtcaagttcggcgaggccgtgtggttcaaggcgggctcgcagatcttcagcgagggcgggctcgactacctcggcaacccgagcctgatccacgcgcagagcatcctcgccatctgggcggtgcaggtggtgctcatgggcgccgtcgaggggtaccggattgctggcgggccgctcggcgaggtcgtcgacccgctgtaccccggcggcgccttcgacccgctcggcctcgccgatgaccccgaggcgtgcgcggagctcaaggtgaagaagatcaagaacggccgcctcgccatgttctccatgttcggcttcttcgtccaggccatcgtcaccggcaagggccccctcgagaacctcgccgaccacctcgccgaccccgtcaacaacaacgcctgggcctacgccaccaacttcgtccccggcaagtgaagtggggaccgtagcttagcagtggttaattgtggttggatggatttgtggccagcgagttcgttgtctttgggttggggaagatgggtttagtgcgacgagatgatgatcgagttggtggttgtgtacactaagaagatgaagaagaagatgatgtttttgcaataatgattttattcgtttcccaactaatggtctaggtacttatccgtggtgttattctgattagcggatttctcatctctattagatcggaaacaaatactccctcgatccccaaaatataaccatttctggctatgtatagtataaatatgtagtctaaattgtttatattgtggcatggaaggattaattagtactttaaatttacgtatacaatagattgggtcgaatctaggaagatggtggatccac

SEQ ID NO: 43 - DNA, 1887 bp, Phaseolus vulgaris
  source 1..1887: mol_type = other DNA
gaattcagtgcattgagaaggtgctgaagagggaggaagtatgtgggaggtgcatgattttaaatgggaaggaatgattaaaagagatataaattggctgtcaatgatgaaggatttctatagggttgtgttagtaaatttgaaaattgtattctgttgatattttctgcacaccaatcatgctagatgcccaatggttccttttttatatattctatgattccatactcaccttcaatacatgttttttttttaaaaaaaaggtttgaacaattaactgagttgaatagtagtaaaagcatgaagtgttttgtttttgttttgtggggatgtatgaataagaaaaatcattacaaccagacaaagatgtaactcagaagaaagagtaataagtctatttgcaagaacataaccgtgctaaaatcctttgaaaatacactggaactagctgataattttacagtttccagtggtgcaattctcaaccattaaccaattttatctaaaactatcagctttccagtcatgaacctccataccgaaaatttgtcgcctatttcttttattggcagtggcttttgtcatatttttgccaggctaaattataacagtatcacactctttgaacttcatttaatacctttgaatattgtgttttatagttatctcatggaatgtattttaagataaggttgttgtgaattgtttggttgagtgcatggatttgctgatgaccatagctatttgtggatattatgttttcaaaatgtatgcttggtcattaatcaaccaagaatgataagccccatcatctactaccccttctataccatatacatattttgcttatctacattgcaattcttgctaaacaaatctagcagataacatgcaaactaatgaacttttttagtaatattataaaaatcagccagagtaagctgaagagagaaacacgttttcagctataaaacaaaagggataagagtgtcagttgagtgatatggttctatttttattttttttaattcaaaatcacctttaaaatataaaacattactgtaggaaaaggacaaagggtattcaccataaaagagaaatggtcacaaaatatgtaaggtaaatatggaatgagggcacagtgcaaactactcacaaataacctcaactccaccagcatcacacatttacattcttccaaggaagagataagataatggagctcctccacgtgtcacttccacatggtacctaaggataaggctagctttcaaaattttgctgactcgtgtggccagtatgctgtaatgtcatcacttatagaatccgacggttgtaacatctcagcaagcaatcccctccatctcacaccattggatcagtactatacaaatgatagtattatataaagcaagtaggagcagaagcttgggcatctagcagcagaacagcaagtgattcagaagtaagaagaagaagaaggaaatggcttcttcaatgatctcctcccccgctgtgacgaccgttaaccgtgccggtgccggtgccggtatggtggctcctttcactgggctgaagtccctgggagggttcccaagcaggaagatgaacaatgatattacttccgttgcgaacaacggtggaagagtgcaatgcattcaggtaagagaagatatttatgtgataggaaaatgtgaagatttggaagttggttggttagttagtttgttaactgaattatgatggaaatgaaatgagtaggtgtggccaacagttgggaagaagaagttcgagactctttcgtacctgccacccctgacaaaacaacaattggcaaaggaagtagactaccttcttcggaaaggatgggttccgtgcttggaattc

SEQ ID NO: 44 - DNA, 435 bp, Pisum sativum
  source 1..435: mol_type = other DNA
gatccaaaagcttggacaggaacaaatgttacccatacataaaagatatttgtgaagtaacagtcacaaaattccatgaggccaacatactacaattgaattttcatggatacaattcttacaaaataaaaatatcgacataaccaccatcacacatttacactcttcacatgaaaagataagatcagtgaggtaatatccacatggcactgtcctattggtggcttatgataaggctagcacacaaaatttcaaatcttgtgtggttaatatggctgcaaactttatcattttcactatctaacaagattggtactaggcagtagctaagtaccacaatattaagaccataatattggaaatagataaataaaaacattatatatagcaagttttagcagaagctttgcaattcatacagaagtgagaaaaatg

SEQ ID NO: 45 - DNA, 1666 bp, Pisum sativum
  source 1..1666: mol_type = other DNA
ctgcagtttttcaaagaaattatcttttgttcccttttgccttgtaacttaacaataatggcgtaaataataataaccatgattgacttgaactttggtaaacaattataattaaccatatactagtatattagatttcagtaatcatgaattaaaattgacgagtgcatgtctaaccaaccatcaagtattacttggttcttttcacaatttgtctaccttacctttatcttctttattacacttggttcttttctcaaatttcgacgcgtgttgatgtaatattggtataaacagaaactacaaaacgcagctgtccataacagtcacaatttttctcaaatcttgtggctctcaaacactgtataaaacatgacaaatgtggacccaaaatagagaaagcatagccccataaattaagccattttatgaaaaaataatattatgttgagtcatatatccataagaatccccacagtcacacatggaagagcagcattggatacaaatgatatgaagattttgatccatgaacggattctagaattgcaaagaaaatctccaactagccatagctttagataacacacgataagagcatctgcattataaatacagactcatattcatcttacaaaatcaccattgataaggatacaattatcaaaagcataacaatcttttcaatttcattgcaatataatacacgatggccgcatcatcatcatcatccatggctctctcttctccaaccttggctggcaagcaactcaagctgaacccatcaagccaagaattgggagctgcaaggttcaccatgaggaagtctgctaccaccaagaaagtagcttcctctggaagcccatggtacggaccagaccgtgttaagtacttaggcccattctccggtgagtctccatcctacttgacaggagagttccccggtgactacggttgggacactgccggactctctgctgacccagagacattctccaagaaccgtgagcttgaagtcatccactccagatgggctatgttgggagctttgggatgtgtcttcccagagcttttgtctcgcaacggtgttaaattcggcgaagctgtgtggttcaaggcaggatctcaaatctttagtgagggtggacttgattacttgggcaacccaagcttggtccatgctcaaagcatccttgccatatgggccactcaggttatcttgatgggagctgtcgaaggttaccgtattgccggtgggcctctcggtgaggtggttgatccactttacccaggtggaagctttgatccattgggcttagctgatgatccagaagcattcgcagaattgaaggtgaaggaactcaagaacggtagattagccatgttctcaatgtttggattcttcgttcaagctattgtaactggaaagggtcctttggagaaccttgctgatcatcttgcagacccagtcaacaacaatgcatggtcatatgccaccaactttgttcccggaaaataaacactcttatatttatatgtttttgtgatagtaatcttcttcccaattcaatgtgaattattatcattatcattatcatgtgggtatgcataggttcactaatacaagatcatggatgttttctttttaccaaattttaaattttat

SEQ ID NO: 46 - DNA, 4955 bp, Glycine max
  source 1..4955: mol_type = other DNA
tcgacgtgtacttaaaacctccgccgcagctggtggtgttgaagctgacggtgttgccggtgaactcgaaggagaagatagagattattctgtcatcagtgttggcgggatacaccaaagggttacaccaacacgaggagccaaataaatgcatgtgggttacgccaacagcaccaaggagaaatacccatgcatgcatcattatcaccgatccatagttgtgcatccggtaacgaacttagcggggaagttagttagttgttgcatattaaggaataagaatttagagctgtatgagtcatgagtaatatacagcttagctagctgaacttcgacaatagaaacacagcaacctttccggtcatatttaatttcaatcagaaattttgaaataagggtaatccaattctcatgtaatttatatatttcacaacagttttcaaaattaggaaaaagaaaaataaaaaagtatgaaactaataagttattttatatatttcacaacagttttcagaatttaaaataagagtaatccaattctcatgtatcttttttatttttttttaacactccaaactttaaacacttaaccaacatgatccttgaatatgtaacaagttattattgtctatgatgagttgagttgtttggcaccagttaaaaacttcttaaaattagcctcaaatgaaggattaaaataggtttaatatagattaatttgagcaataaaaataaataaatgaaactttttttagaaagacttgctgacggaaaatgaatagccctcaaaaaattgaaatatgaacaaattgaaaaactaaaatctcaatttaaaaaactatatatatgcatacgggctaacatggttaggggcttaggcttcctcattgtcctaaaagcaaatattcgtttgggccttggttctccagtaagttttttaattgggctcaagaactttaggatttagatgaggatcaataaaaattgatatattgcgcttgcttgttttttttttttcattaaatttacgcaaaaatgaaagaaaaaggtattcacgctcttaaaataaattaataagcagaaattccaaaattttcagttagtccttactaattattaaattatagtattaatccaatgtgattgcggttacatcatgtacggaaaaataattctaatccttgatttaaatttgatcttgactatttatttattctttatttcattttgtaaatcattttatgtatcccctggcaaggcaattttatccaccttgcacccaacacttcgggtcccataaatcaaaccaccttaacttcacacaatgctgtaactcacaccgcccagcatctccaatgtgaaagaagctaaaatttaataaacaatcatacgaagcagtgacaaaataccagatggtattaatgctttgataaaattaattggaaagtataaaatggtaggaaataattaattataattaatttaaataagattaaaaataatttaaaactaaaatgttaaaattttaagaaaattattttaaataatatttaaaaacatttaaaatcattttaaaaaatttatttataggacaattaaatgaatatttcagctaattaaaaacaaaagcttacctagccttagaagacaacttgtccaacaattagatgatacccattgcccttacgttttctttaacatcaattattgtttttgtcaacaagctatcttttagttttattttattggtaaaaaatattgtcgccttcaagttgcatcatttaacacatctcgtcattagaaaaataaaactcttccctaaacgattagtagaaaaaatcattcgataataaataagcaagcaaaattaggaaaaaataacttcattttaaaaaaatcattaaggctatattttttaaatgactaattttatatagactgtaactaaaagtatacaatttattatgctatgtatcttaaagaattacttataaaaatctacggaagaatatcttacaaagtgaaaaacaaatgagaaagaatttagtgggatgattatgattttatttgaaaattgaaaaaataattattaaagactttagtggagtaagaaagctttcctattagtcttttcttatccataaaaaaaaaaaaaaaaatctagcgtgacagcttttccatagattttaataatgtaaaatactggtagcagccgaccgttcaggtaatggacactgtggtcctaacttgcaacgggtgcgggcccaatttaataacgccgtggtaacggataaagccaagcgtgaaggggtgaaggtacatctctgactccgtcaagattacgaaaccgtcaactacgaaggactccccgaaatatcatctgtgtcataaacaccaagtcacaccatacatgggcacgcgtcacaatatgattggagaacggttccaccgcatatgctataaattgcccccacacccctcgaccctaatcgcacttgaattgcaatcaaattagttcattctctttgcgcagttccctacctccccctttcaaggtccgtagattcctcctgtttttttttcttcttctttattgtttgttctacatcagcatgatgttgatttgattgtgttttctatcgtttcatcgattataaattttcataatcagaagattcagcttttattaatgcaagaacgtccttaattgatgattttataaccgtaaattaggtctaattagagtttttttcataaagattttcagatccgtttacaacaagccttaattgttgattctgtagtcgtagattaaggtttttttcatgaactacttcagatccgttaaacaacagccttatttgttgatacttcagtcgtttttcaagaaattgttcagatccgttgataaaagccttattcgttgattctgtatggtatttcaagagatattgctcaggtcctttagcaactaccttatttgttgattctgtggccatagattaggattttttttcacgaaattgcttcttgaaattacgtgatggattttgattctgatttatcttgtgattgttgactctacagatgcagatcttcgtcaagaccctcaccggaaagaccatcacccttgaggtggaaagctctgacaccatcgacaacgtcaaggccaagatccaggacaaggaaggaattcccccggaccagcaacgtctcatctttgccggaaagcagctcgaggacggccgtaccctcgccgactacaacatccagaaggagtcaacccttcaccttgtccttcgtctccgtggtggcatgcagatcttcgtcaagactctcactggcaaaaccataaccctagaggtcgaaagctctgacaccattgacaacgtcaaggccaagatccaggacaaggagggaatccccccagaccagcaacgtctcatcttcgccggaaagcaacttgaggacgggcgcaccctcgccgactacaacatccagaaggaatcaacccttcacctcgtcctccgtctccgtggtggcatgcagatcttcgttaagaccctcaccggcaagactataaccctagaagtcgagagctccgacaccatcgacaacgtcaaggctaagatccaggacaaggagggtatccccccagaccagcagaggctgatcttcgccggcaagcagctcgaggacggacgtacccttgctgactacaacatccagaaggagtcaactctccacttggtgttgcgtcttcgtggtggtatgcagatcttcgtcaagacccttacgggtaagactattaccctcgaggtggagagctccgacaccattgacaatgtgaaggcgaagatccaggacaaggaaggaatcccaccggaccagcagaggcttatttttgctggcaaacagctagaggatggaaggaccctcgctgactacaacatccagaaggagtcaactctgcacttggtgttgcgtcttcgtggggggttttaagctggagctgatttctgtgtgatgttcgatgttggattttcccaaacattttaagaagaaatgtgatgtgtaatgggtctgtaatgacctttgaaaataagtttggtttgtgttgaactctattgtcccattaatgttactactgtttgttatcaatttgtggagctgatatataattatcaactgtatgctggcatattgtgtttgaattttgttctcaatccagtgaattggcgtatattagtgggttttttattcttcatgcttatcacggaggaaaagcagggatgagttgtgtgaaggatggtgatcatcccacgaattattagattttggtattataggttaaagatatttattaggtagggagaactcttggattagattctaaagtgatattttgaaaacacaccaaaggatttttgaacaaacggagatcatgatccctagtttcaattagctgccccttaccaagaggtagcaacttaaaaattatgctcgtgaagcatatttggtatcgtatataaattatgtaagcaatctgcttcagttggaacattaattggcttgcggaatgcacatttagatgatccacacaggtccacataaaaattcctaatcaattttagttatgcaatctttctgcttcaggtagtccttgtcttacagaaagcacattgagatgatccccacacagatcccacataaaaaattcctaatcaataataacttgaaaatcattaaaagatgtaatgccatactaatccctaatttttgtgcaagtttttgtatactgggccccaaaaaataccaaaaagaaaaaacaagttattcagtgatatttttctgctccaatataaatttgattaccaaataccaaaaaataaaaacactattcagtggtatttttctgctccaatactggaccccaaattaattagaatcggatc

SEQ ID NO: 47 - DNA, 1351 bp, Arabidopsis thaliana
  source 1..1351: mol_type = other DNA
aggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagatgcagatctttgttaagactctcaccggaaagacaatcaccctcgag

SEQ ID NO: 48 - DNA, 8024 bp, Cauliflower mosaic virus
  source 1..8024: mol_type = other DNA
ggtatcagagccatgaatcggtttaagaccaaaactcaagagggtaaaacctcaccaaaatacgaaagagttcttaactctaaaaataaaagatctttcaagatcaaacatagttccctcacaccggtgaccgacaggattaccaccgtaaggtttcagaacaacatcgaaagcgtttacgccaacttcgactctcaactcaagtcgtcgtacgatggtagatctaaaaagatcaagactctaagccttaaaaatcttagatgttacgaagccttcctcaggaagtaccttctggaacaataaatctctctgagaatagtactctattgagtatccacaggaaaaataaccttctgtgttgagatggatttgtatccagaagaaaatacccaaagcgagcaatcgcagaattctgaaaataatatgcaaatatttaaatcagaaaattcggatggattctcctccgatctaatgatctcaaacgatcaattaaaaaatatctctaaaacccaattaaccttggagaaagaaaagatatttaaaatgcctaacgttttatctcaagttatgaaaaaagcgtttagcaggaaaaacgagattctctactgcgtctcgacaaaagaattatcagtggacattcacgatgccacaggtaaggtatatcttcccttaatcactaaggaagagataaataaaagactttccagcttaaaacctgaagtcagaaagaccatgtccatggttcatcttggagcggtcaaaatattgcttaaagctcaatttcgaaatgggattgataccccaatcaaaattgctttaatcgatgatagaatcaattctagaagagattgtcttcttggtgcagccaaaggtaatctagcatacggtaagtttatgtttactgtataccctaagtttggaataagccttaacacccaaagacttaaccaaaccctaagccttattcatgattttgaaaataaaaatcttatgaataaaggtgataaagttatgaccataacctatgtcgtaggatatgcattaactaatagtcatcatagcatagattatcaatcaaatgctacaattgaactagaagacgtatttcaagaaattggaaatgtccagcaatctgagttctgtacaatacagaatgatgaatgcaattgggccattgatatagcccaaaacaaagccttattaggagctaaaaccaagactcaaattggtaataaccttcaaataggtaacagtgcttcatcctctaatactgaaaatgaattagctagggtaagccagaacatagatcttttaaagaataaattaaaagaaatctgtggagaataatatgagcattacgggacaaccgcatgtttataaaaaagatactattattagactaaaaccattgtctcttaatagtaataatagaagttatgtttttagttcctcaaaagggaacattcaaaatataattaatcatcttaacaacctcaatgagattgtaggaagaagcttactcggaatatggaagatcaactcatacttcggattaagcaaagacccttcggagtccaaatcaaaaaacccgtcagtttttaatactgcaaaaaccatttttaagagtgggggggttgattactcgagccaactaaaggaaataaaatcccttttagaagctcaaaacactagaataaaaagtctagaaaaagcaattcaatccttagaaaataagattgaaccagagcccttaactaaagaggaagttaaagagctaaaagaatcgattaactcgatcaaagaaggattaaagaatattattggctaaaatggctaatcttaatcagatccaaaaagaagtctctgaaatcctcagtgaccaaaaatccatgaaagcggatataaaagctatcttagaattattaggatcccaaaatcctattaaagaaagcttagaaaccgttgcagcaaaaatcgttaatgacttaaccaagctcatcaatgattgtccttgtaacaaagagatattagaagccttaggtacccaacctaaagagcaactaatagaacaacctaaagaaaaaggtaaaggccttaacttaggaaaatactcttaccccaattacggagtaggaaatgaagaattaggatcctctggaaaccctaaagctttaacctggcccttcaaagctccagcaggatggccgaatcaattttagacagaaccattaataggttttggtataatctgggagaagattgtctctcagaaagtcaattcgatcttatgataagattgatggaagagtcccttgacggggaccaaattattgatctaacctctctacctagtgataatttgcaggttgaacaggttatgacaactaccgaagactcaatctcggaagaagaatcagaattccttctagcaataggagaaacatctgaagaagaaagcgattcaggagaagaacctgaattcgagcaagttcgaatggatcgaacaggaggaacggagattccaaaagaagaagatggtgaaggaccatctagatacaatgagagaaagagaaagaccccggaggaccggtactttccaactcaaccaaagaccattccaggacaaaagcaaacgtctatgggaatgctcaacattgactgccaaaccaatcgaagaactctaatcgacgactgggcagcagaaatcggattgatagtcaagaccaatagagaagactatctcgatccagaaacaattctactcttgatggaacacaaaacatcaggaatagccaaggagttaatccgaaatacaagatggaaccgcactaccggagacatcatagaacaggtgatcgatgcgatgtacaccatgttcttaggactaaactactccgacaacaaagttgctgagaagattgacgagcaagagaaggccaagatcagaatgaccaagctccagctctgcgacatctgctaccttgaggaatttacatgtgattatgaaaagaacatgtataagacagaactggcggatttcccaggatatatcaaccagtacctgtcaaaaatccccatcattggagaaaaagcgttaacacgctttaggcatgaagctaacggaaccagcatctacagtttaggtttcgcggcaaagatagtcaaagaagaactatctaaaatctgcgacttatccaagaagcagaagaagttgaagaaattcaacaagaagtgttgtagcatcggagaagcttcaacagaatatggatgcaagaagacatccacaaagaagtatcacaagaagcgatacaagaaaaaatataaggcttacaaaccttataagaagaaaaagaagttccgatcaggaaaatacttcaagcccaaagaaaagaagggctcaaagcaaaagtattgcccaaaaggcaagaaagattgcagatgttggatctgcaacattgaaggccattacgccaacgaatgtcctaatcgacaaagctcggagaaggctcacatccttcaacaagcagaaaaattgggtctccagcccattgaagaaccctatgaaggagttcaagaagtattcattctagaatacaaagaagaggaagaagaaacctctacagaagaaagtgatggatcatctacttctgaagactcagactcagactgagcaggtgatgaacgtcaccaatcccaattcgatctacatcaagggaagactctacttcaagggatacaagaagatagaacttcactgtttcgtagacacgggagcaagcctatgcatagcatccaagttcgtcataccagaagaacattgggtcaatgcagaaagaccaattatggtcaaaatagcagatggaagctcaatcaccatcagcaaagtctgcaaagacatagacttgatcatagccggcgagatattcagaattcccaccgtctatcagcaagaaagtggcatcgatttcattatcggcaacaacttctgtcagctgtatgaaccattcatacagtttacggatagagttatcttcacaaagaacaagtcttatcctgttcatattgcgaagctaaccagagcagtgcgagtaggcaccgaaggatttcttgaatcaatgaagaaacgttcaaaaactcaacaaccagagccagtgaacatttctacaaacaagatagaaaatccactagaagaaattgctattctttcagaggggaggaggttatcagaagaaaaactctttatcactcaacaaagaatgcaaaaaatcgaagaactacttgagaaagtatgttcagaaaatccattagatcctaacaagactaagcaatggatgaaagcttctatcaagctcagcgacccaagcaaagctatcaaggttaaacccatgaagtatagcccaatggatcgcgaagaatttgacaagcaaatcaaagaattactggacctaaaagtcatcaagcccagtaaaagccctcacatggcaccagccttcttggtcaacaatgaagccgagaagcgaagaggaaagaaacgtatggtagtcaactacaaagctatgaacaaagctactgtaggagatgcctacaatcttcccaacaaagacgagttacttacactcattcgaggaaagaagatcttctcttccttcgactgtaagtcaggattctggcaagttctgctagatcaagaatcaagacctctaacggcattcacatgtccacaaggtcactacgaatggaatgtggtccctttcggcttaaagcaagctccatccatattccaaagacacatggacgaagcatttcgtgtgttcagaaagttctgttgcgtttatgtcgacgacattctcgtattcagtaacaacgaagaagatcatctacttcacgtagcaatgatcttacaaaagtgtaatcaacatggaattatcctttccaagaagaaagcacaactcttcaagaagaagataaacttccttggtctagaaatagatgaaggaacacataagcctcaaggacatatcttggaacacatcaacaagttccccgatacccttgaagacaagaagcaacttcagagattcttaggcatactaacatatgcctcggattacatcccgaagctagctcaaatcagaaagcctctgcaagccaagcttaaagaaaacgttccatggagatggacaaaagaggataccctctacatgcaaaaggtgaagaaaaatctgcaaggatttcctccactacatcatcccttaccagaggagaagctgatcatcgagaccgatgcatcagacgactactggggaggtatgttaaaagctatcaaaattaacgaaggtactaatactgagttaatttgcagatacgcatctggaagctttaaagctgcagaaaagaattaccacagcaatgacaaagagacattggcggtaataaatactataaagaaatttagtatttatctaactcctgttcattttctgattaggacagataatactcatttcaagagtttcgttaatctcaattacaaaggagattcgaaacttggaagaaacatcagatggcaagcatggcttagccactattcatttgatgttgaacacattaaaggaaccgacaaccactttgcggacttcctttcaagagaattcaataaggttaattcctaattgaaatccgaagataagattcccacacacttgtggctgatatcaaaaggctactgcctatttaaacacatctctggagactgagaaaatcagacctccaagcatggagaacatagaaaaactcctcatgcaagagaaaatactaatgctagagctcgatctagtaagagcaaaaataagcttagcaagagctaacggctcttcgcaacaaggagacctctctctccaccgtgaaacaccggaaaaagaagaagcagttcattctgcactggctacttttacgccatctcaagtaaaagctattccagagcaaacggctcctggtaaagaatcaacaaatccgttgatggctaatatcttgccaaaagatatgaattcagttcagactgaaattaggcccgtaaagccatcggacttcttacgtccacatcagggaattccaatcccaccaaaacctgaacctagcagttcagttgctcctctcagagacgaatcgggtattcaacaccctcataccaactactacgtcgtgtataacggacctcatgccggtatatacgatgactggggttgtacaaaggcagcaacaaacggtgttcccggagttgcgcataagaagtttgccactattacagaggcaagagcagcagctgacgcgtatacaacaagtcagcaaacagataggttgaacttcatccccaaaggagaagctcaactcaagcccaagagctttgcgaaggccttaacaagcccaccaaagcaaaaagcccactggctcatgctaggaactaaaaagcccagcagtgatccagccccaaaagagatctcctttgccccagagatcacaatggacgacttcctctatctctacgatctagtcaggaagttcgacggagaaggtgacgataccatgttcaccactgataatgagaagattagccttttcaatttcagaaagaatgctaacccacagatggttagagaggcttacgcagcaggtctcatcaagacgatctacccgagcaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtggtgaatataaaccagacacgagacgactaaacctggagcccagacgccgtttgaagctagaagtaccgcttaggcaggaggccgttagggaaaagatgctaaggcagggttggttacgttgactcccccgtaggtttggtttaaatatcatgaagtggacggaaggaaggaggaagacaaggaaggataaggttgcaggccctgtgcaaggtaagacgatggaaatttgatagaggtacgttactatacttatactatacgctaagggaatgcttgtatttaccctatataccctaatgaccccttatcgatttaaagaaataatccgcataagcccccgcttaaaaaatt

SEQ ID NO: 49 - protein, 145 aa, Sesamum indicum
MACHYGQQQQTCAPHLQLQPRACRVVKAATAVTAGGSLLVLSGLTLAGTVIALTIATPLLVIFSPVLVPAVITIFLLGAGFLASGGFGVAALSVLSWIYRYLTGKHPPGADCLESAKTKLASCAREMKDRAEQFSCQPVAGSQTS

SEQ ID NO: 50 - protein, 518 aa, Tropaeolum majus
MAVAESSQNTTTMSGHGDSDLNNFRRRKPSSSVIEPSSSGFTSTNGVPATGHVAENRDQDRVGAMENATGSVNLIGNGGGVVIGNEEKQVGETDIRFTYRPSFPAHRRVRESPLSSDAIFKQSHAGLFNLCIVVLIAVNSRLIIENLMKYGWLIDTGFWFSSRSLGDWSIFMCCLTLPIFPLAAFIVEKLVQRNHIAELVAVLLHVIVSTAAVLYPVIVILTCDSVYMSGVVLMLFGCIMWLKLVSYAHTSSDIRTLAKSGYKGDAHPNSTIVSCSYDVSLKSLAYFMVAPTLCYQPSYPRSSCIRKGWVVRQFVKLIVFIGLMGFIIEQYINPIVRNSKHPLKGDFLYAIERVLKLSVPNLYVWLCMFYSFFHLWLNILAELLRFGDREFYKDWWNAKTVAEYWKMWNMPVHRWMVRHLYFPCLRNGIPKEGAIIIAFLVSGAFHELCIAVPCHVFKLWAFIGIMFQVPLVLITNYLQEKFSNSMVGNMIFWFIFCILGQPMCVLLYYHDLINLKEK

SEQ ID NO: 51 - protein, 147 aa, Glycine max
MAELHYQQQHQYPHRYPNDPYEQQTSYSTQVVKAATAVTAGGSLLILASLILAGTIIALTIVTPPLVIFSPVLVPAVITVALLSLGFLASGGFGVAAITVLAWIYRYVTGKYPPGADQLDSAPHKIMDKAREIKDYGQQQISGVQAS

SEQ ID NO: 52 - DNA, 799 bp, Glycine max
  source 1..799: mol_type = other DNA
ctaccacattaattactcactcttcactcaaaactcttgtctttcattttgataattcccacactatggctgagcttcactaccaacaacaacaccaataccctcaccgataccctaatgatccatacgaacaacaaactagctactccactcaggtcgtcaaggcggccaccgccgtcaccgcgggcggctccctcttgatcctcgcctcgttgatccttgccggcaccatcatcgccctcaccatcgtcacaccaccgctcgtcatcttcagtccggttctcgtccccgcggtgatcaccgtcgcgctgctgagcctggggttccttgcctccggcgggttcggtgtggcggcgatcacggtgctggcgtggatctacaggtacgtcaccgggaagtacccacctggcgcggatcagttggacagcgcgcctcacaagatcatggacaaggcgcgtgagatcaaggactatggacagcagcaaatcagtggggtgcaggcttcttaatcgtgtgcacgcacgcacccgatcaaatccatctcgtggatttctctctttgtctagttagttatatttacgatttatgaatgaatgaatgcaggaatgagcgttaaagttgaatttgaatctcctcttttctttgtattttcgtttattgtgggtgttcgatcatcatcacttgttgtagttgtaatgtcttaattttgtgtgcatgctgcgactattcaaggacgatccatttgtgtatttattctagttttttctttccatttttatgaatcggtttgcttgttttc

SEQ ID NO: 53 - protein, 226 aa, Glycine max
MTTQVPPHSVQVHTTTHRYEAGVVPPGARFEPPRYEAGVKAPSIYHSERGPTTSQVLAVLAGLPVGGILLLLAGLTLAGTLTGLAVATPLFVLFSPVLVPATVAIGLAVAGFLTSGAFGLTALSSFSWILNYIRETQPASENLAAAAKHHLAEAAEYVGQKTKEVGQKTKEVGQDIQSKAQDTREAAARDAREAAARDAREAAARDAKVEARDVKRTTVTATTATA

SEQ ID NO: 54 - DNA, 1058 bp, Glycine max
  source 1..1058: mol_type = other DNA
aacacacaccccactaacaattccttcacttgcagcactgttgcatcatcatcttcattgcaaaaccctaaacttcaccttcaaccatgaccacacaagtaccaccacacagtgtccaagtccacacaacaacacaccgctacgaagctggcgtcgtcccccccggagcccgtttcgaaccgccgcgttatgaagccggcgtcaaagcgccctccatttaccattccgagagaggtccaacgacttcccaggtcctcgccgtcctcgccggcctcccggtcggtggcatcctgctcctcctggcgggattgaccctggcaggaaccctcaccggactggcagttgccacgccgctcttcgtcctcttcagcccggtgctggttccagccacggtcgccatcggcctggccgtggccgggttcctgacgtcgggggccttcgggctcacggcgctgtcttccttctcctggatcctgaactacatccgggagacccagccagcctcggagaacctcgccgccgccgcgaagcatcacctcgccgaggcggcggagtacgtggggcagaagacgaaggaagtggggcagaagaccaaggaggttgggcaggatattcagagcaaggcacaggatacaagggaggcagctgcaagggatgcaagggaggcagctgcaagggatgcaagggaagctgctgcaagggatgcaaaggtggaggcaagggatgtaaagagaacaacagtgacagcaacaaccgcaaccgcatgatgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacttccattgttgaattcaatgtcaaatgtgttttgcgttaaaaaaaaaaaaaaaaaa

SEQ ID NO: 55 - protein, 165 aa, Glycine max
MATISDQPRGSYSYGTSYGAPYGTTYETNTSINNPPSRQTVKFITAATIGITLLLLSGLTLTGTVIGLIIATPLLVIFSPILVPAAFVLFLVASGFLFSGGCGVAAIAALSWIYNYVSGNQPAGYDTLDYAKGYLADKARDVKERAKDYGSYAQGRINEATQGAY

SEQ ID NO: 56 - DNA, 857 bp, Glycine max
  source 1..857: mol_type = other DNA
acactcgttactctcatcacattattctatatcaattttggttaagagtcttgaaaaagcaagagaaaaaaaaataccaaccatggcaaccatttctgatcaacctagagggtcctactcctatggaacctcatatggagcaccctatggaaccacgtatgagaccaacaccagcattaacaacccaccttcacgccaaacagtgaagttcataactgctgccactattggcatcacactcttgctcctgtctgggttgaccctcacaggcactgtcataggtttgatcattgcaacccctcttcttgttatcttcagccccatccttgtccctgctgcgtttgtgctgttcctagttgcttctggctttttgttctctgggggctgtggtgtggctgccattgctgctttgtcttggatttacaactatgtgtctgggaaccagcctgcgggttatgacacccttgactatgcaaaagggtaccttgccgataaggcaagggatgtaaaggagagggcaaaggattatggaagttatgctcaaggtagaattaatgaggccacacaaggagcttattagtcatgcctgcttatatatgcatgagtgtatgaggttgaattgaaggtggtgatggtggtggtacccttgtggatttgcatgttcaagttctgcagtttattttgaatcttgtttgcattgttctatgtagttctggtttttgtaatctcgtttgtttttgaagctaaatcgttcgccttatttgtgtaaccatcatgttaataaatctcttttgtagctagatcaatgattccttccctcttgttgttaaaatgtgatttgggttgtttcctcgttt

SEQ ID NO: 57 - protein, 147 aa, Glycine max
MAELHYQPQHQYPLRYPNDPHQQTRSSTHQVVKAATAVTAGGSLLILASLILAATVIALTIVTPLFVIFSPVLVPAVITVALLSLGFLASSGFGVAAITVLAWIYRYVTGKQPPGADQLDSARHKIMDKAREIKDYGQQQISGVQAS

SEQ ID NO: 58 - DNA, 802 bp, Glycine max
  source 1..802: mol_type = other DNA
atcacactaatcactcactcttcactcaaaactcttctctttcattttcataattcccacacattatggctgagcttcactaccaaccacaacaccaataccccctccgctaccctaacgatccacaccaacaaacccgttcctccacccaccaggtcgtcaaggccgccaccgccgtcacagcgggcggctccctcttgatcctcgcctcgttgatcctcgccgccaccgtcattgccctcaccatcgtcaccccgctgttcgtcattttcagtccggttctcgtccccgccgtaatcaccgttgcgctcctgagcctggggttcctcgcctcgagtgggttcggtgtggcggcgatcacggtgctggcgtggatctacaggtacgtcaccggcaagcaaccacctggcgcggatcagttagacagcgcgcgtcacaagatcatggacaaggcgcgtgagatcaaggactacggacagcaacaaatcagcggggtccaggcttcttaattctgcactcgcacgcacatgatcgtatccatctcgtggaatatttctctctttgtctagttagtttatatttaccatttatgaatgaatgaatgcatgattgagcgttaaagttgaatttgaatctctcttttctttgtattttcgtttattgtgggtgttcgctcatcatcacttgttgtaatgtcttaattttgtgtgcatgctgcgactattcaaggacgatccatttgtgtttttattctactcttatttatttatttatttcctttttttatgaaaaaaaaaaaaaa

SEQ ID NO: 59 - protein, 156 aa, Zea mays
MADHHRGATGGGGGYGDLQRGGGMHGEAQQQQKQGAMMTALKAATAATFGGSMLVLSGLILAGTVIALTVATPVLVIFSPVLVPAAIALALMAAGFVTSGGLGVAALSVFSWMYKYLTGKHPPGADQLDHAKARLASKARDIKDAAQHRIDQAQGS

SEQ ID NO: 60 - DNA, 882 bp, Zea mays
  source 1..882: mol_type = other DNA
atctatcgcactcgtagtcgtagctcaagcatcagcggcaggagctctgggcagcgtgcgcacgtggggtacctagctcgctctgctagcctaccatggctgatcaccaccggggtgcgacgggaggtggcgggggctacggcgacctccagcgcgggggcggcatgcacggcgaggcgcagcagcagcagaagcagggcgccatgatgacggcgctcaaggccgcgacggccgcgaccttcggcgggtcgatgctggtgctgtccgggctgatcctggccggcaccgtgatcgcgctcacggtggccacccccgtgctggtgatcttcagcccggtgctggtgccggccgccatcgcgctggcgctcatggcggccgggttcgtcacctccggcggcctcggcgtcgctgcgctgtccgtgttctcctggatgtacaagtacctgacgggcaagcacccgccgggcgccgaccagctggaccacgccaaggcgaggctggcgtccaaggcccgcgacatcaaggatgcagcacagcaccgcatcgaccaggcgcaggggtcttgagagaagaaccacactcgagcggaccgcgcgcgcgacctccttgaaccactgccggcgcggcggcatatggcccttaaaggcggtggctgctgctacgtacgctgccgtagagtctcggtcgccgcgatagctctagctagtcgtttatgtgttgtgctttgtgtgtgcatgcttgtgtctgggggcatgcagtgcagtgcagtactatatgctgtatgcgtctctctttgatcggagaggcggatgtacagcatgctcgatatgtctagtttggatgtcatgtttatgatgaggaataaaatgcagtgttcaggtg

SEQ ID NO: 61 - protein, 504 aa, Glycine max
MAISDEPESVATALNHSSLRRRPSATSTAGLFNSPETTTDSSGDDLAKDSGSDDSINSDDAAVNSQQQNEKQDTDFSVLKFAYRPSVPAHRKVKESPLSSDTIFRQSHAGLFNLCIVVLVAVNSRLIIENLMKYGWLIKSGFWFSSKSLRDWPLFMCCLSLVVFPFAAFIVEKLAQRKCIPEPVVVVLHIIITSTSLFYPVLVILRCDSAFVSGVTLMLFSCVVWLKLVSYAHTNYDMRALTKLVEKGEALLDTLNMDYPYNVSFKSLAYFLVAPTLCYQPSYPRTPYIRKGWLFRQLVKLIIFTGVMGFIIEQYINPIVQNSQHPLKGNLLYATERVLKLSVPNLYVWLCMFYCFFHLWLNILAELLRFGDREFYKDWWNAKTVEDYWRMWNMPVHKWMIRHLYFPCLRHGLPKAAALLIAFLVSALFHELCIAVPCHIFKLWAFGGIMFQVPLVLITNYLQNKFRNSMVGNMIFWFIFSILGQPMCVLLYYHDLMNRKGKLD

SEQ ID NO: 62 - DNA, 1978 bp, Glycine max
  source 1..1978: mol_type = other DNA
agaaatctgttttttttttttttttggagaagagaagactgagttagaaaacacgctcggtcttcttctccaatggcgatttccgatgagcctgaaagtgtagccactgctctcaaccactcttccctgcgccgccgtccctccgccacctccaccgccggcctcttcaattcgcctgagacaaccaccgacagttccggtgatgacttggccaaggattctggttccgacgactccatcaacagcgacgacgccgccgtcaattcccaacagcaaaacgaaaaacaagacactgatttctccgtcctcaaattcgcctaccgtccttccgtccccgctcaccgcaaagtgaaggaaagtccgctcagctccgacactattttccgtcagagtcacgcgggcctcttcaacctttgtatagtagtccttgttgctgtgaatagccgactcatcattgagaatttaatgaagtatggttggttgatcaaatctggcttttggtttagttcaaagtcattgagagactggccccttttcatgtgttgtctttctcttgtggtatttcctttcgctgcctttatagtggagaagttggcacaacggaagtgtatacccgaaccagttgttgttgtacttcatataatcattacctcaacttcgcttttctatccagttttagttattctcaggtgtgattctgcttttgtatcaggtgtcacgttaatgctgttttcttgtgttgtatggttaaaattggtgtcttatgcacatacaaactatgatatgagagcacttaccaaattagttgaaaagggagaagcactgctcgatactctgaacatggactatccttacaacgtaagcttcaagagcttggcatatttcctggttgcccctacattatgttaccagccaagctatcctcgcacaccttatattcgaaagggttggttgtttcgccaacttgtcaagctgataatatttacaggagttatgggatttataatagaacaatatattaatcccatagtacaaaattcacagcatcctctcaagggaaaccttctttacgccaccgagagagttctgaagctttctgttccaaatttatatgtgtggctctgcatgttctattgctttttccacctttggttaaatatcctggcagagcttcttcgatttggtgatcgtgaattctacaaggattggtggaatgccaaaactgtcgaagattattggaggatgtggaatatgcctgttcacaaatggatgatccgccacctatattttccatgtttaaggcacggtctaccaaaggctgctgctcttttaattgccttcctggtttctgctttattccatgagctgtgcattgctgttccttgccacatattcaagttgtgggctttcggtggaattatgtttcaggttcctttggtcttgatcactaattatctgcaaaataaattcagaaactcaatggttggaaatatgattttttggttcatattcagtatccttggtcaacctatgtgtgtactgctatactaccatgacttgatgaataggaaaggcaaacttgactgaagctacggccattacattttaaaggtgcacatggatgagcttttcagttttcagattgtaaaattgatgtggatatgttggtcaatatttgttttctacgaatgctttcatctaccatggcattggctgctctgaaggaattccacgggatatgccagttcacgaggctaattcattatcttgatctatgtacttaccaactctcctctggcaattgtatcaaaatatgcaattttgagagccatacactggcattgataactgccaaggaacactctaactgttttctgttaactgttaattagtagagggctagatgtaaatggtttatgctcaatatatttatttcctcctagtcttcaagttccaaaaaaaaaaaaaaaaaaaaaaa